(19) United States
(12) Patent Application Publication
(10) Pub. No.: US 2024/0309451 A1
(43) Pub. Date: Sep. 19, 2024

(54) DIAGNOSTICS AND THERAPEUTICS FOR EBV IN MS AND OTHER AUTOIMMUNE DISEASES

(71) Applicants: THE BOARD OF TRUSTEES OF THE LELAND STANFORD JUNIOR UNIVERSITY, Stanford, CA (US); THE UNITED STATES GOVERNMENT AS REPRESENTED BY THE DEPARTMENT OF VETERANS AFFAIRS, Washington, DC (US)

(72) Inventors: William H. Robinson, Palo Alto, CA (US); Tobias V. Lanz, Redwood City, CA (US)

(21) Appl. No.: 18/267,038

(22) PCT Filed: Dec. 23, 2021
(86) PCT No.: PCT/US2021/065064

§ 371 Date: Jun. 13, 2023

Related U.S. Application Data
(60) Provisional application No. 63/131,581, filed on Dec. 29, 2020.

(51) Int. Cl. C12Q 1/6883; C07K 16/08; G01N 33/569

(52) U.S. Cl. CPC C12Q 1/6883; C07K 16/085; G01N 33/56972; C07K 2317/31; C07K 2317/33; C07K 2317/34; C07K 2317/565

(57) ABSTRACT

Compositions and methods are provided for diagnosis and treatment of individuals having multiple sclerosis (MS) or MS spectrum disorders. It is shown herein that EBV-transformed B cells, and particularly plasmablasts, are present in human MS spinal fluid. These cells produce antibodies. e.g. IgG antibodies, that selectively bind to EBV EBNA-1 sequences, including without limitation residues 386-405, and cross-react with the myelin protein hepacam/glialcam, including without limitation residues 337-385.

### CROSS REFERENCE TO RELATED APPLICATION

The present application claims the benefit of and priority to U.S. Provisional Patent Application No. 63/131,581, filed Dec. 29, 2020, the entire disclosure of which is hereby.

### STATEMENT OF GOVERNMENT SUPPORT

This invention was made with Government support under contract NIH:AR063676 awarded by the National Institution of Health. The Government has certain rights in the invention.

### INTRODUCTION

In Multiple Sclerosis (MS), autoreactive B and T cells cause tissue-specific destruction of myelin in the central nervous system (CNS). The presence of oligoclonal bands (OCB) in cerebro-spinal fluid (CSF) and the efficacy of B cell depleting therapies emphasized the importance of B cells in MS pathology, which act by autoantibody secretion and T cell activation during antigen presentation. Anti-CD20 B cell depleting therapeutics have emerged as efficacious therapeutics available for both relapsing remitting and primary progressive MS. Nevertheless, many aspects of B cell immunology in MS are not well understood, including their phenotypic and functional characteristics in CNS, CSF, and blood, and the degree to which autoantibody production vs. B-cell dependent T cell activation contribute to pathogenicity. Both mechanisms are B cell receptor (BCR)-dependent. However, the identification of dominant B cell antigens has been notoriously difficult in MS.

Viral proteins have been suggested to contribute to and even initiate inflammation by eliciting molecular mimicry to myelin proteins. Epstein-Barr virus (EBV) in particular has been associated with MS, as 99.5% of MS patients (vs. 93.6%-94.4% of healthy individuals) have been infected with EBV-often years prior to disease onset. Infectious mononucleosis and MS share the same major risk allele (HLA-DRB1*15:01), and infectious mononucleosis as well as serum immunoglobulin reactivity against EBV nuclear antigen 1 (EBNA1) are independent and synergistic risk factors for MS. Immunodominant MS-associated epitopes of EBNA1 have been identified, and candidates for molecular mimicry have been proposed, including anoctamin-2, but research has not advanced beyond the descriptive identification of EBV/CNS cross-reactivities in CSF and sera of MS patients, and fallen short on in-depth characterization of pathogenic epitopes and assessment of their functional relevance in-vivo. Further, the role of EBV in promoting the activation of pathogenic B cells remains poorly understood.

### SUMMARY

Compositions and methods are provided for diagnosis and treatment of individuals having multiple sclerosis (MS) or MS spectrum disorders. It is shown herein that EBV-transformed B cells, and particularly plasmablasts, are present in human MS spinal fluid. These cells produce antibodies, e.g. IgG antibodies, that selectively bind to EBV EBNA-1 sequences, including without limitation residues 386-405 of EBNA-1, and cross-react with the myelin protein hepacam/glialcam, including without limitation residues 337-385 of hepacam/glialcam. Phosphorylation of glialcam at one or both of residues S376 and S377 can enhance binding affinity. The cross-reaction with glialcam induces neuroinflammation and can exacerbate symptoms of multiple sclerosis. Elevated anti-GlialCAM serum reactivity is shown to be present in MS patients in comparison to healthy individuals.

In some embodiments, an individual is diagnosed for the presence of EBV-driven MS pathology, where the diagnosis may be combined with treatment according to the diagnosis. Without being limited by theory, it is believed that reactivation of EBV in B cells, including plasmablasts, drives pathogenic activity. Detection of markers associated with active EBV infection, e.g. in MS patients, is indicative of EBV-driven MS pathology. In some embodiments active EBV infection is detected in peripheral B cell populations. In some embodiments active EBV infection is detected in CSF B cell populations. Methods for detection of active EBV infection can include, without limitation, detection of EBV proteins on the surface of B cells, where such markers include, without limitation: BILF-1, LMP1 and LMP2. Methods for detection of active EBV infection can include determining the presence of transcripts associated with active infection. Latent infection is characterized by limited expression of viral proteins, apart from, for example EBNA1, LMP1 and LMP2. Active infection can result in expression of a broader range of viral proteins, including for example BILF-1, LMP1, LMP2, etc. Detection of such proteins or transcripts can be indicative of an EBV-driven MS pathology.

In other embodiments, an individual is diagnosed for the presence of EBV-driven MS pathology by detecting the presence of antibodies in one or both of serum and CSF with specificity for glialcam. In some embodiments, the specificity is for an epitope cross-reactive with EBNA-1. In some embodiments the antibodies detected are IgG antibodies. The determination is optionally combined with detection of active EBV infection. A variety of methods may be utilized for the detection of antibodies.

An individual diagnosed for EBV-driven MS pathology is optionally treated in accordance with the finding. Treatment to reduce the adverse symptoms can include, without limitation, targeting all B cells for depletion; targeting EBV-infected B cells for depletion; inhibiting EBV-transformed B cells; inhibiting the B cell activating functions of certain EBV-encoded proteins, including but not limited to LMP1 and LMP2; and tolerizing the individual for glialcam epitopes, e.g. cross-reactive glialcam epitopes.

Knowledge of cross-reactive autoantigens, e.g. glialcam, can be used to develop specific therapies and diagnostics for MS, in place of the non-specific immunomodulation that is conventionally used. The present invention provides an important candidate antigen for being involved in pathogenesis of MS; and provides a target for diagnosis and therapeutic intervention. Cross-reactive peptides also find use in tolerization strategies, e.g. to decrease pathogenic responses through altered peptide ligands (APLs), manipulation of dendritic cell responses, biasing T cell responses to non-pathogenic responses, and the like.

Depletion of pathogenic B cells may comprise, for example, targeting antibodies to markers present on actively infected B cells, e.g. target pathogenic EBV-infected B cells, e.g. by therapies directed to one or more of cell-surface EBV proteins: BILF-1, LMP1 and LMP2. Antibodies may be conjugated to a cytotoxic agent, e.g. tubulin polymerization inhibitors, e.g. maytansinoids (maytansine), dolastatins, auristatin drug analogs, cryptophycin; duocarmycin derivatives, e.g. CC-1065 analogs, duocarmycin; enediyne antibiotics, e.g. esperamicin, calicheamicin; pyrrolobenzodiazepine (PBD); and the like. In other embodiments, other targeted agents are utilized include anti-B cell antibodies, e.g. anti-CD20 antibodies, anti-CD38 antibodies e.g. rituximab; anti-CD19 antibodies; EBV-specific CAR T cells, and the like. In a favored embodiment, anti-EBV LMP1, LMP2 and BILF1 monoclonal antibodies, alone or in combination, are used to deplete EBV-infected pathogenic B cells.

Inhibition of B cells with active EBV infection may utilize, for example, inhibition of specific tyrosine kinase proteins. Such inhibitor include, without limitation, BTK inhibitors. BTK signaling influences antigen presentation on B cells and is essential to the production of antibodies, proinflammatory cytokines and chemokines, and cell adhesion molecules. Examples of useful inhibitors include ibrutinib, evobrutinib, PRN2246 (SAR442168), BIIB091, and other BTK inhibitors.

### BRIEF DESCRIPTION OF THE DRAWINGS

The invention is best understood from the following detailed description when read in conjunction with the accompanying drawings. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIGS. 1A-K: Phenotype and repertoire differences of B cells in blood and CSF. (A-F): Flow cytometry data. (A) Number of plasmablasts as % of all B cells in blood and CSF. (B) VLA-4 expression and (C) HLA-DR expression in non-plasmablast (red) and plasmablast (blue) in blood vs. CSF. (D) Representative flow cytometry data (patient MS37) comparing HLA-DR expression on non-plasmablast (red) and plasmablast (blue) in blood (upper panel) and CSF (lower panel). (E,F) Immunoglobulin classes in (E) plasmablast and (F) non-plasmablast B cells in blood (red) vs. CSF (blue). (A-F) p values according to unpaired Student's t tests. (G-I) Single-cell BCR repertoire sequencing data, (G) clonality, percent of clonal sequences are shown in blood (red, larger numbers of sequences down-sampled to match CSF sequences) vs. CSF (blue), p values according to Mann-Whitney U test. (H) Overview of individual PB BCR repertoires, showing clonality, size of individual clonal expansions, and immunoglobulin classes in blood (upper panel) vs. CSF (lower panel), numbers indicate number of sequences, inner circle: colored wedges represent clonal expansions and grey area represents singleton antibody sequences, outer circle: immunoglobulin classes, red: IgG, blue: IgA, green: IgM, sequence locations in outer circle correspond to inner circle. (I) IGHV gene distribution in blood vs. CSF PB, p according to Student's T tests, Holm-Sidak adjusted p-values: *<0.05, **<0.01, ***<0.0001. (J,K) Mass spectrometry data of purified immunoglobulins from CSF samples in singleton BCR B cells (red) vs. clonally expanded B cells (blue), (J) percent of VDJ sequences that could be uniquely identified with mass spectrometry in the respective groups (PSM cutoff: 1), (K) percent of VDJ sequences that were highly abundant in CSF (≥10 PSM). (J,K) p values according to Mann-Whitney U test.

FIGS. 2A-L: CSF mAB reactivity to EBV proteins, EBNA1 epitope mapping, and structure of EBNA1-antibody complex. (A) Heatmap showing mAB reactivities (z-scores) to viral lysates and EBV proteins and (B) to EBNA1 peptides. Selected mABs are shown with highest reactivities to respective antigens. IE: immediate early, E: early, and L: late lytic stage. (C) Western blot of recombinant EBNA1 (full-length and truncated proteins), showing coomassie staining (top panel) and staining with MS39p2w174 (bottom panel). (D) MS39p2w174 tested on an ELISA-based alanine-scan of EBNA1 with 90 peptides (20mers, 13AA overlap). (E) EBNA1 AA386-405, logo representation showing the contribution of each residue to binding of MS39p2w174, as assessed by alanine-scan. (F-J) Crystal structure of MS39p2w174 in complex with EBNA1 AA386-405. (F) Cartoon and stick representation, showing EBNA1 AA393-401 in the binding groove. Additional peptide residues are truncated for better visualization. HC: red/brown colors, LC: blue/cyan, CDR loop colors correspond to annotations in G. (G) View of the binding groove from the top. Surface representation of the Fab with EBNA1 AA386-402 in stick representation. (H-J) Cartoon and stick representation outlining close interactions. Major H-bond forming residues are represented as sticks. H-bonds <3.1 Å are represented as black dashed lines. (I) Magnification of peptide in hydrophobic cage, (J) magnification of region around Arg396 to emphasize polar contacts of residues in the HC with Arg396 and Arg397. (K,L) (E) Bio-layer interferometry measurement of MS39p2w174 (blue) and germline (GL, red) affinity to EBNA1 full-length protein. (K) KD in nM, (L) association and dissociation curves. P according to unpaired Student's t test.

FIGS. 3A-P: Molecular Mimicry between EBNA1 and GlialCAM. (A) Heatmap showing top 16 results of Huprot array for MS39p2w174, compared to 3 control mABs, sorted from top to bottom by the ratio of MS39p2w174/average of controls (left column, min: 89, max: 911). Raw counts are shown in right four columns (min: 1, max: 36450). (B) ELISA measuring binding of MS39p2w174 and two control mABs to recombinant proteins EBNA1 AA328-641 as well as GlialCAM AA34-416 (full length) and A262-416 (intracellular domain, ICD). (C) Western blot GlialCAM full-length vs. ICD. Top panel: MS39p2w174, bottom panel: commercial anti-GlialCAM antibody (anti-extracellular domain). (D, E) Bio-layer interferometry measurement of MS39p2w174 affinity to GlialCAM ICD (AA262-416), (D) KD in nM, (E) association and dissociation curves, p according to unpaired Student's t test. (E) Logo plot, showing alignment of amino acid sequences of EBNA1 AA386-405 and GlialCAM AA370-389 with central epitope. (I) ELISA data showing binding of MS39p2w174 to EBNA1 AA386-405 and GlialCAM AA370-389 non-phosphorylated and phosphorylated at the indicated serine residues. (G,H) Prediction of disorder with PONDR for (G) EBNA1 and (H) GlialCAM. High scores indicate disorder, red bar: epitope region. (J,K) Bio-layer interferometry measurement of MS39p2w174 affinity to GlialCAM 20mer peptides. (J) KD in μM, (K) association and dissociation curves, pSer: phosphorylated Serine residues, p according to unpaired Student's t test. (L) Heatmap showing mAB reactivities (MFI) to GlialCAM proteins, peptides, and phosphorylated peptides as well as cross-reactivities to EBNA1 and other EBV proteins, ICD: intracellular domain, ECD: extracellular domain, pSer: phosphorylated serine residues. (M-O) ELISA data showing human plasma reactivities against (M) EBNA1 protein, (N) EBNA1 AA386-405, and (O) GlialCAM protein, p according to unpaired Student's t test. (P) Plasma reactivity to EBNA1 AA386-405, blocked with indicated proteins or peptides, p according to unpaired Student's t test.

FIGS. 4A-C: Relevance of EBNA1/GlialCAM cross-reactivity in a mouse model of MS. (A) Experimental autoimmune encephalomyelitis (EAE) scores of mice immunized with EBNA1 AA386-405 (red) and scrambled peptide control (blue), * p<0.05 (Mann-Whitney U test). (B) Mouse serum ELISA showing IgG reactivities against EBNA1 AA386-405 (top panel) and GlialCAM AA370-389 (bottom panel) in groups immunized with EBNA1 AA386-405 (red), scrambled peptide (blue), and PBS (black).

FIGS. 5A-F. Details of B cell phenotypes in blood and CSF. (A, B) Flow cytometry gating strategy for B cells, representative plots from (A) blood and (B) CSF (patient MS30). (C) B cell subsets as % of all B cells in blood (red) and CSF (blue). p according to unpaired Student's t test. (D) representative histogram showing integrin a4 expression in non-PB B cells (red) and PB (blue) in blood (top panel) and CSF (lower panel) (patient MS37). (E,F) HLA-DR expression on non-PB B cells (red) and PB (blue) in (E) blood and (F) CSF, in patients carrying HLA-DR15 vs. other HLA-genotypes (non-HLA-DR15), ns=non-significant, according to unpaired Student's t test.

FIGS. 6A-I. Extended BCR repertoire data. (A-F) Single-cell BCR repertoire sequencing data, (A) overview of individual BCR repertoires, comparison of CSF PB to non-PB B cells with respect to clonality, size of individual clonal expansions, and immunoglobulin classes in all CSF B cells (upper panel), CSF plasmablasts (middle panel), and CSF non-plasmablast B cells (lower panel), numbers indicate number of sequences, inner circle: colored wedges represent clonal expansions and grey area represents singleton antibody sequences, outer circle: immunoglobulin classes, red: IgG, blue: IgA, green: IgM, sequence locations in outer circle correspond to inner circle. <5 non-PB cells were sequenced from individual MS12 and only PB were sorted from MS39, therefore the respective samples were excluded from this figure. (B) IGHV and IGLV cumulated mutation count in PB in blood (red) vs. CSF (blue). (C) Mean HC CDR3 lengths (amino acid sequences) of PB in blood (red). vs. CSF (blue), (B,C) means±standard deviations across patients as well as means of individual patients are shown, p values according to unpaired Student's t test. (D-F) Immunoglobulin gene distributions in blood vs. CSF plasmablasts for (D) IGLV, (E) IGHJ, and (F) IGLJ, p according to Student's t tests, Holm-Sidak adjusted p-values: ***<0.0001. (G,H) Mass spectrometry data of purified immunoglobulins from CSF samples, comparing non-plasmablast B cells (red) with plasmablasts (blue), (G) percent of VDJ sequences that could be uniquely identified with mass spectrometry in the respective group (PSM cutoff: 1), (H) percent of VDJ sequences that were highly abundant in CSF (≥10 PSM). (G,H) p values according to Mann-Whitney U test. (I) Single-cell sequencing efficacy in non-plasmablast B cells (red) vs. PB (blue) in CSF. Fraction of sequences that passed filter thresholds are shown as percentages of the number of sorted cells in the respective group.

FIG. 7: Representative phylogenetic tree of patient MS37: CSF sequences (top half-circle) and blood sequences (bottom half-circle) are depicted. Each leaf represents the full-length HC and LC sequence of a B cell. Sequences are sorted from the interior to the exterior first by IGHV families, then by IGHV genes, and then concatenated HC/LC sequences are clustered. IGHV families, clonality, immunoglobulin class, plasmablast vs. non-plasmablast, mutation counts, and expressed sequences are indicated according to figure legend.

FIGS. 8A-B: CSF mAB reactivity to EBV peptides. (A) Heatmap showing mAB reactivities (z-scores) to EBV virus lysates and recombinant EBV proteins as well as other virus lysates. (B) Heatmap showing mAB reactivities (MFI) to GlialCAM proteins, peptides, and phosphorylated or citrullinated peptides. Results for all tested mABs and proteins/peptides are shown. IE: immediate early, E: early, and L: late lytic/activated stage, pSer: phosphorylated serine residue, Cit: citrulline residue, _B-_E: duplicate probes of same/similar lysates and proteins tested in different preparations or batches.

FIG. 9: CSF mAB reactivity to EBV peptides: Heatmap showing mAB reactivities (z-scores) to EBV peptides. Results for all tested mABs and peptides are shown. ICD: intracellular domain, ECD: extracellular domain, PM: peptide mix.

FIGS. 10A-G. mAB reactivity to EBV peptides and structural data for EBNA1 AA386-405/MS39p2w174 complex. (A) Heatmap showing mAB reactivities (z-scores) of selected mABs (as in FIG. 2A) against the selected reactive peptide antigens. ICD: intracellular domain, ECD: extracellular domain, PM: peptide mix. (B) ELISA-based alanine-scan on EBNA1 AA386-405, corresponding to FIG. 2E. Mean±standard deviation is shown from triplicate repeats of one representative out of 3 independent experiments. (C) 20× image of protein crystals in hanging drop. (D) Asymmetric unit containing two peptide-Fab complexes in a diagonal orientation, red/pink: HC, blue/cyan: LC, black/gray: peptide. (E) EBNA1 peptide and its 2mFo-DFc map (contoured at 10) are shown, depicted on HC (cyan) and LC (pink) in surface representation. (F,G) Amino acid sequences of variable regions of mAB MS39p2w174 (F) HC and (G) LC. Bold font: CDR, regular font: framework regions (FR), GL: germline with variable genes indicated, only germline residues that differ from MS39p2w174 sequence are shown, red: residues that closely interact with EBNA1 AA386-405, according to crystal structure, dots: gaps introduced during IMGT GapAlign for alignment and numbering purposes, numbers: residue numbers according to IMGT unique numbering.

FIGS. 11A-C. GlialCAM expression in human tissues: (A) Expression levels of GlialCAM in human organs (source: proteinatlas.org). (B) phosphorylation of single residues (source: phosphosite.org), (C) ELISA data: reactivities of MS39p2w174 to citrullinated versions of GlialCAM AA370-389.

FIGS. 12A-G. T cell activation and phenotype in response to immunization with EBNA1 AA389-405: (A) T cell proliferation and (B-G) ELISA measurement of indicated cytokines in response to indicated stimuli, in group immunized with scrambled peptide (blue) and EBNA1 AA386-405 (red), p according to unpaired Student's t test.

FIG. 13. MHC types of MS patients in which B cell repertoire sequencing was performed.

FIGS. 14-B. LMP-1. (A) Schematic depiction of LMP-1, showing 6 transmembrane domains and 3 extracellular domains. (B) Protein sequence, intracellular, transmembrane, and extracellular domains are annotated.

FIGS. 15A-B. LMP-2. (A) Schematic depiction of LMP-2, showing 12 transmembrane domains and 6 extracellular domains. (B) Protein sequence, intracellular, transmembrane, and extracellular domains are annotated.

FIG. 16A-B. BILF-1. (A) Schematic depiction of BILF-1, showing 7 transmembrane domains and 4 extracellular domains. (B) Protein sequence, intracellular, transmembrane, and extracellular domains are annotated.

FIG. 17. Overview of expression and function of LMP-1, LMP-2, and BILF-1. All three proteins are encoded by EBV genes and expressed as membrane proteins with accessible extracellular domains. LMP-1 and LMP-2 activate B cells by mimicking endogenous B cell activating signaling pathways. BILF-1 inhibits MHC class I expression and thereby inhibits anti-EBV T cell responses. Individual or combinations of monoclonal antibodies specific for LMP1; or LMP2; or BILF1; or LMP1+BILF1; or LMP2+BILF1; or LMP1+LMP2; or LMP1+LMP2+BILF1; can be used to deplete EBV-infected B cell to treat EBV infectious syndromes, EBV-mediated cancers, and/or EBV-driven autoimmune diseases.

### DETAILED DESCRIPTION OF THE EMBODIMENTS

The instant disclosure provides methods for the stratification of MS-patients for EBV-driven pathology. It is shown that hepacam/glialcam is cross-reactive with EBNA-1 epitopes and can drive such pathology. Tolerizing vaccines and methods of using such vaccines are provided for treating individuals having multiple sclerosis, systemic lupus erythematosus, Sjogren's Sydrome, type I diabetes, rheumatoid arthritis and other autoimmune diseases associated with EBV-infection. Aspects of the methods include administering to the individual, in need thereof, agents to deplete or inhibit pathogenic B cells. Aspects also include administration of an effective amount of an hepacam/glialcam tolerizing vaccine to reduce one or more symptoms of MS. Compositions and kits for practicing the methods of the disclosure are also provided.

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present inventions have been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

Compositions and methods are provided that relate to the characterization, use, and manipulation of immunogenic peptides associated with autoimmune disease; and pathogenic B cells reactive with such immunogenic peptides.

The subject methods may be used for diagnostic, prophylactic or therapeutic purposes. As used herein, the term “treating” is used to refer to both prevention of relapses, and treatment of pre-existing conditions. For example, the prevention of autoimmune disease may be accomplished by administration of the agent prior to development of a relapse. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) reducing the symptoms of the disease, i.e., causing regression of the disease or symptom. The treatment of ongoing disease, where the treatment stabilizes or improves the clinical symptoms of the patient, is of particular interest.

“Inhibiting” the onset of a disorder shall mean either lessening the likelihood of the disorder's onset, or preventing the onset of the disorder entirely. Reducing the severity of a relapse shall mean that the clinical indicia associated with a relapse are less severe in the presence of the therapy than in an untreated disease. As used herein, onset may refer to a relapse in a patient that has ongoing relapsing remitting disease. The methods of the invention can be specifically applied to patients that have been diagnosed with autoimmune disease, including for example autoimmune disease. Treatment may be aimed at the treatment or reducing severity of relapses, which are an exacerbation of a pre-existing condition.

“Diagnosis” as used herein generally includes determination of a subject's susceptibility to a disease or disorder, determination as to whether a subject is presently affected by a disease or disorder, prognosis of a subject affected by a disease or disorder (e.g., identification of disease states, stages of disease, or responsiveness of disease to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood, cerebral spinal fluid, and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, cerebrospinal fluid (CSF), biological fluids, and tissue samples.

The terms “individual,” “subject,” “host,” and “patient,” used interchangeably herein and refer to any mammalian subject for whom diagnosis, treatment, or therapy is desired, for example humans, non-human primate, mouse, rat, guinea pig, rabbit, etc. Mammals other than humans can be advantageously used as subjects that represent animal models of inflammation. A subject can be male or female.

The term “agent” as used herein includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). In particular the cell surface markers of the present teachings can be analyzed by any of various conventional methods known in the art. To “analyze” can include performing a statistical analysis to, e.g., determine whether a subject is a responder or a non-responder to a therapy (e.g., administration of a peptide or antibody treatment as described herein).

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal, intramuscular, subcutaneous, and the like.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Immune tolerance, or immunological tolerance, or immunotolerance, is a state of unresponsiveness of the immune system to substances or tissue that have the capacity to elicit an immune response in a given organism. A tolerogenic regimen or formulation is a regimen or formulation that induces tolerance to an antigen of interest, e.g. tolerance to autoantigens such as myelin basic protein. A tolerogenic dose is the dose of an agent, e.g. peptide, altered peptide ligand, DNA vector, etc. that is sufficient to decrease undesirable immune responsiveness to a target antigen. A tolerogenic DNA construct is a DNA construct that encodes a tolerogenic peptide(s) that decreases undesirable immune responsiveness to a target antigen. A tolerogenic peptide is a peptide that acts to decrease undesirable immune responsiveness to a target antigen.

Tolerance can be induced through an immunization protocol developed to activate suppressive immune responses against an antigen. Tolerance is classified into central tolerance or peripheral tolerance depending on where the state is originally induced—in the thymus and bone marrow (central) or in other tissues and lymph nodes (peripheral).

Immune tolerance encompasses the range of physiological mechanisms by which the body reduces or eliminates an immune response to particular agents. It is used to describe the phenomenon underlying discrimination of self from non-self, suppressing allergic responses, allowing chronic infection instead of rejection and elimination, and preventing attack of fetuses by the maternal immune system.

Peripheral tolerance develops after T and B cells mature and enter the peripheral tissues and lymph nodes. It is established by a number of partly overlapping mechanisms that mostly involve control at the level of T cells, especially CD4+ helper T cells, which orchestrate immune responses Reactivity toward certain antigens may be reduced by induction of tolerance after repeated exposure, or exposure in a certain context. In these cases, there can be a differentiation of naïve CD4+ helper T cells into induced Treg cells (iTreg cells) in the peripheral tissue or nearby lymphoid tissue (lymph nodes, mucosal-associated lymphoid tissue, etc.).

### EBV

The Epstein-Barr virus (EBV), formally called Human gammaherpesvirus 4, is one of the nine known human herpesvirus types in the herpes family, and is one of the most common viruses in humans. It is best known as the cause of infectious mononucleosis (“mono” or “glandular fever”).

Infection with EBV occurs by the oral transfer of saliva and genital secretions. Most people become infected with EBV and gain adaptive immunity. In the United States, about half of all five-year-old children and about 90% of adults have evidence of previous infection. Infants become susceptible to EBV as soon as maternal antibody protection disappears. Many children become infected with EBV, and these infections usually cause no symptoms or are indistinguishable from the other mild, brief illnesses of childhood. In the United States and other developed countries, many people are not infected with EBV in their childhood years. When infection with EBV occurs during adolescence, it causes infectious mononucleosis in approximately 35 to 50% of infected individuals.

EBV infects B cells of the immune system and epithelial cells. Once EBV's initial lytic infection is brought under control, EBV latency persists in the individual's B cells for the rest of their life. When EBV infects B cells in vitro, lymphoblastoid cell lines eventually emerge that are capable of indefinite growth. The growth transformation of these cell lines is the consequence of viral protein expression. EBNA-2, EBNA-3C, and LMP-1 are essential for transformation, whereas EBNA-LP and the EBERs are not. Following natural infection with EBV, the virus is thought to execute some or all of its repertoire of gene expression programs to establish a persistent infection. Given the initial absence of host immunity, the lytic cycle produces large numbers of virions to infect other (presumably) B-lymphocytes within the host.

The latent programs reprogram and subvert infected B-lymphocytes to proliferate and bring infected cells to the sites at which the virus presumably persists. Eventually, when host immunity develops, the virus persists by turning off most (or possibly all) of its genes, only occasionally reactivating to produce fresh virions. A balance is eventually struck between occasional viral reactivation and host immune surveillance removing cells that activate viral gene expression.

### Disease Conditions

This invention relates to autoimmune diseases, cancers, and infectious conditions associated with EBV infection, and in which EBV drives pathogenic B cell responses.

### Autoimmune Diseases

Autoimmune diseases associated with EBV infection include multiple sclerosis (MS), systemic lupus erythematosus (SLE), type I diabetes (T1D), Sjogren's Syndrome, rheumatoid arthritis (RA), dermatomyositis (DM), and other autoimmune diseases.

Multiple sclerosis (MS) is characterized by various symptoms and signs of CNS dysfunction, with remissions and recurring exacerbations. Classifications of interest for analysis by the methods of the invention include relapsing remitting MS (RRMS), primary progressive MS (PPMS) and secondary progressive MS (SPMS). The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat can accentuate symptoms and signs.

Systemic lupus erythematosus (SLE). Lupus, technically known as systemic lupus erythematosus (SLE), is an autoimmune disease in which the body's immune system mistakenly attacks healthy tissue in many parts of the body. Symptoms vary between people and may be mild to severe. Common symptoms include painful and swollen joints, fever, chest pain, hair loss, mouth ulcers, swollen lymph nodes, feeling tired, and a red rash which is most commonly on the face. Often there are periods of illness, called flares, and periods of remission during which there are few symptoms.

The cause of SLE is not clear. It is thought to involve genetics together with environmental factors. Among identical twins, if one is affected there is a 24% chance the other one will be as well. Female sex hormones, sunlight, smoking, vitamin D deficiency, and certain infections are also believed to increase the risk. The mechanism involves an immune response by autoantibodies against a person's own tissues. These are most commonly anti-nuclear antibodies and they result in inflammation. Diagnosis can be difficult and is based on a combination of symptoms and laboratory tests. There are a number of other kinds of lupus erythematosus including discoid lupus erythematosus, neonatal lupus, and subacute cutaneous lupus erythematosus.

Sjogren's Syndrome (SjS, SS). Sjögren's syndrome is a long-term autoimmune disease that affects the body's moisture-producing (lacrimal and salivary) glands, and often seriously affects other organs systems, such as the lungs, kidneys, and nervous system. Primary symptoms are dryness (dry mouth and dry eyes), pain and fatigue Other symptoms can include dry skin, vaginal dryness, a chronic cough, numbness in the arms and legs, feeling tired, muscle and joint pains, and thyroid problems. Those affected are also at an increased risk (5%) of lymphoma.

Type I diabetes (T1D). Type 1 diabetes, previously known as juvenile diabetes, is a form of diabetes in which very little or no insulin is produced by the islets of Langerhans in the pancreas. Insulin is a hormone required for the body to use blood sugar. Before treatment this results in high blood sugar levels in the body. The classic symptoms are frequent urination, increased thirst, increased hunger, and weight loss. Additional symptoms may include blurry vision, tiredness, and poor wound healing. Symptoms typically develop over a short period of time, often a matter of weeks.

The cause of type 1 diabetes is unknown, but it is believed to involve a combination of genetic and environmental factors. Risk factors include having a family member with the condition. The underlying mechanism involves an autoimmune destruction of the insulin producing beta cells in the pancreas. Diabetes is diagnosed by testing the level of sugar or glycated hemoglobin (HbA1C) in the blood. Type 1 diabetes can be distinguished from type 2 by testing for the presence of autoantibodies.

There is no known way to prevent type 1 diabetes. Treatment with insulin is required for survival. Insulin therapy is usually given by injection just under the skin but can also be delivered by an insulin pump. A diabetic diet and exercise are important parts of management. If left untreated, diabetes can cause many complications. Complications of relatively rapid onset include diabetic ketoacidosis and nonketotic hyperosmolar coma. Long-term complications include heart disease, stroke, kidney failure, foot ulcers and damage to the eyes. Furthermore, complications may arise from low blood sugar caused by excessive dosing of insulin.

Rheumatoid arthritis (RA). Rheumatoid arthritis is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. This may result in a low red blood cell count, inflammation around the lungs, and inflammation around the heart. Fever and low energy may also be present. Often, symptoms come on gradually over weeks to months.

While the cause of rheumatoid arthritis is not clear, it is believed to involve a combination of genetic and environmental factors. The underlying mechanism involves the body's immune system attacking the joints. This results in inflammation and thickening of the joint capsule. It also affects the underlying bone and cartilage. The diagnosis is made mostly on the basis of a person's signs and symptoms. X-rays and laboratory testing may support a diagnosis or exclude other diseases with similar symptoms. Other diseases that may present similarly include systemic lupus erythematosus, psoriatic arthritis, and fibromyalgia among others.

The goals of treatment are to reduce pain, decrease inflammation, and improve a person's overall functioning. This may be helped by balancing rest and exercise, the use of splints and braces, or the use of assistive devices. Pain medications, steroids, and NSAIDs are frequently used to help with symptoms. Disease-modifying antirheumatic drugs (DMARDs), such as hydroxychloroquine and methotrexate, may be used to try to slow the progression of disease. Biological DMARDs may be used when disease does not respond to other treatments. However, they may have a greater rate of adverse effects. Surgery to repair, replace, or fuse joints may help in certain situations.

Dermatomyositis (DM). Dermatomyositis is a long-term inflammatory disorder which affects skin and the muscles. Its symptoms are generally a skin rash and worsening muscle weakness over time. These may occur suddenly or develop over months. Other symptoms may include weight loss, fever, lung inflammation, or light sensitivity. Complications may include calcium deposits in muscles or skin.

The cause is unknown. Theories include that it is an autoimmune disease or a result of a viral infection. It is a type of inflammatory myopathy. Diagnosis is typically based on some combination of symptoms, blood tests, electromyography, and muscle biopsies.

While no cure for the condition is known, treatments generally improve symptoms. Treatments may include medication, physical therapy, exercise, heat therapy, orthotics and assistive devices, and rest. Medications in the corticosteroids family are typically used with other agents such as methotrexate or azathioprine recommended if steroids are not working well. Intravenous immunoglobulin may also improve outcomes. Most people improve with treatment and in some the condition resolves completely.

### At-Risk Individuals for Autoimmune Disease

In some embodiments the methods of the invention comprise treating, isolating cell populations from, or diagnosing individuals “at-risk” for development of, or in the “early-stages” of, an autoimmune disease. “At risk” for development of an autoimmune disease includes: (1) individuals whom are at increased risk for development of an autoimmune disease, and (2) individuals exhibiting a “pre-clinical” disease state, but do not meet the diagnostic criteria for the autoimmune disease (and thus are not formally considered to have the autoimmune disease).

Individuals “at increased risk” for development (also termed “at-risk” for development) of an autoimmune disease are individuals with a higher likelihood of developing an autoimmune disease or disease associated with inflammation compared to the general population. Such individuals can be identified based on their exhibiting or possessing one or more of the following: a family history of autoimmune disease; the presence of certain genetic variants (genes) or combinations of genetic variants which predispose the individual to such an autoimmune disease; the presence of physical findings, laboratory test results, imaging findings, marker test results (also termed “biomarker” test results) associated with development of the autoimmune disease, or marker test results associated with development of a metabolic disease; the presence of clinical signs related to the autoimmune disease; the presence of certain symptoms related to the autoimmune disease (although the individual is frequently asymptomatic); the presence of markers (also termed “biomarkers”) of inflammation; and other findings that indicate an individual has an increased likelihood over the course of their lifetime to develop an autoimmune disease or disease associated with inflammation. Most individuals at increased risk for development of an autoimmune disease or disease associated with inflammation are asymptomatic, and are not experiencing any symptoms related to the disease that they are at an increased risk for developing.

Included, without limitation, in the group of individuals at increased risk of developing an autoimmune disease, are individuals exhibiting “a pre-clinical disease state”. The pre-disease state may be diagnosed based on developing symptoms, physical findings, laboratory test results, imaging results, and other findings that result in the individual meeting the diagnostic criteria for the autoimmune disease, and thus being formally diagnosed. Individuals with “pre-clinical disease” exhibit findings that suggest that the individual is in the process of developing the autoimmune disease, but do not exhibit findings, including the symptoms, clinical findings, laboratory findings, and/or imaging findings, etc. that are necessary to meet the diagnostic criteria for a formal diagnosis of the autoimmune disease. In some embodiments, individuals exhibiting a pre-clinical disease state possess a genetic variant or a combination of genetic variants that place them at increased risk for development of disease as compared to individuals who do not possess that genetic variant or that combination of genetic variants. In some embodiments, these individuals have laboratory results, or physical findings, or symptoms, or imaging findings that place them at increased risk for development of an autoimmune disease. In some embodiments, individuals with preclinical disease states are asymptomatic. In some embodiments, individuals with pre-clinical disease states exhibit increased or decreased levels of the expression of certain genes, certain proteins, autoimmune markers, metabolic markers, and other markers.

In certain embodiments, this invention is directed to the treatment of individuals with established autoimmune disease or disease associated with inflammation. The autoimmune disease can be diagnosed based on an individual that exhibits symptoms, signs, clinical features, laboratory test results, imaging test results, biomarker results, and other findings that enable a physician to formally diagnose that individual with the autoimmune disease, which findings can include the detection of pathogenic B cells activated by a cross-reactive antigenic peptide as disclosed herein.

In some embodiments, established autoimmune disease is an autoimmune disease for which an individual has had a formal diagnosis of the disease made by a physician for longer than 6 months. In established autoimmune disease, the signs or symptoms of disease may be more severe as compared to, for example, the symptoms for an individual diagnosed with early-stage autoimmune disease. In established autoimmune disease, the disease process may cause tissue or organ damage. As described herein, in certain embodiments, determination of inflammation in an individual with established disease can comprise analyzing the individual for the presence of at least one marker indicative of the presence of inflammation.

An autoimmune disease is considered a disease which exhibits clinical manifestations (abnormal clinical markers) such as visible inflammation including pain, swelling, warmth, and redness, and with respect to the present invention, will involve as a causative agent antigen-specific pathologic CD4+ T cells. Autoimmune diseases include without limitation autoimmune diseases, and may further include diseases with a specific T cell mediated component.

### EBV-Mediated Cancers

EBV is also associated with various non-malignant, premalignant, and malignant lymphoproliferative diseases such as Burkitt lymphoma, hemophagocytic lymphohistiocytosis, and Hodgkin's lymphoma; non-lymphoid malignancies such as gastric cancer and nasopharyngeal carcinoma; and conditions associated with human immunodeficiency virus such as hairy leukoplakia and central nervous system lymphomas. About 200,000 cancer cases per year are thought to be attributable to EBV.

### EBV-Mediated Infections

Infectious diseases associated with EBV infection include infectious mononucleosis and chronic active EBV infection. Most people are infected by EBV as children, when the disease produces few or no symptoms. In young adults, the disease often results in fever, sore throat, enlarged lymph nodes in the neck, and tiredness. Most people recover in two to four weeks; however, feeling tired may last for months. The liver or spleen may also become swollen, and in less than one percent of cases splenic rupture may occur. Chronic active EBV infection is actually classified as lymphoproliferative disorder. It is a rare and often fatal complication of EBV infection that most often occurs in children or adolescents of Asian or South American lineage, although cases in Hispanics, Europeans and Africans have been reported. Symptoms are fever, hepatitis, splenomegaly, and pancytopenia.

### Identification of a Cross-Reactive Antigenic Peptide

GlialCAM, also known in the art as hepaCAM is a glycoprotein containing an extracellular domain with 2 Ig-like loops, a transmembrane region and a cytoplasmic domain. GlialCAM is expressed at particularly high levels in the central nervous system (CNS). Functionally, glialCAM is involved in cell-extracellular matrix interactions and growth control of cancer cells, and is able to induce differentiation of glioblastoma cells. In cell signaling, GlialCAM directly interacts with F-actin and calveolin 1, and is capable of inducing senescence-like growth arrest via a p53/p21-dependent pathway. It acts as a chaperone for Aquaporin-4, which is the main autoantigen in the MS-related neuroinflammatory disorder neuromyelitis optica (NMO). GlialCAM can be proteolytically cleaved near the transmembrane region. The reference protein sequence may be found at Genbank, locus NP_689935. The mature protein is residues 34-416.

MKRERGALSR ASRALRLAPF VYLLLIQTDP LEGVNITSPV
RLIHGTVGKS ALLSVQYSST SSDRPVVKWQ LKRDKPVTVV
QSIGTEVIGT LRPDYRDRIR LFENGSLLLS DLQLADEGTY
EVEISITDDT FTGEKTINLT VDVPISRPQV LVASTTVLEL
SEAFTLNCSH ENGTKPSYTW LKDGKPLLND SRMLLSPDQK
VLTITRVLME DDDLYSCMVE NPISQGRSLP VKITVYRRSS
LYIILSTGGI FLLVTLVTVC ACWKPSKRKQ KKLEKQNSLE
YMDQNDDRLK PEADTLPRSG EQERKNPMAL YILKDKDSPE
TEENPAPEPR SATEPGPPGY SVSPAVPGRS PGLPIRSARR
YPRSPARSPA TGRTHSSPPR APSSPGRSRS ASRTLRTAGV
HIIREQDEAG PVEISA

EBNA1 is a major transcription factor of the Epstein-Barr virus. It plays an essential role in replication and partitioning of viral genomic DNA during latent viral infection. The sequence of EBNA1 may be accessed at public databases, for example at UniParc P03211-1.

MSDEGPGTGP GNGLGEKGDT SGPEGSGGSG PQRRGGDNHG
RGRGRGRGRG GGRPGAPGGS GSGPRHRDGV RRPQKRPSCI
GCKGTHGGTG AGAGAGGAGA GGAGAGGGAG AGGGAGGAGG
AGGAGAGGGA GAGGGAGGAG GAGAGGGAGA GGGAGGAGAG
GGAGGAGGAG AGGGAGAGGG AGGAGAGGGA GGAGGAGAGG
GAGAGGAGGA GGAGAGGAGA GGGAGGAGGA GAGGAGAGGA
GAGGAGAGGA GGAGAGGAGG AGAGGAGGAG AGGGAGGAGA
GGGAGGAGAG GAGGAGAGGA GGAGAGGAGG AGAGGGAGAG
GAGAGGGGRG RGGSGGRGRG GSGGRGRGGS GGRRGRGRER
ARGGSRERAR GRGRGRGEKR PRSPSSQSSS SGSPPRRPPP
GRRPFFHPVG EADYFEYHQE GGPDGEPDVP PGAIEQGPAD
DPGEGPSTGP RGQGDGGRRK KGGWFGKHRG QGGSNPKFEN
IAEGLRALLA RSHVERTTDE GTWVAGVFVY GGSKTSLYNL
RRGTALAIPQ CRLTPLSRLP FGMAPGPGPQ PGPLRESIVC
YFMVFLQTHI FAEVLKDAIK DLVMTKPAPT CNIRVTVCSF
DDGVDLPPWF PPMVEGAAAE GDDGDDGDEG GDGDEGEEGQ
E

As shown in the Examples, the peptide antigen thus identified may be a native peptide of the individual, or may be cross-reactive EBNA-1 peptide that activates B cells. The peptide is useful as a screening tool, and also finds use as a therapeutic agent to activate tolerance.

Peptides, including the cross-reactive peptides disclosed herein, usually comprise at least about 8 amino acids, at least about 9 amino acids, at least about 10 amino acids, at least about 11 amino acids, at least about 12 amino acids, at least about 13 amino acids, at least about 15 amino acids, or more, and may be from about 8 amino acids in length to about 40 amino acids in length, from about 8 to about 30 amino acids in length, from about 8 to about 25, from about 8 to about 20 amino acids in length, from about 8 to about 18 amino acids in length. A peptide may, for example, comprise the provided amino acid sequence of glialcam, including the epitope cross-reactive with EBNA-1, and may further include fusion polypeptides as known in the art in addition to the provided sequences, where the fusion partner is other than a native protein sequence. Peptides useful in this invention also include derivatives, variants, and biologically active fragments of naturally occurring peptides, and the like. The peptide may, for example, comprise 1 amino acid substitution, 2 amino acid substitutions, 3 amino acid substitutions. The peptide sequence may be a designed sequenced derived from mutagenesis in the diverse peptide library.

Peptides can be modified, e.g., joined to a wide variety of other oligopeptides or proteins for a variety of purposes. For example, post-translationally modified, for example by prenylation, acetylation, amidation, carboxylation, glycosylation, pegylation, etc. Such modifications can also include modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. In some embodiments, variants of the present invention include variants having phosphorylated amino acid residues, e.g. phosphotyrosine, phosphoserine, or phosphothreonine.

The ability of a peptide to modulate lymphocyte activity can be determined, for example, by the ability of the peptide to bind to pathogenic B cells or antibodies present in the peripheral blood, or CSF.

In some embodiments, a peptide is provided as a fusion protein, e.g., fused in frame with a second polypeptide. In some embodiments, the second polypeptide is capable of increasing the size of the fusion protein, e.g., so that the fusion protein will not be cleared from the circulation rapidly. In some other embodiments, the second polypeptide is part or whole of Fc region. In some other embodiments, the second polypeptide is any suitable polypeptide that is substantially similar to Fc, e.g., providing increased size and/or additional binding or interaction with Ig molecules. These fusion proteins can facilitate purification and show an increased half-life in vivo. Fusion proteins having disulfide-linked dimeric structures (due to the IgG) can also be more efficient in binding and neutralizing other molecules than the monomeric secreted protein or protein fragment alone.

In some other embodiments, peptide variants of the present invention include variants further modified to improve their resistance to proteolytic degradation or to optimize solubility properties or to render them more suitable as a therapeutic agent. For example, variants of the present invention further include analogs containing residues other than naturally occurring L-amino acids, e.g. D-amino acids or non-naturally occurring synthetic amino acids. D-amino acids may be substituted for some or all of the amino acid residues.

The polypeptides may be prepared by cell-free translation systems, or synthetic in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### Antibodies Specific for Cross-Reactive Peptides

Antibodies may be raised to the cross-reactive peptide(s), or may comprise a set of CDR sequences from the sequences provided in Table 3. As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (lgs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure.

The Fc region of naturally-occurring antibodies binds to elements of the complement system, and also to receptors on effector cells, including for example effector cells that mediate cytotoxicity, including specifically ADCP. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal.

In some embodiments, an antibody has constant region sequences that are characteristic of mouse, rabbit, primate, or human antibodies. In some embodiments, antibody sequence elements are humanized, primatized, chimeric, etc., as is known in the art.

Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc.), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE® s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload, e.g., a detectable moiety, a therapeutic moiety, a catalytic moiety, etc., or other pendant group [e.g., poly-ethylene glycol, etc.

Exemplary antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins.

In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv see Pluckthun, in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

There are five major classes of immunoglobulins: IgA, IgD, IgE, IgG, and IgM, and several of these can be further divided into subclasses (isotypes), e.g., IgG1, IgG2, IgG3, IgG4, IgA1, IgA2. The heavy-chain constant domains that correspond to the different classes of immunoglobulins are called a, d, e, g, and m, respectively. The subunit structures and three-dimensional configurations of different classes of immunoglobulins are well known. Engineered variants of immunoglobulin subclasses, including those that increase or decrease immune effector functions, half-life, or serum-stability, are also encompassed by this terminology.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a Kd (dissociation constant) of 10-5 M or less (e.g., 10-6 M or less, 10-7 M or less, 10-8 M or less, 10-9 M or less, 10-10 M or less, 10-11 M or less, 10-12 M or less, 10-13 M or less, 10-14 M or less, 10-15 M or less, or 10-16 M or less). “Affinity” refers to the strength of binding, increased binding affinity being correlated with a lower Kd.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

### Therapy and Diagnosis

Depletion of pathogenic B cells may comprise, for example, targeting antibodies to markers present on actively infected B cells, e.g. target pathogenic EBV-infected B cells, e.g. by therapies directed to one or more of cell-surface EBV proteins: BILF-1, LMP1 and LMP2. Antibodies may be conjugated to a cytotoxic agent, e.g. tubulin polymerization inhibitors, e.g. maytansinoids (maytansine), dolastatins, auristatin drug analogs, cryptophycin; duocarmycin derivatives, e.g. CC-1065 analogs, duocarmycin; enediyne antibiotics, e.g. esperamicin, calicheamicin; pyrrolobenzodiazepine (PBD); and the like. Other targeted agents include anti-B cell antibodies, e.g. anti-CD20 antibodies, e.g. rituximab; anti-CD19 antibodies; anti-CD38 antibodies, e.g. daratumumab; EBV-specific CAR T cells, and the like.

Inhibition of B cells with active EBV infection may utilize, for example, inhibition of specific tyrosine kinase proteins. Such inhibitor include, without limitation, BTK inhibitors. BTK signaling influences antigen presentation on B cells and is essential to the production of antibodies, proinflammatory cytokines and chemokines, and cell adhesion molecules. Examples of useful inhibitors include ibrutinib, evobrutinib, PRN2246 (SAR442168), and BIIB091.

Inhibition of active EBV infection may utilize small molecules that directly interfere with activating signaling cascades initiated by EBV-encoded proteins, e.g. LMP-1 and LMP-2.

Therapeutic entities are often administered as pharmaceutical compositions comprising an active therapeutic agent and a other pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Also provided are combination therapy methods, where the combination may provide for additive or synergistic benefits. Combinations of a peptide or antibody may be obtained with a second agent selected from one or more of the general classes of drugs commonly used in the non-antigen specific treatment of autoimmune disease, which include corticosteroids and disease modifying drugs; or from an antigen-specific agent. Corticosteroids, e.g. prednisone, methylpredisone, prednisolone, solumedrol, etc. have both anti-inflammatory and immuno activity. They can be given systemically or can be injected locally. Corticosteroids are useful in early disease as temporary adjunctive therapy while waiting for disease modifying agents to exert their effects. Corticosteroids are also useful as chronic adjunctive therapy in patients with severe disease.

Certain compounds are known to activate EBV in B cells and therefore induce expression of BILF-1, and other lytic/activated proteins, as well as possibly LMP-1, LMP-2. These compounds include but are not restricted to decitabine, sodium butyrate, bortezomib, and compounds described by Tikhmyanova et al. (Bioorg Med Chem Lett., 2014 and 2019). These compounds could be used in conjunction with anti-EBV antibodies and other compounds to increase expression of EBV-encoded target molecules for depletion of EBV-infected B cells.

Disease modifying drugs are also useful in combined therapy. These agents include methotrexate, leflunomide, etanercept, infliximab, adalimumab, anakinra, rituximab, CTLA4-Ig (abatacept), antimalarials, gold salts, sulfasalazine, d-penicillamine, cyclosporin A, cyclophosphamide azathioprine; and the like. Treatments for MS may include interferon β, Copaxone, and anti-VLA4, which reduce relapse rate. MS is also treated with immunosuppressive agents including methylprednisolone, other steroids, methotrexate, cladribine and cyclophosphamide.

Combination therapies may be sequentially staged, provided in a co-administration formulation, or concomitant administration during the same time period. “Concomitant administration” of a known therapeutic drug with a pharmaceutical composition of the present invention means administration of the drug and peptide at such time that both the known drug and the composition of the present invention will have a therapeutic effect. Such concomitant administration may involve concurrent (i.e. at the same time), prior, or subsequent administration of the drug with respect to the administration of a compound of the invention. A person of ordinary skill in the art would have no difficulty determining the appropriate timing, sequence and dosages of administration for particular drugs and compositions of the present invention.

Drug, peptides, antibodies, etc. can serve as the active ingredient in pharmaceutical compositions formulated for the treatment of various disorders as described above. The active ingredient is present in a therapeutically effective amount, i.e., an amount sufficient when administered to treat a disease or medical condition mediated thereby, in particular by reducing the activity of inflammatory lymphocytes. The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

Complexes with various well-known compounds can be used to enhance the in vivo stability of a drug or polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. The dosage of the active ingredient typically lies within a range of circulating concentrations that include the ED50 with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal method.

Formulations suitable for parenteral administration, such as, for example, by intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are preferably sterile. To the extent that a given compound must be synthesized prior to use, the resulting product is preferably substantially free of any potentially toxic agents, such as any endotoxins, which may be present during the synthesis or purification process. Compositions for parental administration are also preferably sterile, substantially isotonic and made under GMP conditions.

The compositions may be administered in a single dose, or in multiple doses, usually multiple doses over a period of time, e.g. daily, every-other day, weekly, semi-weekly, monthly etc. for a period of time sufficient to reduce severity of the inflammatory disease, which may comprise 1, 2, 3, 4, 6, 10, or more doses.

Determining a therapeutically or prophylactically effective amount can be done based on animal data using routine computational methods. In one embodiment, the therapeutically or prophylactically effective amount contains between about 0.1 mg and about 1 g of protein. In another embodiment, the effective amount contains between about 1 mg and about 100 mg of protein. In a further embodiment, the effective amount contains between about 10 mg and about 50 mg of the protein. The effective dose will depend at least in part on the route of administration. The dose may be from about 0.1 μg/kg patient weight; about 1 μg/kg; about 100 μg/kg; to about 10 mg/kg.

In methods of use, an effective dose of an agent of the invention is administered alone, or combined with additional active agents for the treatment of a condition as listed above. The effective dose may be from about 1 ng/kg weight, 10 ng/kg weight, 100 ng/kg weight, 1 μg/kg weight, 10 μg/kg weight, 25 μg/kg weight, 50 μg/kg weight, 100 μg/kg weight, 250 μg/kg weight, 500 μg/kg weight, 750 μg/kg weight, 1 mg/kg weight, 5 mg/kg weight, 10 mg/kg weight, 25 mg/kg weight, 50 mg/kg weight, 75 mg/kg weight, 100 mg/kg weight, 250 mg/kg weight, 500 mg/kg weight, 750 mg/kg weight, and the like. The dosage may be administered multiple times as needed, e.g. every 4 hours, every 6 hours, every 8 hours, every 12 hours, every 18 hours, daily, every 2 days, every 3 days, weekly, and the like. The dosage may be administered orally.

The compositions can be administered in a single dose, or in multiple doses, usually multiple doses over a period of time, e.g. daily, every-other day, weekly, semi-weekly, monthly etc. for a period of time sufficient to reduce severity of the inflammatory disease, which can comprise 1, 2, 3, 4, 6, 10, or more doses.

Determining a therapeutically or prophylactically effective amount of an agent according to the present methods can be done based on animal data using routine computational methods. The effective dose will depend at least in part on the route of administration.

The cross-reactive peptides are also useful in methods of characterizing the immune profile of an individual, particularly for determining the presence of pathogenic B cells having specificity for these peptides in an individual suspected of having MS or related inflammatory conditions. The methods can comprise contacting a sample comprising B cells from the individual with an immunogenic, cross-reactive peptide, and determining the presence of a B cell or antibody response to the peptide. The sample may be any biological sample that comprises B cells or antibodies, including peripheral blood, lymph node samples, CSF, and the like. The response can be determined by direct binding assays, by determining the presence of B cells associated with specificity to these peptide antigens, by determining the presence of EBV activation markers on B cells, by frequency determination; and the like as known in the art.

### Antigen-Specific Immunotherapy

Antigen-specific immunotherapy aims to take advantage of tolerization, immune deviation and the induction of Tregs in order to promote autoantigen-specific tolerance. Autoimmune diseases are potentially be treated by eliminating pathogenic cells that are specific for autoantigens or by blocking the immune response directed by autoantigen-specific cells. Another method to induce immunological changes is by manipulation of dendritic cells (DCs). DCs are essential to the induction phase of the immune response and are therefore critically important in determining whether a response toward an antigen will be inflammation or tolerance. DCs can influence if naïve cells will undergo deletion, anergy, or differentiation. DC responses to a specific antigen are influenced by the tissue environment and innate stimuli associated with that antigen. Therapies may target DCs to induce tolerance.

For example, the cross-reactive EBNA-1 epitope or glialcam epitope may be administered via a tolerogenic route, e.g. by oral or nasal administration of soluble or oligomerized peptides. Alternatively the cross-reactive peptide can be used as the basis for an altered peptide ligand (APLs). Altered peptide ligands are analogues derived from an antigenic peptide that comprise amino acid substitutions at contact residues, e.g. a substitution of 1, 2 3 amino acids. Altered peptide ligands can specifically antagonize and inhibit activation induced by the cognate antigenic peptide. APLs compete with the native peptide for binding but bind with lower affinity, and can thereby function as antagonists or partial agonists.

In some embodiments a peptide is formulated for immunization to generate an antigen-specific tolerance, e.g. by subcutaneous or oral administration of a cross-reactive peptide(s). In some embodiments a cross-reactive peptide is formulated for trans-dermal delivery. In some embodiments, a method of inducing immune tolerance comprises trans-dermal administration of cross-reactive peptide(s) this formulated. An effective dose may be a low dose, e.g. a dose of less than about 5 mg, less than about 2.5 mg, less than about 1 mg, less than about 500 μg, less than about 100 μg. In some embodiments a cross-reactive peptide is encapsulated into mannosylated liposomes to enhance enhanced the uptake of the peptides by dendritic cells.

As an alternative to peptide vaccination, DNA vaccines can be formulated in a tolerizing vector of genetically engineered DNA that encodes one or more of the cross-reactive peptides disclosed herein. A tolerizing vector can be formulated and administered by intramuscular injection, for example in a plasmid backbone modified in such a way that it could lead to favorable immunological changes in patients with MS, e.g. reduction in the number of immunostimulatory CpG motifs and increase in the number of immunoinhibitory GpG motifs). A lower dose may be preferred, e.g. a dose of less than 5 mg, e.g. a dose of less than about 5 mg, less than about 2.5 mg, less than about 1 mg, less than about 500 μg, less than about 100 μg.

DNA vectors, for example as described in U.S. Pat. No. 10,098,935 have been shown to provide for tolerization (i.e., induction of antigen-specific tolerance). Such a vector is referred to as a tolerizing vector. The vector can be administered, for example, by local injection, including intramuscular injection, where the vector encodes a cross-reactive adenovirus peptide or protein comprising the peptide, and further comprises a promoter sequence operably linked the nucleic acid sequence; and a DNA backbone, linked to the promoter sequence and the nucleic acid sequence, comprising 4 or fewer immunostimulatory CpG motifs. The cross-reactive peptide may be modified by 1, 2, 3, or more amino acid residues to be altered from the naturally occurring polypeptide.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

### Kits

Also provided are kits for use in the subject methods. The subject kits include any combination of components and compositions for performing the subject methods. In some embodiments, a kit can include one or more of the following: a cross-reactive EBNA-1 or glialcam peptide for determining the presence of reactive cells or antibodies; a cross-reactive EBNA-1 or glialcam peptide for inducing tolerance; reagents for detecting EBV-driven pathogenic B cells, a vaccine delivery device, a suitable buffer and any combination thereof.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example 1

### The B Cell Repertoire in Multiple Sclerosis Reveals Molecular Mimicry Between EBNA1 and GlialCAM

Multiple sclerosis (MS) is a heterogenous autoimmune disease where autoreactive T and B lymphocytes attack the myelin sheaths of the central nervous system (CNS). Lymphocytes in the cerebro-spinal fluid (CSF) are directly involved in inflammation of the adjacent CNS and accessible to investigation. Intrathecal B cells secrete oligoclonal immunoglobulin, distinct from the immunoglobulin in the circulation, suggesting continual activation of specific plasmablasts by a private antigen. However, phenotype, function, and antigen-specificity of autoreactive B cells are not well understood. Molecular mimicry between viruses and CNS proteins has been proposed as a pathogenic factor for MS, but identification of cross-reactive antigens has been challenging. Here we describe phenotypic differences of B cell phenotypes in blood and CSF of MS patients, which implicate plasmablasts in MS pathogenesis. Sequencing of the paired-chain antibody repertoire of over 15,000 single-cell sorted plasmablasts in CSF and blood of 9 MS patients revealed ongoing intrathecal somatic hypermutation and antigen-specific clonal expansion of plasmablasts in the CSF. We tested over 140 potentially pathogenic antibodies derived from CSF plasmablasts against a spectrum of viruses implicated in MS pathogenesis. We identified a CSF-plasmablast derived antibody that binds the Epstein-Barr Virus (EBV) transcription factor EBNA1 and cross-reacts to the glial cellular adhesion molecule GlialCAM. Immunization of mice with the identified EBNA1 epitope aggravates the mouse model of MS. ˜ 15% of MS patients carry antibodies that cross-react to EBNA1 and GlialCAM. Together, our results suggest that EBNA1-reactive antibodies EBV can cross-react with the CNS-specific membrane protein GlialCAM and induce neuroinflammation, thereby exacerbating MS.

Here we show that a large fraction of B cells in the CSF are activated plasmablasts (PB) that are distinct from PB in blood with regard to activation and trafficking markers. To understand their antigen-specificity, we sequenced the single-cell paired-chain BCR repertoire of more than 1600 B cells from CSF and over 13,000 PB from blood of 9 MS patients and found substantially higher clonality and skewed IGHV gene usage in the CSF, indicative of ongoing intrathecal somatic hypermutation and antigen-specific proliferation. 148 BCR sequences representative of large clonal expansions were expressed as recombinant monoclonal antibodies (mABs) and tested for reactivity to viral proteins and peptide, with an emphasis on Epstein Barr Virus (EBV).

The CSF-derived mAB MS39p2w174 was discovered, which binds to EBNA1 within a region previously described to be associated with higher serum reactivity in MS patients (AA386-405). MS39p2w174 cross-reacts to the glial cell adhesion molecule GlialCAM, a type 1 membrane protein expressed on oligodendrocytes and astrocytes, in particular at the astrocytic perivascular endfeet that maintain blood brain barrier integrity. GlialCAM aids the correct expression of aquaporin-4, an important B cell antigen in neuromyelitis optica. In addition, it enables cell-cell contacts by homo-oligomerization and is indispensable for glial chloride and water homeostasis as a beta-subunit of the MLC1 and an auxiliary subunit of CLC2. We mapped the EBNA1 and GlialCAM epitopes in detail, including a 2.5 Å crystal structure of the mAB/EBNA1 peptide complex. We can show that the unmutated germline sequence of MS39p2w174 is already prone to bind EBNA1, while additional mutations increase affinity to GlialCAM. Interaction with GlialCAM is facilitated by a serine phosphorylation N-terminal of the central epitope. Similar antibodies against GlialCAM are generated in mice upon immunization with EBNA1 AA386-405, which aggravates the mouse model of MS, experimental autoimmune encephalomyelitis (EAE). Elevated anti-GlialCAM serum reactivity was observed in MS patients in comparison to healthy individuals.

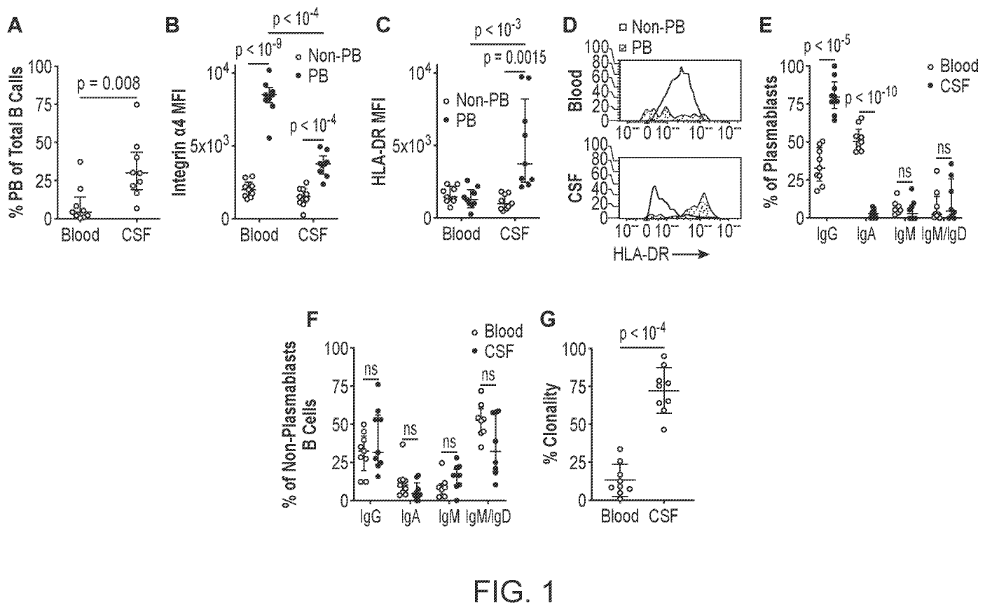

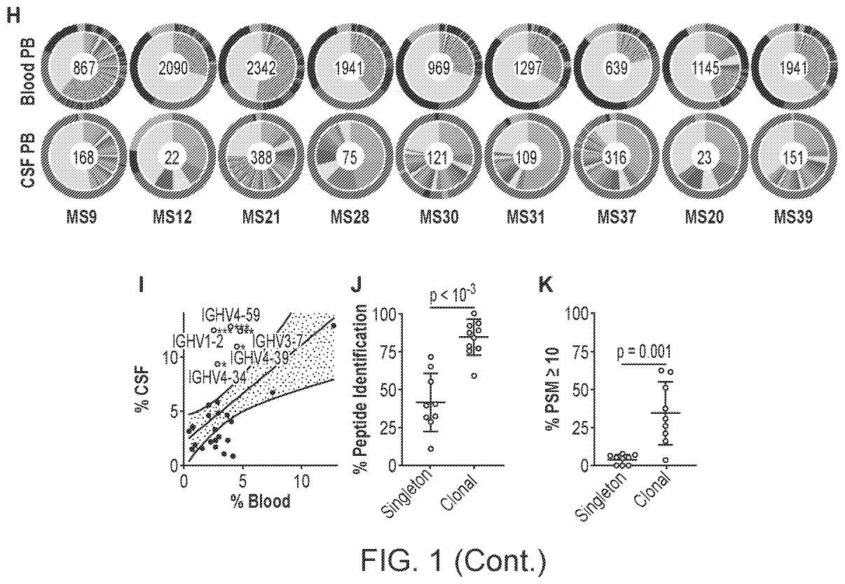

IgG+ plasmablasts dominate the CSF B cell compartment in MS patients. CSF and blood samples were obtained from 9 MS patients during the initial onset of disease (clinically isolated syndrome, CIS, n=5) or an acute episode of relapsing-remitting MS (RRMS, n=4) (table 1) and B cells were sorted by flow cytometry. Approximately 30% of B cells in the CSF exhibit an activated plasmablast (PB) phenotype (CD19+CD20low CD27+CD38+(PB), median: 29.8%, SD: 20), while only a small fraction of ˜4% of B cells in the blood are PB (median: 4.1%, SD: 12.2; FIG. 1A). Conversely, naïve B cells are diminished in the CSF, while unswitched and switched memory as well as double negative B cell numbers are comparable in blood and CSF. Profound phenotypic differences between blood and CSF were detected in PB but not in non-PB B cells: (i) Blood PB express high levels of the trafficking receptor a4 integrin, whereas PB in the CSF abate α4 over time (FIG. 1B). (ii) PB in the CSF express higher levels of HLA-DR than their blood-derived counterparts (FIG. 1C,D), emphasizing their role in antigen presentation in the CSF and CNS. Similar differences were not seen in non-PB B cells. Of note, HLA-DR expression was independent of the HLA-DRB1*15:01 genotype (HLA-DR15) (table 1 and FIG. 1E,F). (iii) The predominant immunoglobulin (lg) class within the PB compartment in CSF is IgG, while both IgA and IgG are the main classes in blood-derived PB (FIG. 1E). In contrast, non-PB B cells express similar Ig-classes in CSF and blood (FIG. 1F). These differences suggest an elevated pathogenic role of PB in MS.

The CSF PB immune repertoire is highly clonal and skewed. To gain a comprehensive overview of the intrathecal antigen-specific B cell response in MS, we sorted single PB from blood and single B cells from CSF of MS patients by flow cytometry and sequenced full-length paired heavy-chain (HC) and light-chain (LC) VDJ regions using our in-house plate-based single-cell sequencing technology. 13,231 paired sequences from blood PB and 1,689 from CSF B cells passed filter thresholds. In comparison to the repertoire of blood PB, the repertoire of CSF PB is significantly more clonal and largely dominated by IgG (FIG. 1G, H), suggesting antigen-specific proliferation of a few clones. Not surprisingly, non-PB B cells in the CSF are less clonal and express IgA and IgM more frequently than PB. While mutation counts in IGHV and IGLV genes did not differ significantly between PB in blood and CSF, HC-CDR3 lengths are on average 0.94 amino acids longer in CSF PB, indicating ongoing intrathecal somatic hypermutation. The repertoire in the CSF is skewed towards more preferential usage of 5 IGHV chains: IGHV4-59, IGHV4-39, IGHV4-34, IGHV1-2, and IGHV3-7 (FIG. 1I), which for the most part is in line with previous reports, and indicates that a select group of MS-related antigens in the CSF drive PB survival and proliferation in the CSF, with a few distinct immunoglobulin germline genes being predestined to bind them.

Clonal PB are the main source of oligoclonal bands. We hypothesized that clonally expanded PB are the main source of intrathecal oligoclonal bands (OCB) in MS. We purified immunoglobulin from CSF samples and sequenced the variable region amino acid sequences by mass spectrometry. As expected, clonally sequences were readily identified, whereas only fourty percent of singleton sequences detected 39.6% (FIG. 1J). Highly abundant immunoglobulins, which were identified with ten or more peptide-spectral matches (PSM>10) likely correlate with oligoclonal bands. These sequences aligned almost exclusively to clonally expanded B cell sequences (FIG. 1K), suggesting that they are the source of the oligoclonal bands. This correlation holds true for PB, which are more clonal than non-PB B cells. Taken together, clonal PB are likely the main source of antibodies and OCB in the CSF.

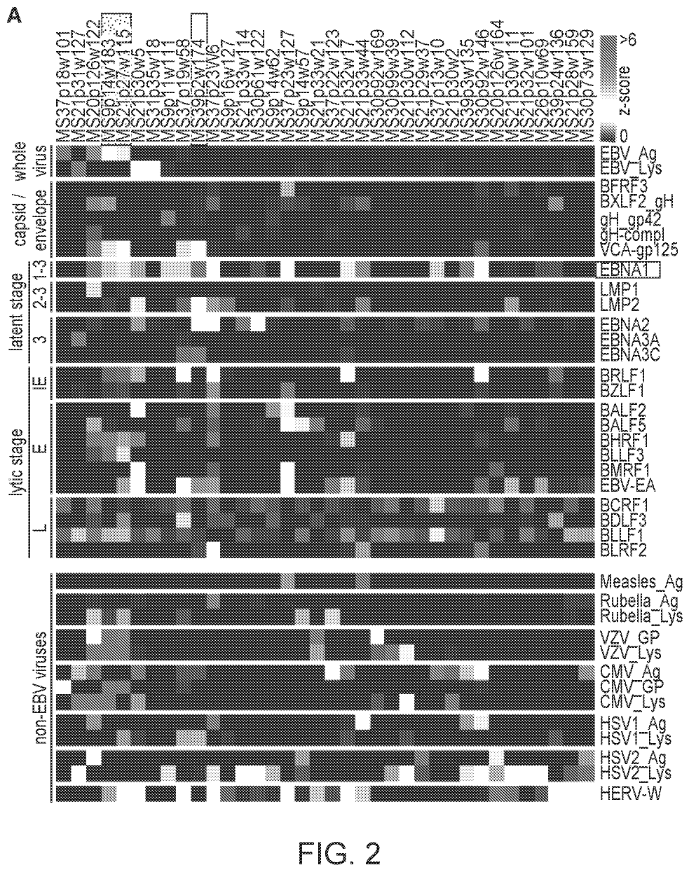

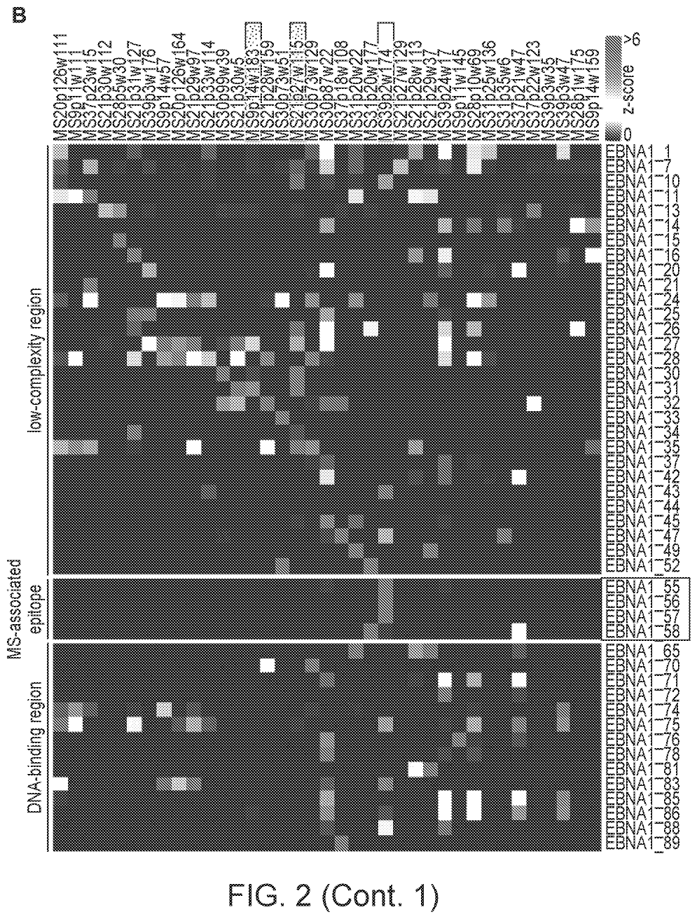

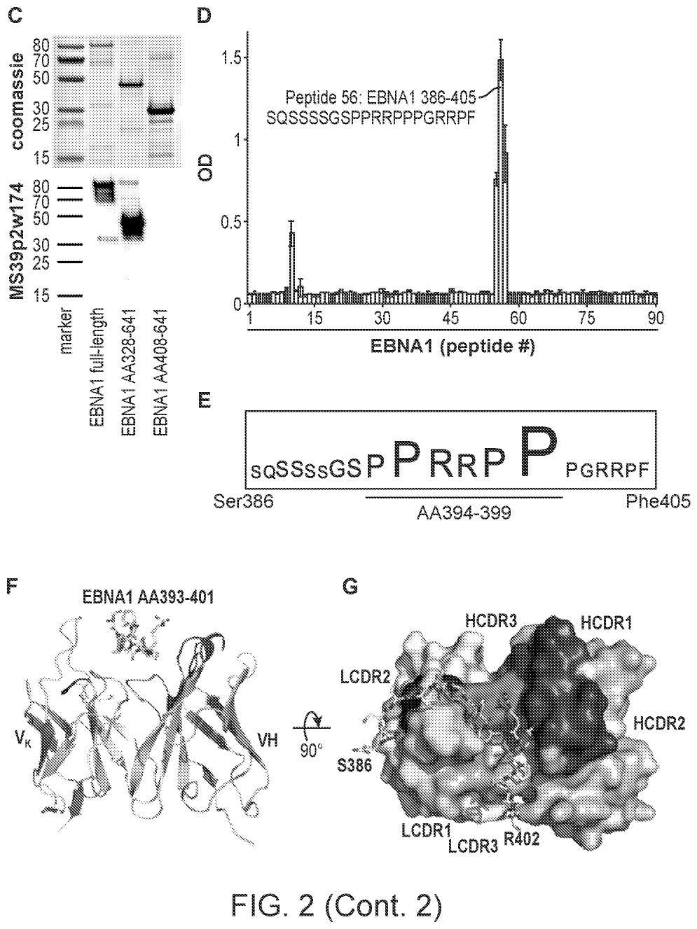

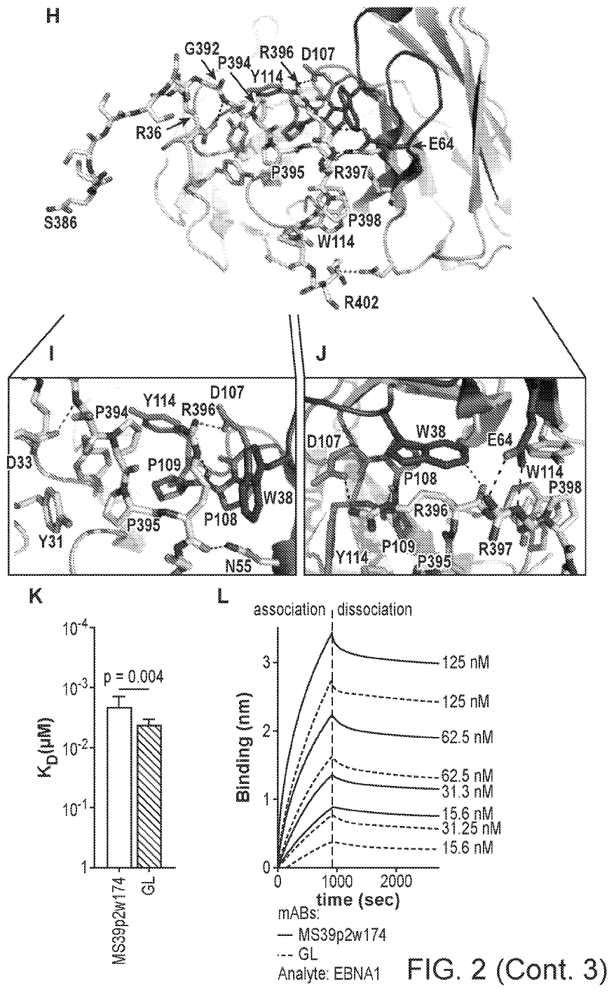

CSF-derived monoclonal antibodies bind EBV antigens. A total of 148 sequences from the CSF repertoires were selected for recombinant expression, each one representative of a major clonal expansion. To test anti-viral reactivities of the selected mABs, they were probed on a planar protein microarray representing EBV lysates, 23 recombinant latent and lytic EBV proteins, 240 peptides spanning four prominent EBV proteins, as well as 7 lysates of other MS-associated viruses, including measles, rubella, and varicella-zoster virus (VZV)³⁰ (FIG. 2A,B). One-third of the expressed mABs bound to EBV proteins and peptides and ˜20% to other viruses, in particular to VZV and CMV (FIG. 2A). Interestingly, half of the VZV-reactive antibodies cross-reacted to CMV and EBV, indicative of broader antigens common to herpes viruses.

Interestingly, we found mABs in 6 out of 9 patients that bound the transcription factor EBNA1 (FIG. 2A), and mABs binding to EBNA1 peptides in 8 out of 9 patients (FIG. 2B). Anti-EBNA1-reactivity has been implicated in MS pathogenesis and the region AA365-425 (“MS-associated epitope” in FIG. 2B) is known to elicit a stronger antibody response in MS patients than in healthy individuals. Protein and peptide arrays revealed that our mAB MS39p2w174 binds EBNA1 within this region (AA386-405, FIG. 2B). The interaction was verified by western blot analysis using full-length and truncated EBNA1 proteins (FIG. 2C) and ELISA-based peptide scans spanning full-length EBNA1 (20mer peptides, 13AA overlap, FIG. 2D). Alanine-scanning determined the proline-rich region AA394-399 to be the central epitope (FIG. 2E). Taken together, we identified multiple mABs directed against EBV and in particular MS39p2w174, which binds a well-described MS-associated epitope of EBNA1.

Crystal structure reveals key residues of mAB-EBNA1 interaction. While the presence of antibodies against the broader EBNA1 region AA365-425 is well established in MS patients, their relevance to MS pathology has remained elusive. Efforts to model its structure has done little to understand the epitopes functional properties. To understand its immunogenicity and impact on MS pathology in detail, we solved the crystal structure of MS39p2w174 in complex with EBNA1 AA386-405 at a resolution of 2.5 Å (FIG. 2F-J, PDB ID: 7K7R). It confirmed close interactions of the peptide residues P394-P398 with all complementary determining regions (CDRs) but the very short LCDR2. Residues Tyr31 and Tyr38 on LCDR1 together with Trp38 on HCDR1 and Pro108, Pro109, and Tyr114 on HCDR3 create a hydrophobic cage for the peptide's first two prolines Pro394 and Pro395 and the proximal side chain of Arg396 (FIG. 2H-J). The C-terminal end of the antibody binding groove is wider and Pro398 is carried by a large aromatic tryptophan residue (Trp114 in HCDR1) on the bottom of the groove (FIG. 2G,H,J). The central arginines Arg395 and Arg396 engage in close polar interactions (<3.1 Å) with residues on HCDR2, HCDR3, and HC framework region 2. Contrary to the results of our alanine scan (FIG. 2E), Pro399 does not appear to interact directly with antibody side chains, and we assume that alanine at position 399 disrupts the conformation of the three prolines Pro398-Pro400 causing steric hindrance within the binding pocket.

The encoding IGHV gene of MS39p2w174 is IGHV3-7, one of the IGHV chains over-represented in CSF (FIG. 1I). Interestingly, all but one of the residues that directly interact with EBNA1 are unmutated germline (GL) residues (IGHV3-7, IGHJ4, IGKV2-30, IGKJ1). We therefore hypothesized that the unmutated ancestor of MS39p2w174 might have an inert propensity to bind EBNA1 AA386-405. Indeed, we could show that GL binds to EBNA1 with only slightly lower affinity than MS39p2w174 (K_{D} MS39p2w174: 1.99 nM, GL: 4.19 nM) (FIG. 2K,L).

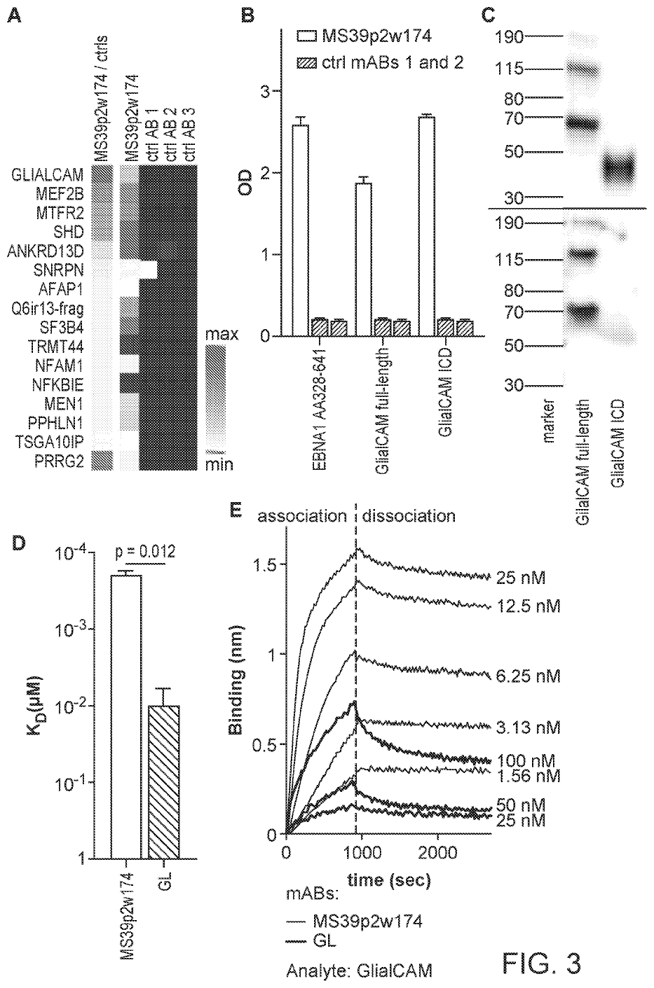

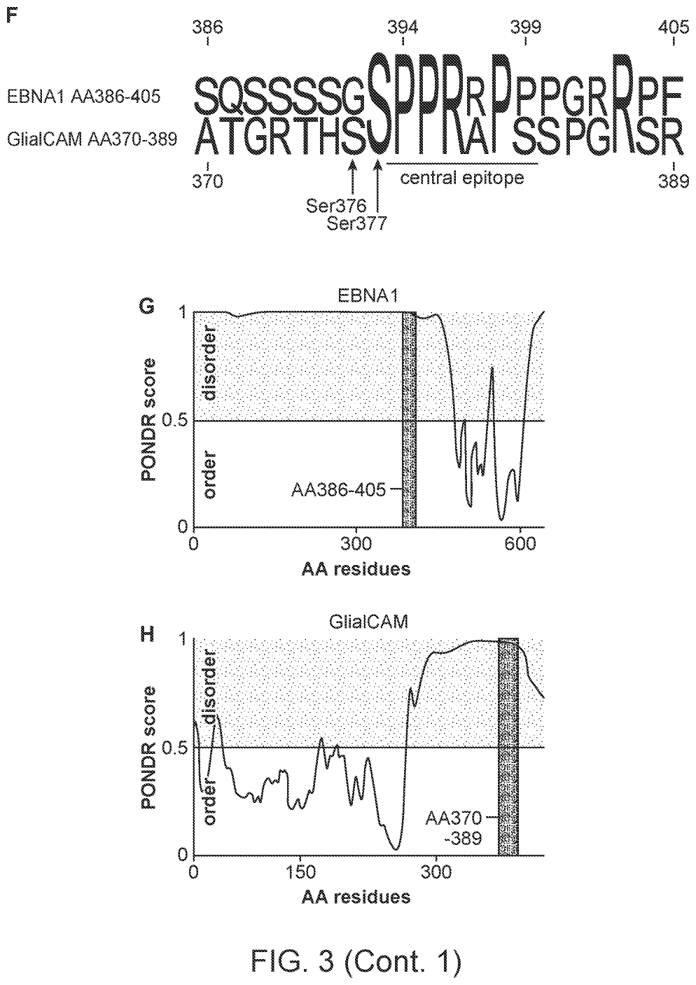

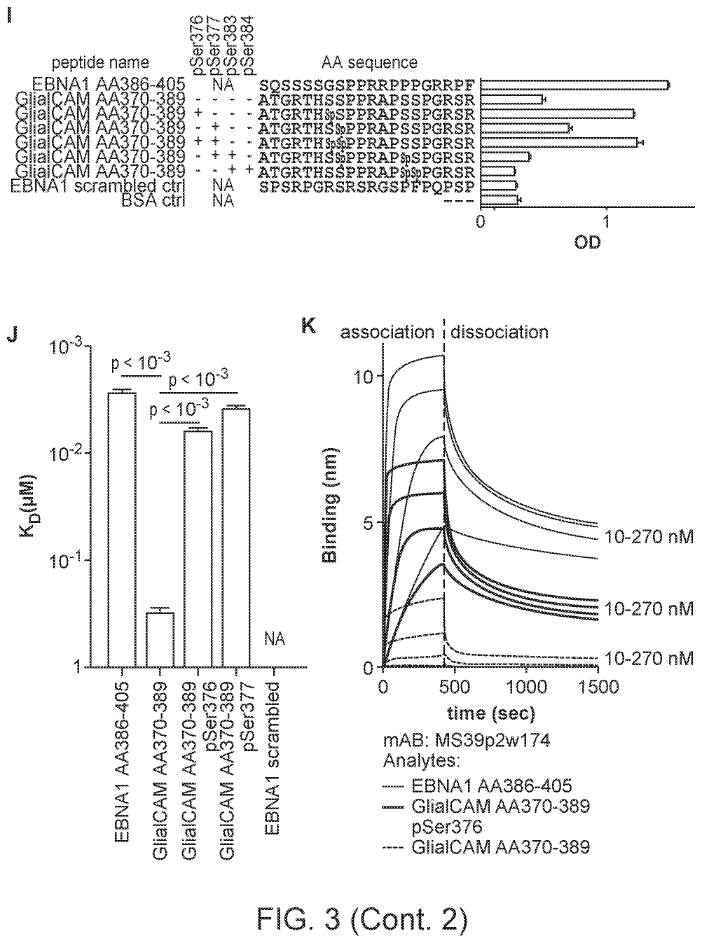

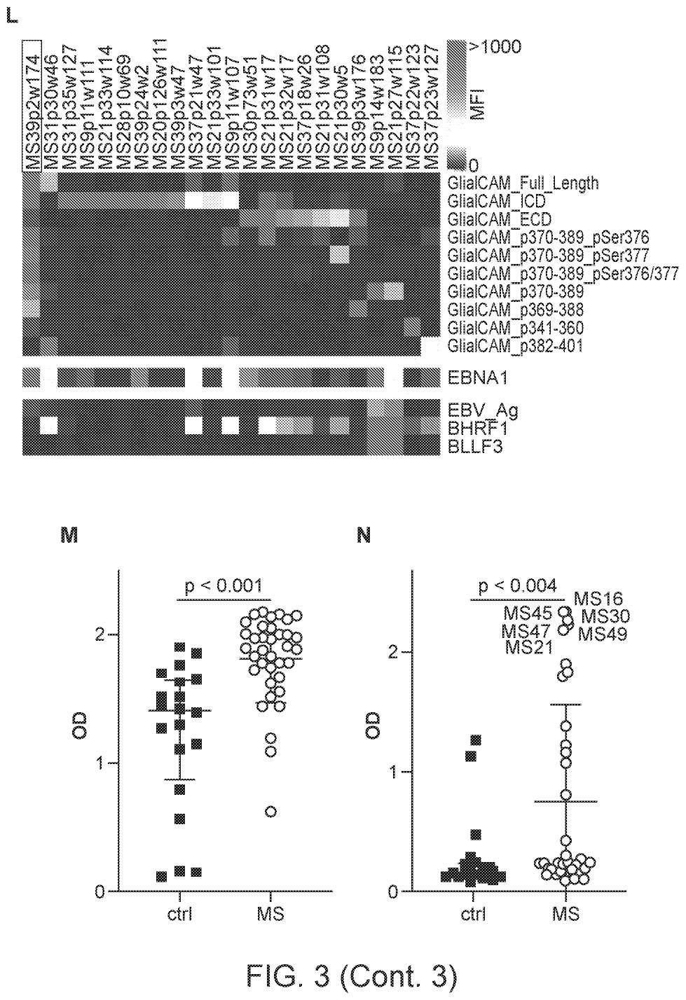

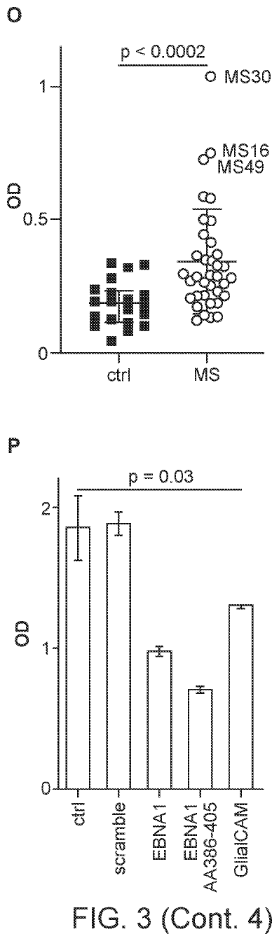

Molecular mimicry between EBNA1 AA386-405 and GlialCAM. Studying a mAB as opposed to patient-derived sera and CSF samples allows for direct identification of molecular mimicry with human proteins. We probed mAB MS39p2w174 on a HuProt protein microarray, which represents >20,000 proteins spanning the entire human proteome. Glial cell adhesion molecule (GlialCAM) was identified as the top binding partner to MS39p2w174 (FIG. 3A). GlialCAM is a cell adhesion molecule that is almost exclusively expressed in the CNS (www.proteinatlas.org), mainly in astrocytes and oligodendrocytes. In multiple sclerosis, it has been found to be decreased in acute and chronic MS plaques, but elevated in chronic-active plaques. GlialCAM AA337-385 was identified as a binding partner for MS39p2w174 on a 49mer phage display representing the whole human proteome (356 out of 10⁵ reads, only identified by MS39p2w174 in a set of 300 mABs). Binding of MS39p2w174 to the intracellular domain (ICD, AA262-416) of GlialCAM was confirmed on ELISA (FIG. 3B) and western blot (FIG. 3C). Affinity measurements with bio-layer interferometry revealed higher affinity of MS39p2w174 to GlialCAM (K_{D}: 190 PM) vs. EBNA1 (K_{D}: 1.99 nM). This is in contrast to the unmutated GL mAB, which binds GlialCAM with lower affinity (K_{D} GlialCAM: 10.46 nM, K_{D} EBNA1: 4.19 nM) (FIG. 2K,L and FIG. 3D,E). Evidently, while GL harbors a propensity to bind to EBNA1, somatic hypermutation during development of MS39p2w174 has increased its affinity to the CNS mimic GlialCAM by 2 orders of magnitude.

Phosphorylation at GlialCAM Ser376 enables binding of MS39p2w174. The EBNA1 epitope AA386-405 is located between the protein's long N-terminal Gly-Ala-rich low-complexity region (AA90-380) and its highly structured DNA-binding domain (AA: 461-607, PDB: 1B3T). On GlialCAM, the above-mentioned region AA337-385 is located at the C-terminal end of the ICD and contains a proline-rich region that closely resembles the central epitope of EBNA1 (FIG. 3F). MS39p2w174 detects both proteins on western blots under denaturing conditions (FIG. 2C, FIG. 3C), suggesting linear epitopes for both targets. This is in line with predictions that both epitopes are located in intrinsically disordered regions of the respective protein (FIG. 3G, H). However, while MS39p2w174 binds the EBNA1 peptide AA386-405 with high affinity (K_{D}: 2.67 nM), its affinity to GlialCAM peptide AA370-389 is drastically lower (K_{D}: 302 nM). As the intracellular domain of GlialCAM is heavily phosphorylated, and post-translational modifications often determine antibody-antigen interactions, we tested if phosphorylation at one of the 4 serine residues surrounding the central epitope region (residues Ser376, 377, 383, and 384) could increase binding affinity of MS39p2w174 to GlialCAM AA370-389. Indeed, phosphorylation at Ser376 facilitates MS39p2w174 interaction with the peptide (K_{D}: 6.1 nM) and additional phosphorylation of Ser377 further enhances binding affinity (K_{D}: 3.73 nM) (FIG. 3I-K). In contrast, citrullination of arginine residues Arg373, 380, and 387 did not alter peptide binding to MS39p2w174. The important residue Arg397 in EBNA1 AA386-405, which engages in 2 hydrogen-bonds with Glu64 at HCDR2 (FIG. 2 H,J) is replaced with alanine in GlialCAM AA370-389 (Ala381) (FIG. 3F), which explains the decreased binding affinity between MS39p2w174 and GlialCAM peptide. Phosphorylation at position 376 likely enables binding by adding new polar interactions to the proximal LC, possibly with Arg36, a positively charged residue that is mutated from asparagine in GL (FIG. 2H).

MS anti-GlialCAM IgG titers are elevated in MS patients. To test if the observed anti-GlialCAM reactivity of MS39p2w174 is part of a broader phenomenon, we tested our remaining 147 mABs for reactivity against GlialCAM protein and the broader region AA315-395. We found 10 additional mABs that bound the ICD and 7 that bound the extracellular domain (ECD) (FIG. 3L). Two mABs, MS9p14w183 and MS21p27w115 bound unphosphorylated EBNA1 AA370-389. Interestingly, both also cross-reacted with EBNA1 as well as with the two early lytic EBV proteins BHRF1 and BLLF3 (FIG. 3L). This shows that MS39p2w174 is not an isolated phenomenon. Albeit we did not identify another mAB in our collection with the exact same characteristics, antibodies against several GlialCAM epitopes were prevalent in the majority of patients.

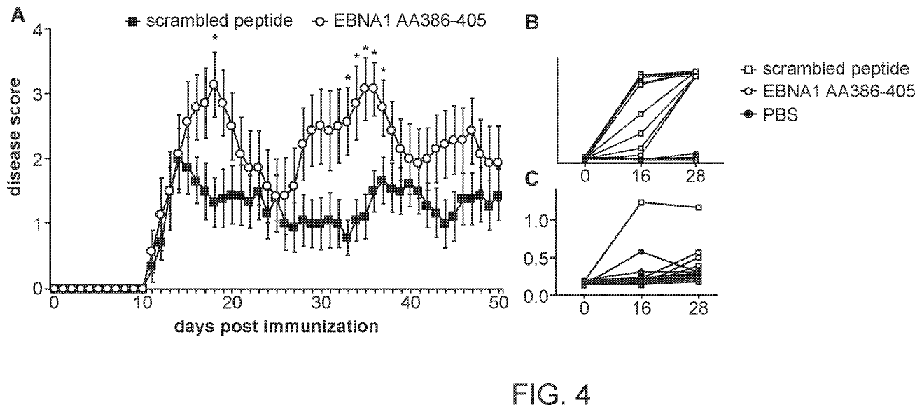

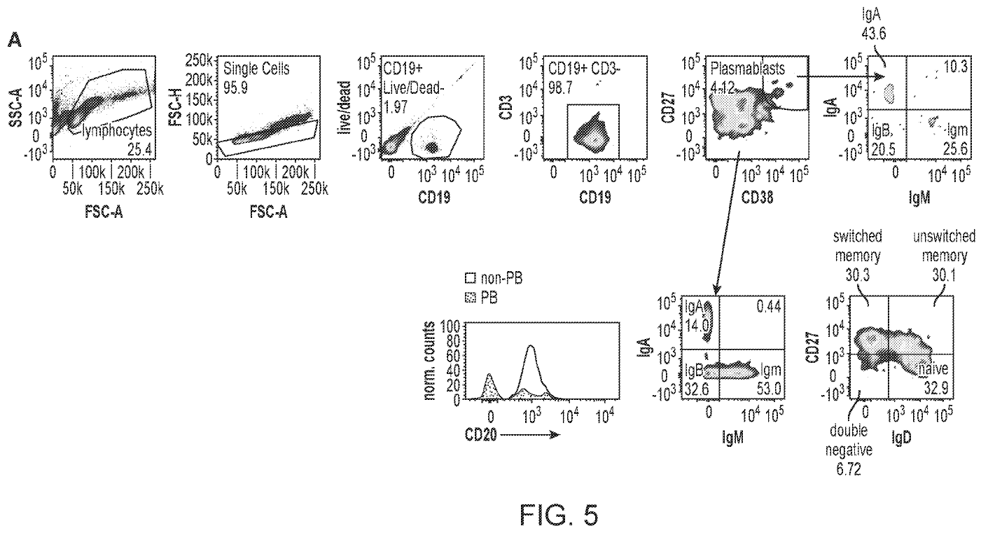

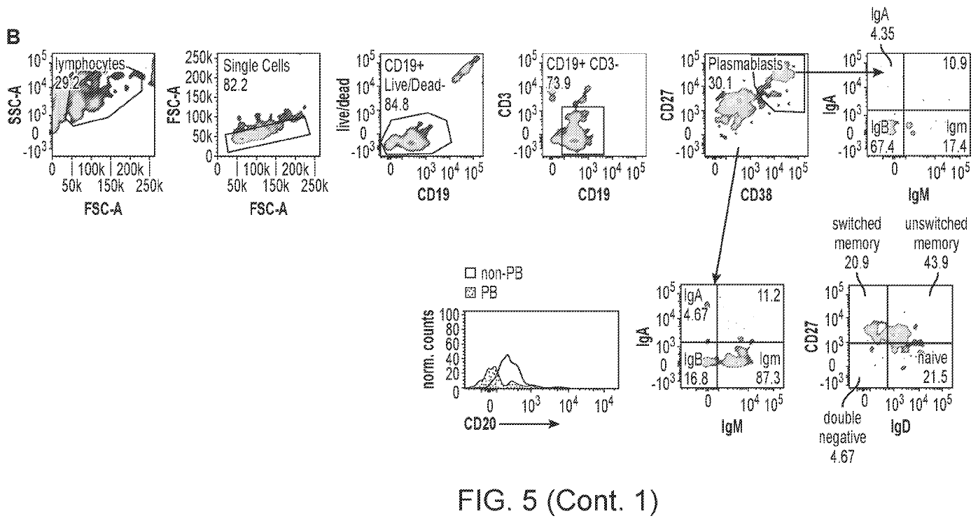

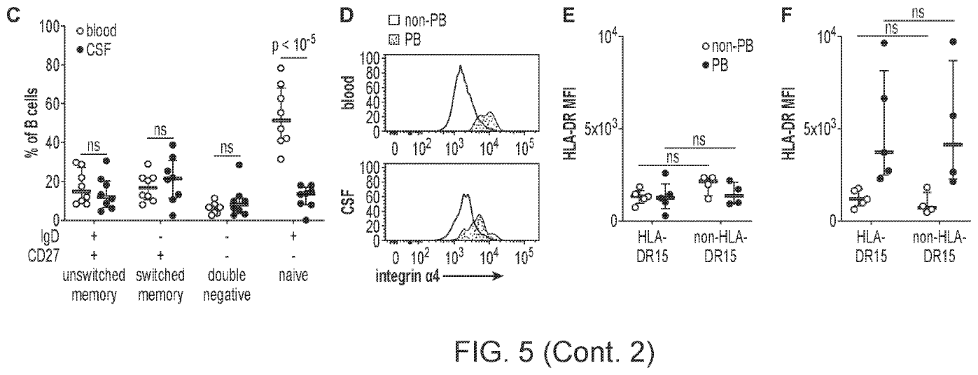

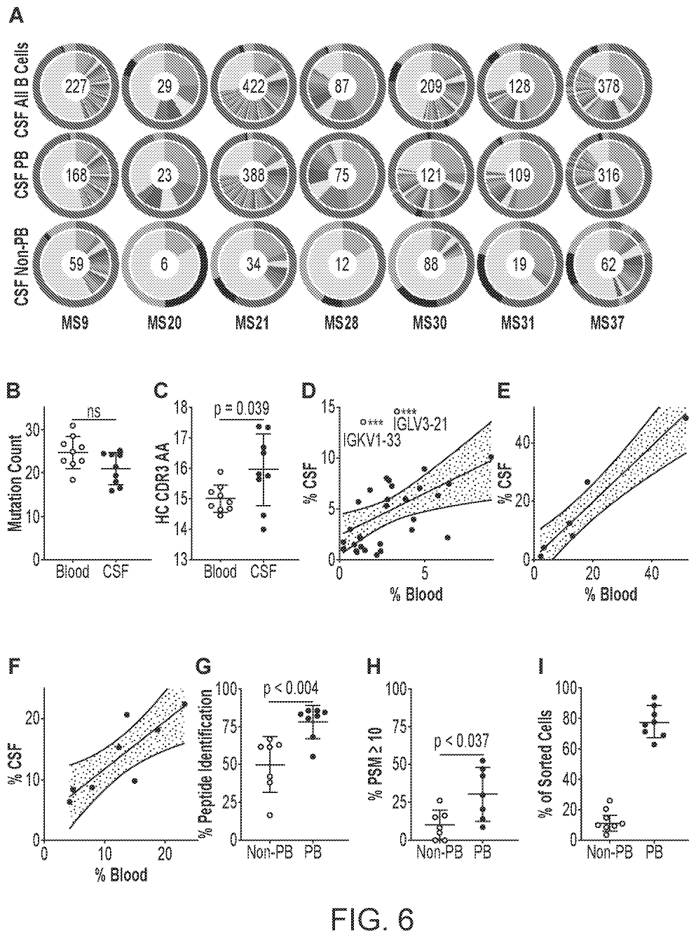

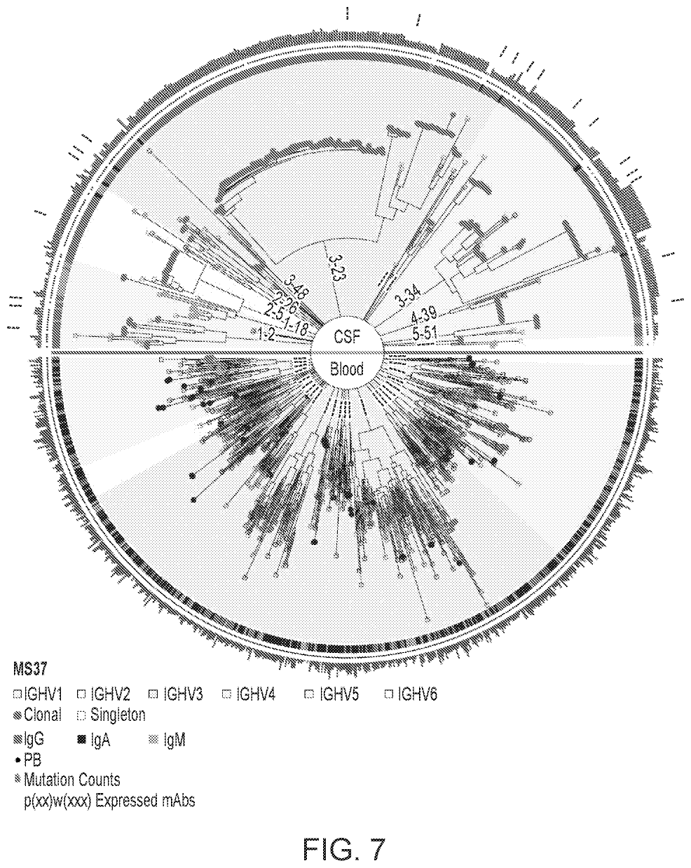

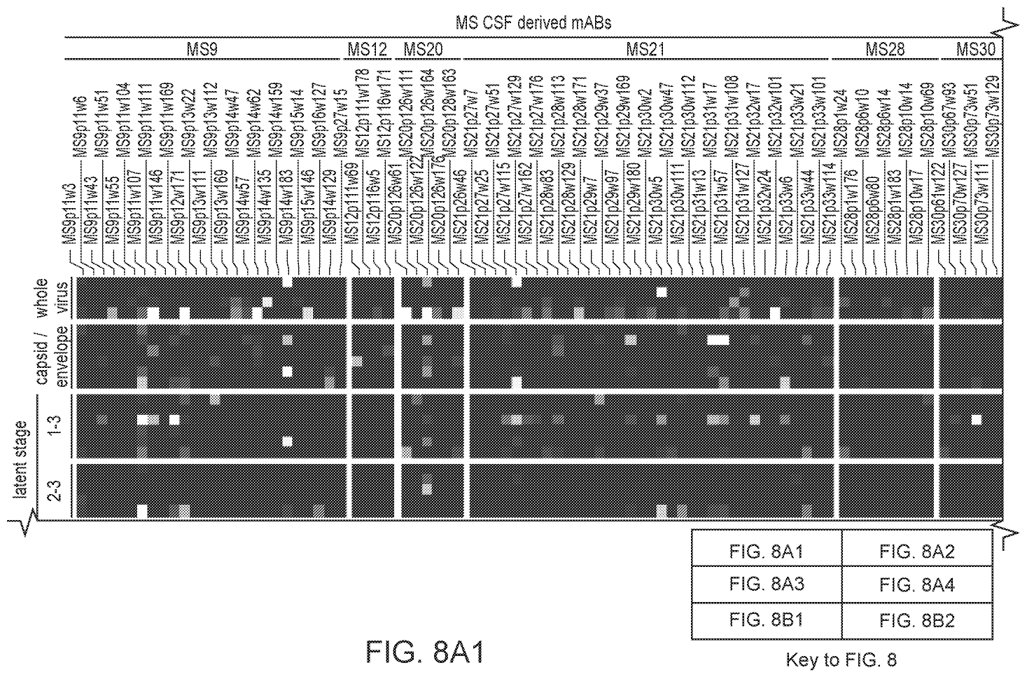

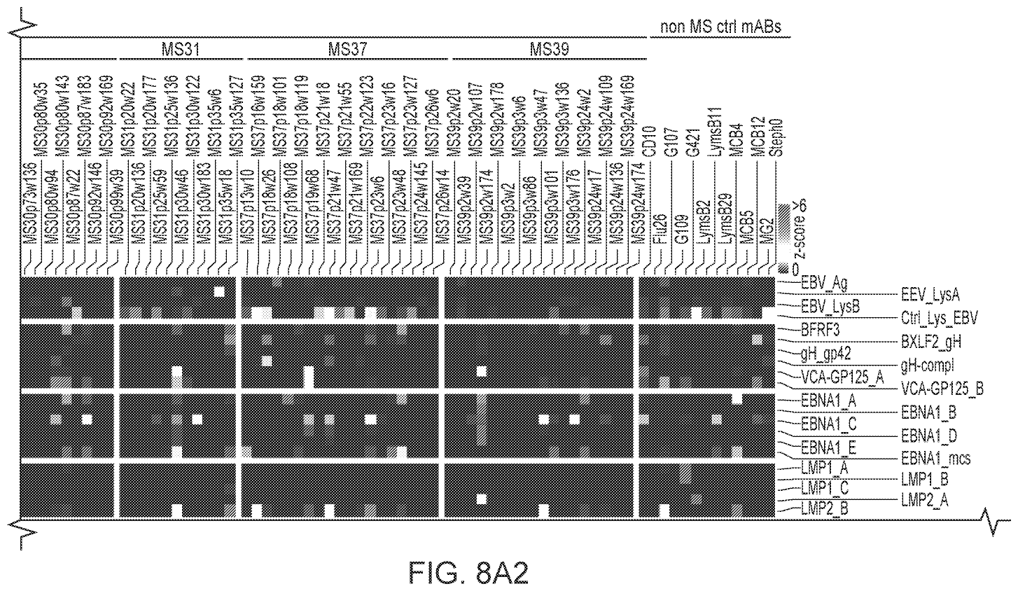

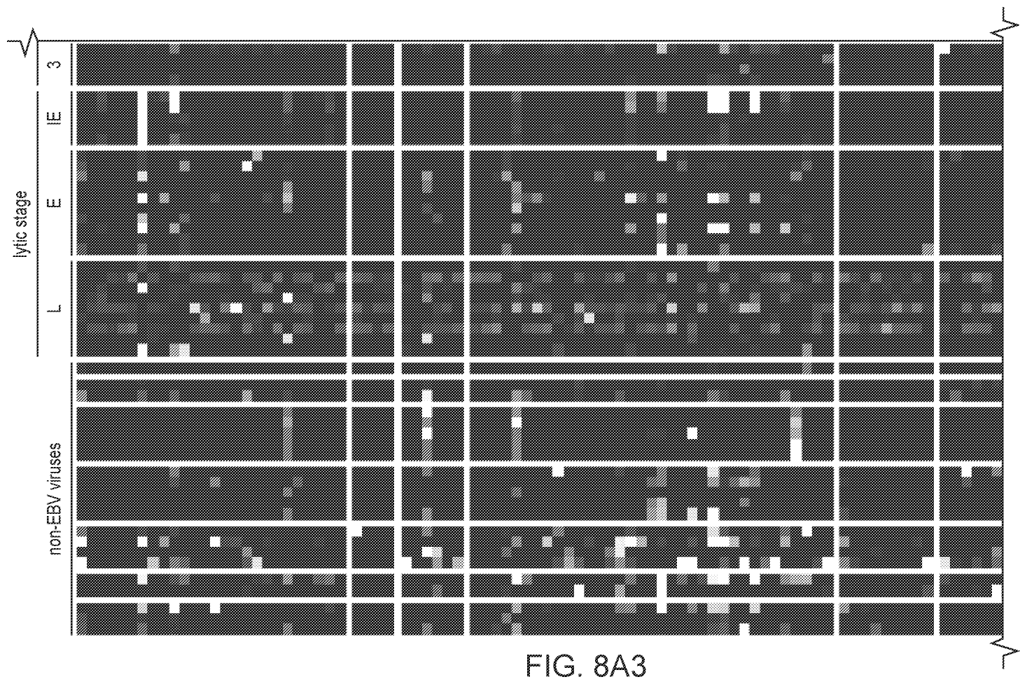

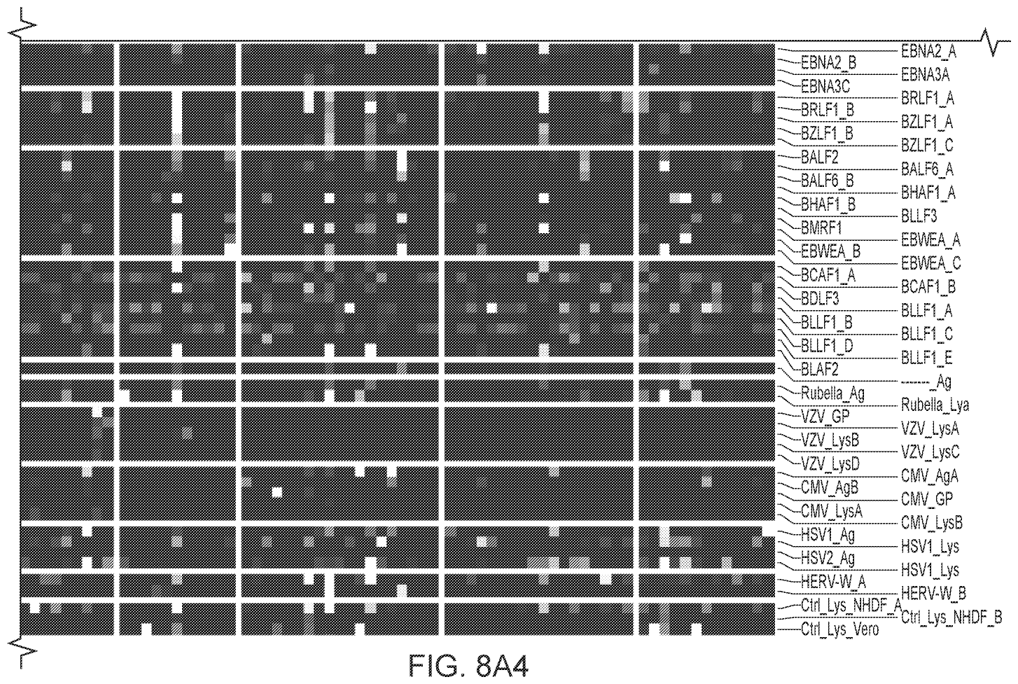

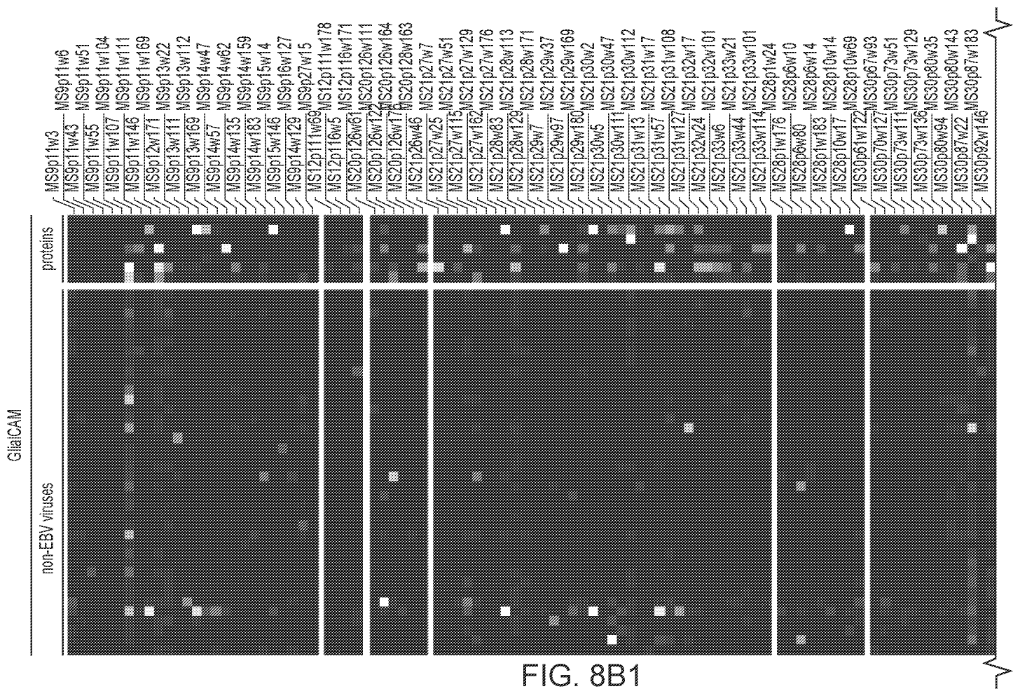

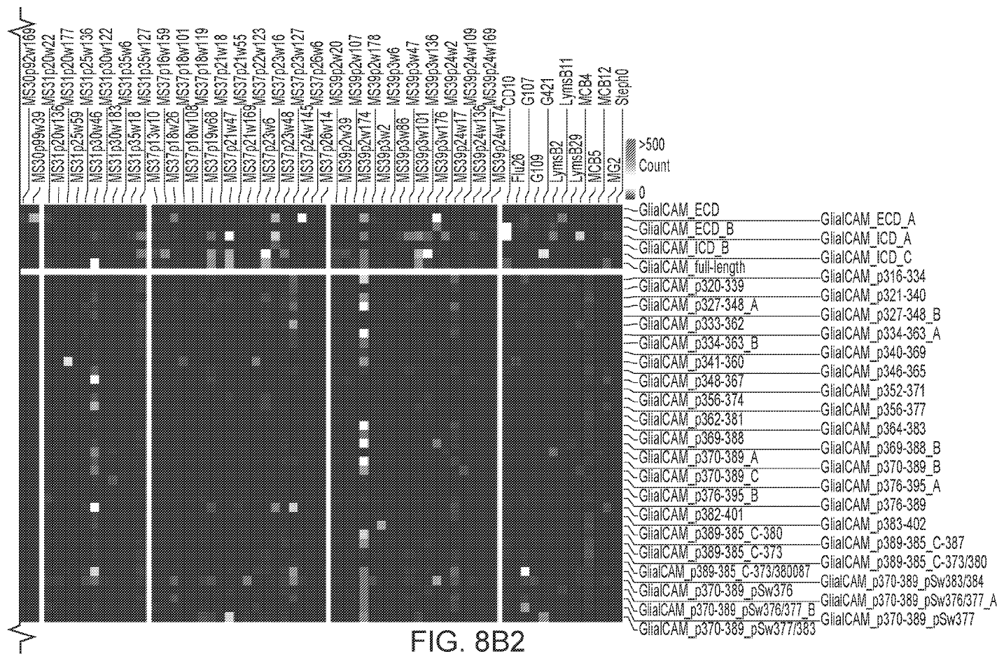

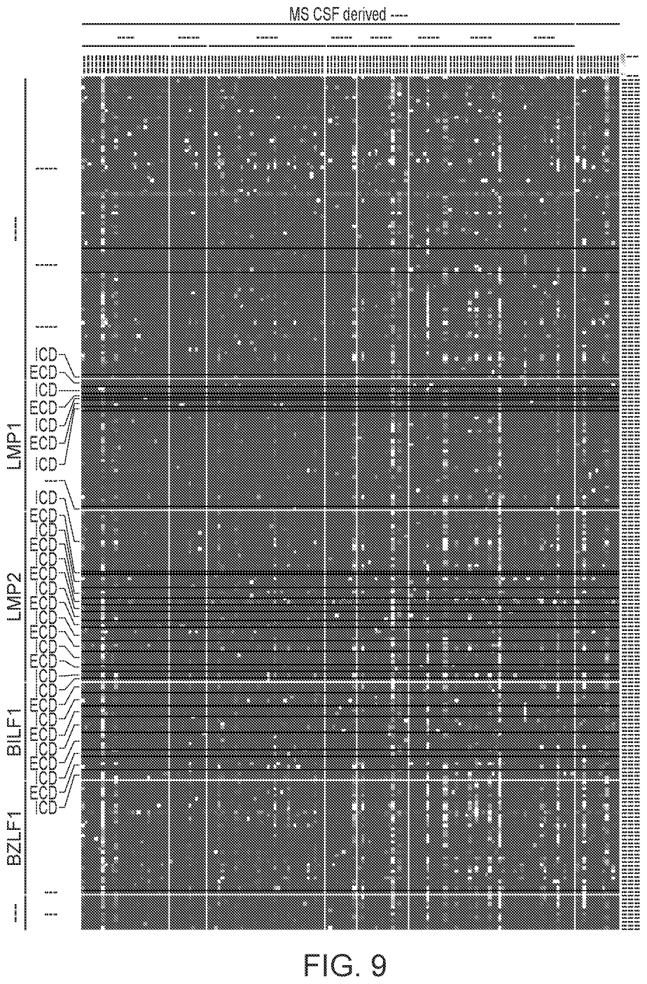

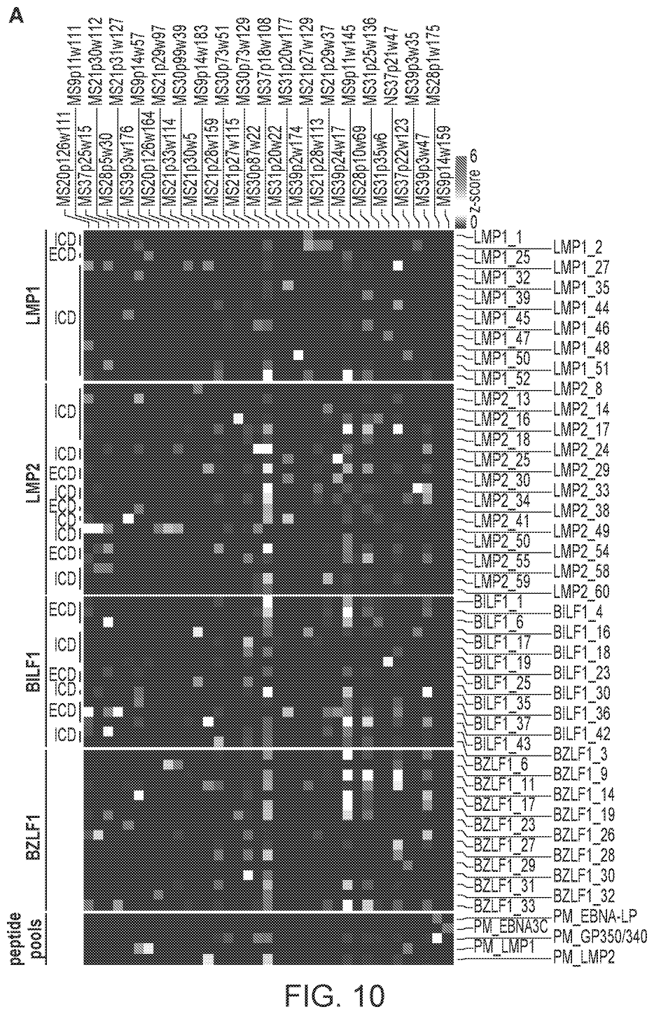

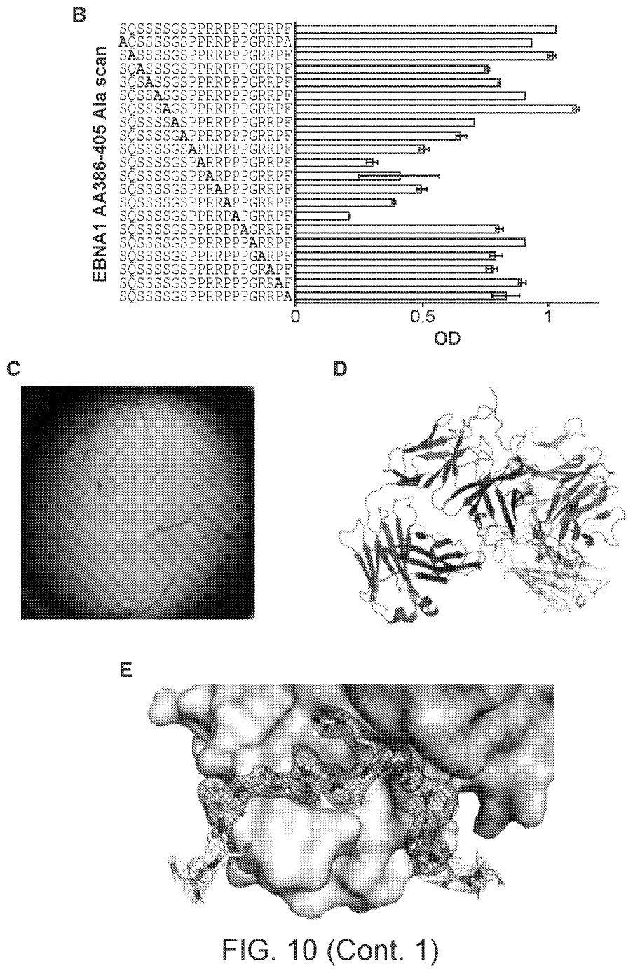

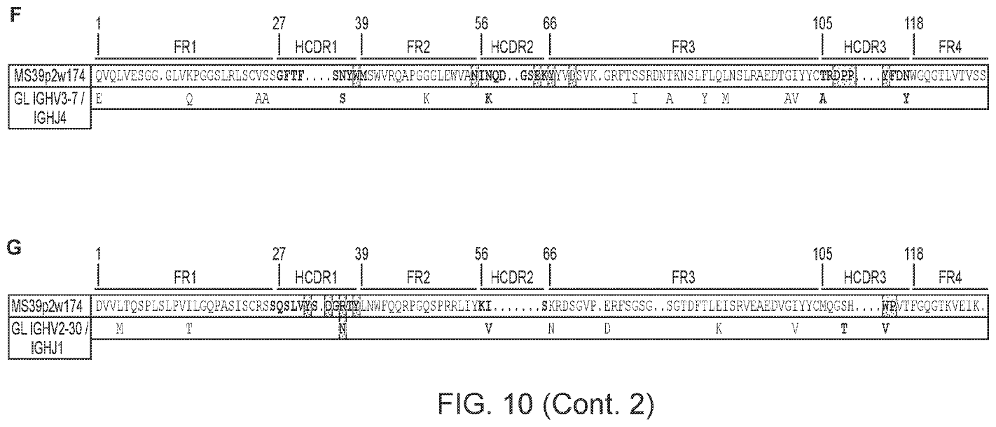

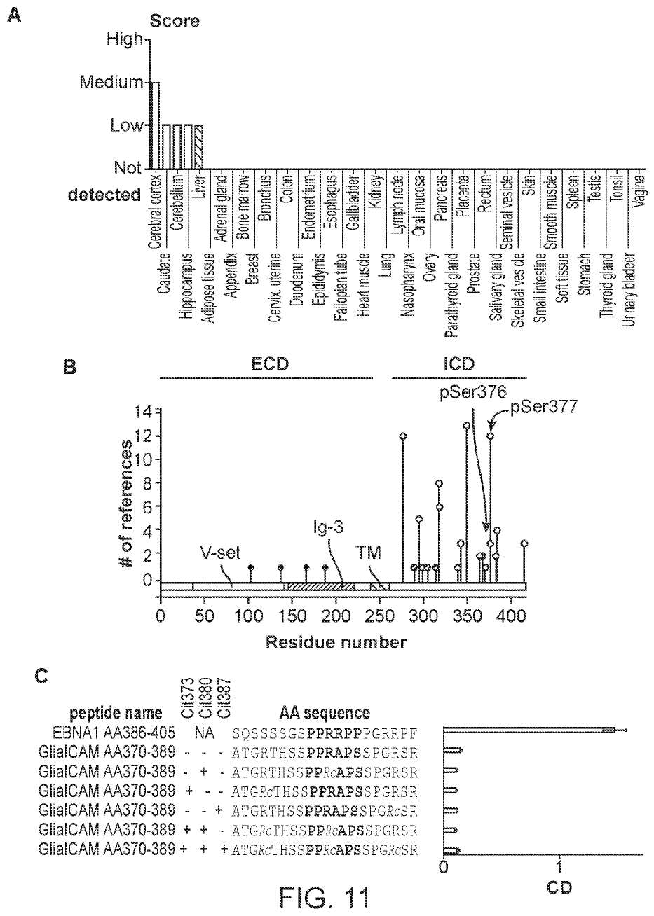

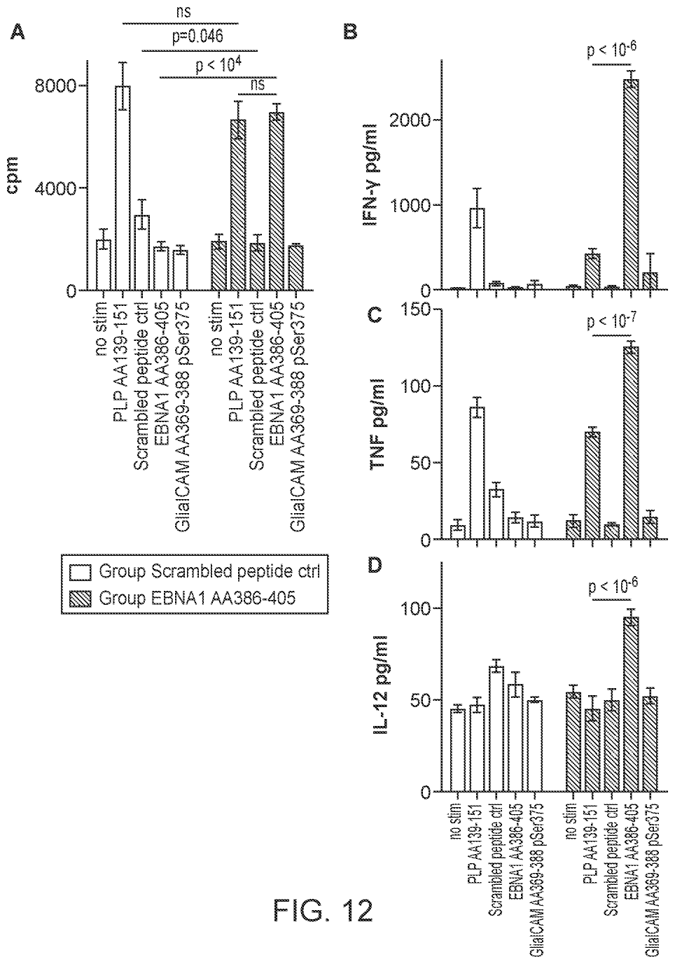

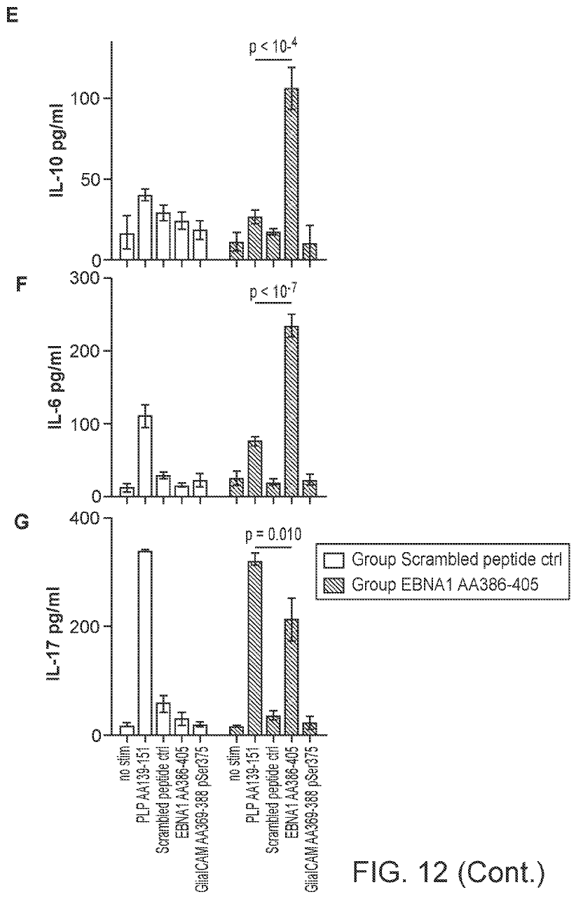

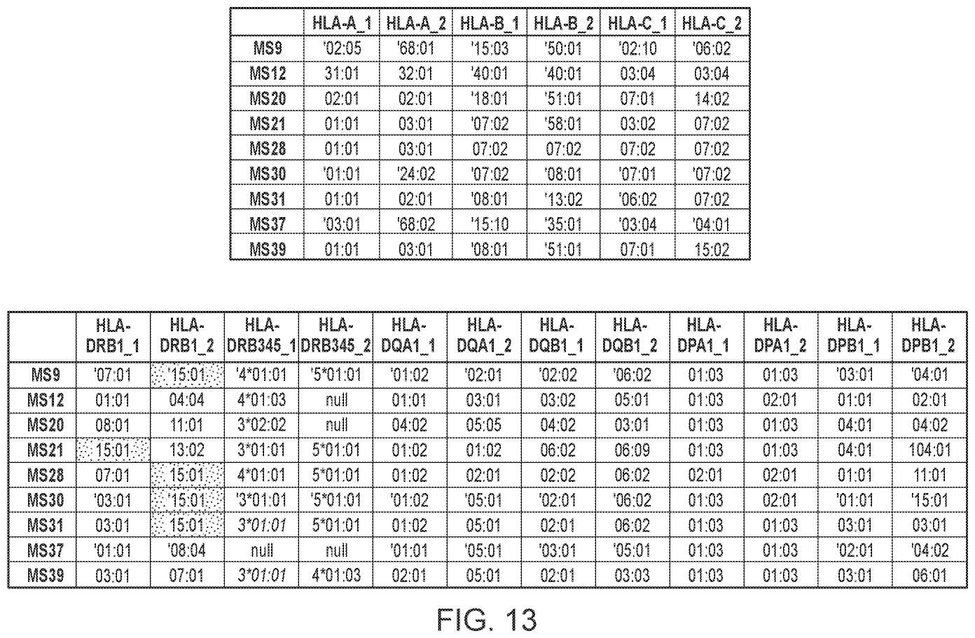

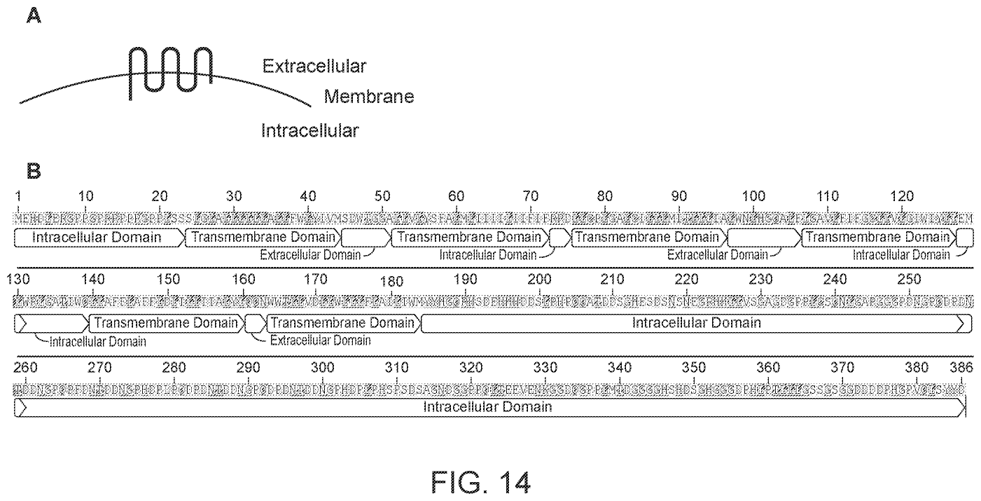

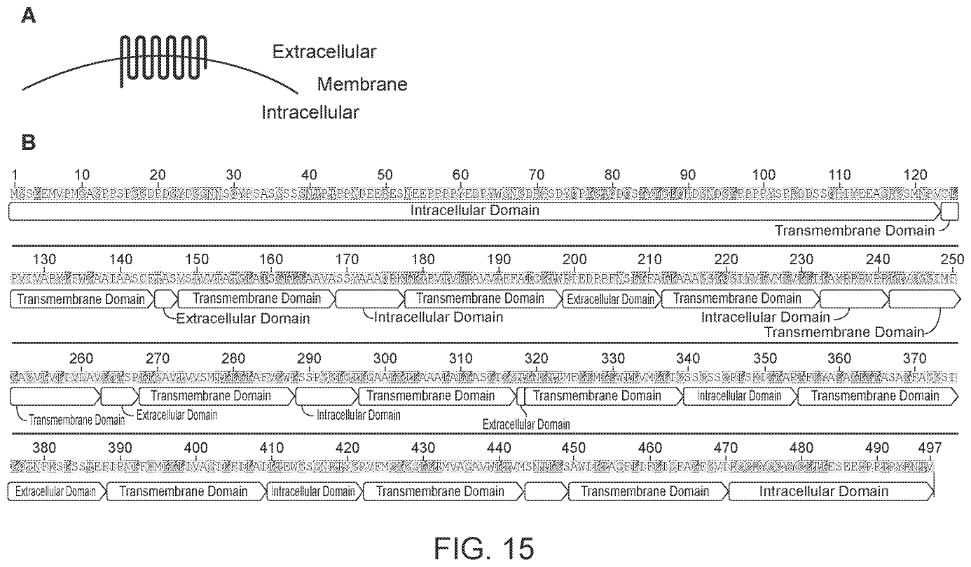

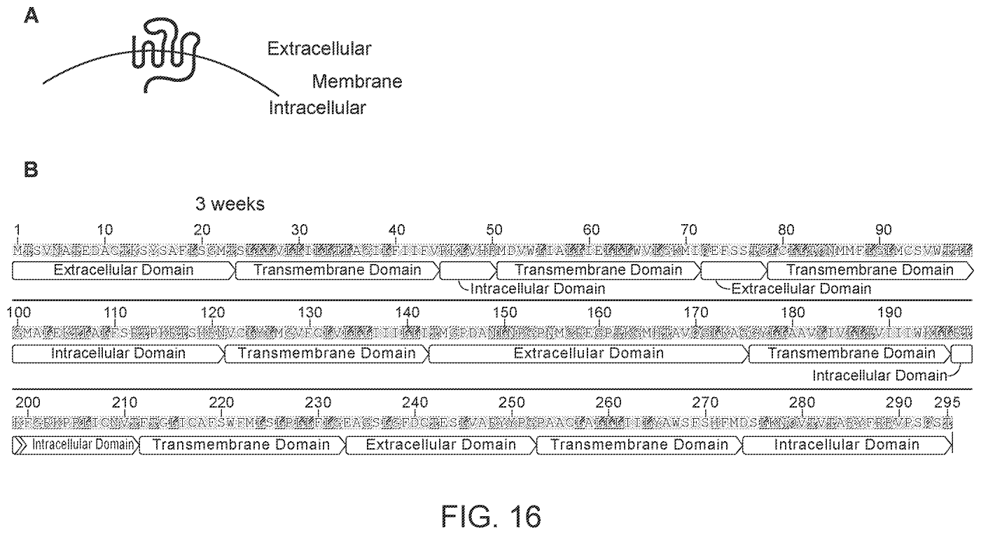

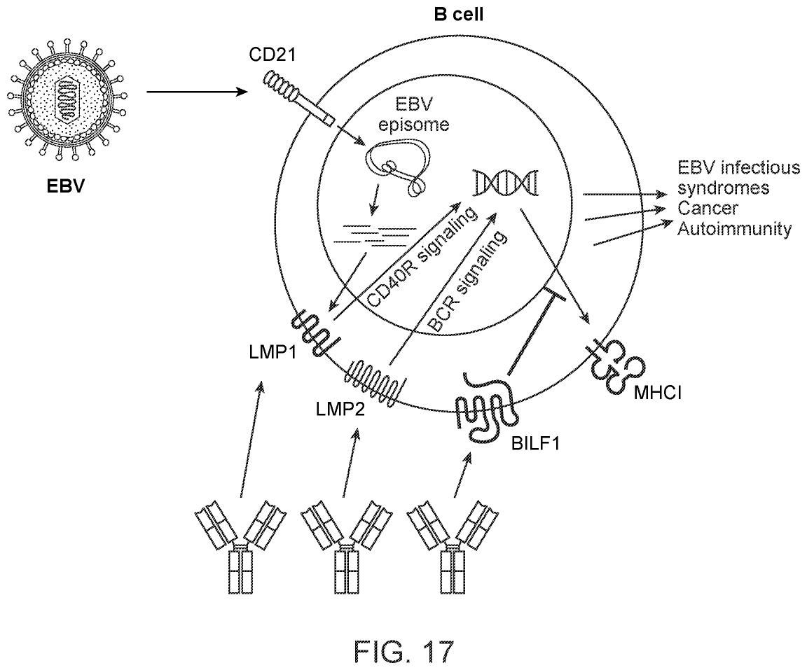

We proceeded with testing for cross-reactive antibodies to EBNA1 AA386-405 and GlialCAM could be detected in plasma of MS patients. we tested immunoglobulin reactivities in plasma from a cohort of 36 MS patients and 20 healthy controls. >99.9% of MS patients have been infected with EBV. As expected, all MS patients show elevated titers against EBNA1 protein, whereas 3 of 20 healthy individuals showed no IgG titer indicating previous EBV infection. Specific reactivity against EBNA1 AA386-405 was observed in 8/36 of MS patients (22.2% vs. 0% in control group, threshold set at mean+4 SD of control group) (FIG. 41). 3 of the 6 samples highest reactive to EBNA1 AA386-405 show also the highest reactivity to GlialCAM ICD (FIG. 4J). 5 of 36 MS patients show high reactivity against GlialCAM ICD (13.9% vs. 0% in control group, threshold set at mean+4 SD of control group).

22 of the selected 148 mABs, isolated from 8 of 9 patients, showed reactivity to GlialCAM, either to the intracellular domain, or to the extracellular domain (FIG. 2J). No additional mAB was identified that bound to same phosphorylated epitope AA370-389, but two mABs bound the non-the same peptide in its non-phosphorylated form. Both mABs also showed cross-reactivity to the Gly-Ala-rich low-complexity region of EBNA1 (FIG. 2A,B,J, blue highlighted mABs).

Of note, a similar proline-rich region on myelin basic protein (MBP) has been described extensively, but we could not show any binding of MS39p2w174 to MBP protein, which does not exclude the possibility that other antibodies against EBNA1 AA386-405 might cross-react to the proline-rich region on MBP.

Immunization with EBNA1 AA386-405 generates anti-GlialCAM antibodies in mice and aggravates experimental autoimmune encephalomyelitis (EAE). To assess the effect of antibodies against EBNA1 AA386-405 in-vivo, we used the mouse model EAE. SJL mice were immunized with EBNA1 AA386-405 or scrambled control peptide (SPSRPGRSRSRGSPFPQPSP, not binding to MS39p2w174, see FIG. 2I). EAE was induced three weeks after the initial immunization with a second immunization of the same respective peptides mixed with PLP AA135-151. Mice in the EBNA1 group generated a robust antibody response to both EBNA1 AA386-405 (FIG. 4A) as well as GlialCAM protein and phospho-GlialCAM p7 (FIG. 4A-C). The EBNA1 group showed more severe symptoms of paresis, in particular during the initial peak of disease, and subsequent relapses occurred earlier and were more severe (FIG. 4D), which was most pronounced in the mice with the highest titers of anti-GlialCAM antibodies (FIG. 4F) EBNA1 promoted the infiltration of T cells and Mac3-positive myeloid cells into the CNS and enhanced demyelination (FIG. 4G,H). In addition to the B cell response, EBNA1 AA386-405 induced a strong antigen-specific T cell response, comparable to PLP AA135-151. The PLP AA135-151 specific T cell response was comparable in both groups. Fitting to the strong antibody response, EBNA1 AA386-405 specific T helper cells (Th) produced more Th1 cytokines (IFN-γ, TNF, IL-12,) and less IL-17. No robust Th cell response against GlialCAM AA369-388 pSer375 could be detected.

### REFERENCES

1. K. Hawker, P. O'Connor, M. S. Freedman, P. A. Calabresi, J. Antel, J. Simon, S. L. Hauser, E. Waubant, T. Vollmer, H. Panitch, J. Zhang, P. Chin, C. H. Smith, O. trial group, Rituximab in patients with primary progressive multiple sclerosis: results of a randomized double-blind placebo-controlled multicenter trial. Ann. Neurol. 66, 460-471 (2009).
2. L. Kappos, D. Li, P. A. Calabresi, P. O'Connor, A. Bar-Or, F. Barkhof, M. Yin, D. Leppert, R. Glanzman, J. Tinbergen, S. L. Hauser, Ocrelizumab in relapsing-remitting multiple sclerosis: a phase 2, randomised, placebo-controlled, multicentre trial. Lancet. 378, 1779-1787 (2011).
3. X. Montalban, S. L. Hauser, L. Kappos, D. L. Arnold, A. Bar-Or, G. Comi, J. de Seze, G. Giovannoni, H.-P. Hartung, B. Hemmer, F. Lublin, K. W. Rammohan, K. Selmaj, A. Traboulsee, A. Sauter, D. Masterman, P. Fontoura, S. Belachew, H. Garren, N. Mairon, P. Chin, J. S. Wolinsky, O. C. Investigators, Ocrelizumab versus Placebo in Primary Progressive Multiple Sclerosis. N. Engl. J. Med. 376, 209-220 (2017).
4. A. Negron, R. R. Robinson, O. Stuve, T. G. Forsthuber, The role of B cells in multiple sclerosis: Current and future therapies. Cell. Immunol. 339, 10-23 (2019).
5. T. V. Lanz, A.-K. Probstel, I. Mildenberger, M. Platten, L. Schirmer, Single-Cell High-Throughput Technologies in Cerebrospinal Fluid Research and Diagnostics. Front. Immunol. 10, 1302 (2019).
6. D. Schafflick, C. A. Xu, M. Hartlehnert, M. Cole, T. Lautwein, A. Schulte-Mecklenbeck, J. Wolbert, M. Heming, S. G. Meuth, T. Kuhlmann, C. C. Gross, H. Wiendl, N. Yosef, G. M. zu Horste, Integrated single cell analysis of blood and cerebrospinal fluid leukocytes in multiple sclerosis. 192, 2551.
7. A. Wang, O. Rojas, D. Lee, J. L. Gommerman, Regulation of neuroinflammation by B cells and plasma cells. Immunol. Rev., 1-16 (2020).
8. R. Hohlfeld, K. Dornmair, E. Meinl, H. Wekerle, The search for the target antigens of multiple sclerosis, part 2: CD8+ T cells, B cells, and antibodies in the focus of reverse-translational research. Lancet Neurol. (2016), doi:10.1016/S1474-4422(15)00313-0.
9. E. L. Thacker, F. Mirzaei, A. Ascherio, Infectious mononucleosis and risk for multiple sclerosis: A meta-analysis. Ann. Neurol. 59, 499-503 (2006).
10. A. Ascherio, K. L. Munger, Environmental risk factors for multiple sclerosis. Part I: the role of infection. Ann. Neurol. 61, 288-299 (2007).
11. S. Abrahamyan, B. Eberspächer, M.-M. Hoshi, L. Aly, F. Luessi, S. Groppa, L. Klotz, S. G. Meuth, C. Schroeder, T. Grüter, B. Tackenberg, F. Paul, F. Then-Bergh, T. Kümpfel, F. Weber, M. Stangel, A. Bayas, B. T. Wildemann, C. Heesen, U. Zettl, C. Warnke, G. Antony, N. Hessler, H. Wiendl, S. Bittner, B. Hemmer, R. Gold, A. Salmen, K. Ruprecht, J. Neurol. Neurosurg. Psychiatry, in press (available at http://jnnp.bmj.com/lookup/doi/10.1136/jnnp-2020-322941).
12. A. K. Hedström, J. Huang, A. Michel, J. Butt, N. Brenner, J. Hillert, T. Waterboer, I. Kockum, T. Olsson, L. Alfredsson, High Levels of Epstein-Barr Virus Nuclear Antigen-1-Specific Antibodies and Infectious Mononucleosis Act Both Independently and Synergistically to Increase Multiple Sclerosis Risk. Front. Neurol. 10, 1368 (2019).
13. K. Ruprecht, B. Wunderlich, R. Gieß, P. Meyer, M. Loebel, K. Lenz, J. Hofmann, B. Rosche, O. Wengert, F. Paul, U. Reimer, C. Scheibenbogen, Multiple sclerosis: The elevated antibody response to Epstein-Barr virus primarily targets, but is not confined to, the glycine-alanine repeat of Epstein-Barr nuclear antigen-1. J. Neuroimmunol. 272, 56-61 (2014).
14. N. Jafari, G. P. van Nierop, G. M. G. M. Verjans, A. D. M. E. Osterhaus, J. M. Middeldorp, R. Q. Hintzen, No evidence for intrathecal IgG synthesis to Epstein Barr virus nuclear antigen-1 in multiple sclerosis. J. Clin. Virol. 49, 26-31 (2010).
15. J. Salzer, M. Nyström, G. Hallmans, H. Stenlund, G. Wadell, P. Sundström, Epstein-Barr virus antibodies and vitamin D in prospective multiple sclerosis biobank samples. Mult. Scler. 19, 1587-1591 (2013).
16. K. Tengvall, J. Huang, C. Hellström, P. Kammer, M. Biström, B. Ayoglu, I. Lima Bomfim, P. Stridh, J. Butt, N. Brenner, A. Michel, K. Lundberg, L. Padyukov, I. E. Lundberg, E. Svenungsson, I. Ernberg, S. Olafsson, A. T. Dilthey, J. Hillert, L. Alfredsson, P. Sundström, P. Nilsson, T. Waterboer, T. Olsson, I. Kockum, Molecular mimicry between Anoctamin 2 and Epstein-Barr virus nuclear antigen 1 associates with multiple sclerosis risk. Proc. Natl. Acad. Sci. U.S.A. 5, 201902623-201916960 (2019).
17. Z. Wang, P. G. Kennedy, C. Dupree, M. Wang, C. Lee, T. Pointon, T. D. Langford, M. W. Graner, X. Yu, Antibodies from Multiple Sclerosis Brain Identified Epstein-Barr Virus Nuclear Antigen 1 & 2 Epitopes which Are Recognized by Oligoclonal Bands. J. Neuroimmune Pharmacol. (2020), doi:10.1007/s11481-020-09948-1.
18. A. Gilbert, X. E. Vidal, R. Estevez, M. Cohen-Salmon, A.-C. Boulay, Postnatal development of the astrocyte perivascular MLC1/GlialCAM complex defines a temporal window for the gliovascular unit maturation. Brain Struct. Funct. 7, 12-41 (2019).
19. L. Favre-Kontula, A. Rolland, L. Bernasconi, M. Karmirantzou, C. Power, B. Antonsson, U. Boschert, GlialCAM, an immunoglobulin-like cell adhesion molecule is expressed in glial cells of the central nervous system. Glia. 56, 633-645 (2008).
20. T. López-Hernandez, M. C. Ridder, M. Montolio, X. Capdevila-Nortes, E. Polder, S. Sirisi, A. Duarri, U. Schulte, B. Fakler, V. Nunes, G. C. Scheper, A. Martínez, R. Estevez, M. S. van der Knaap, Mutant GlialCAM causes megalencephalic leukoencephalopathy with subcortical cysts, benign familial macrocephaly, and macrocephaly with retardation and autism. Am. J. Hum. Genet. 88, 422-432 (2011).
21. M. Bugiani, M. Dubey, M. Breur, N. L. Postma, M. P. Dekker, T. Ter Braak, U. Boschert, T. E. M. Abbink, H. D. Mansvelder, R. Min, J. R. T. van Weering, M. S. van der Knaap, Megalencephalic leukoencephalopathy with cysts: the Glialcam-null mouse model. Ann. Clin. Transl. Neurol. 4, 450-465 (2017).
22. T. López-Hernández, S. Sirisi, X. Capdevila-Nortes, M. Montolio, V. Fernández-Dueñas, G. C. Scheper, M. S. van der Knaap, P. Casquero, F. Ciruela, I. Ferrer, V. Nunes, R. Estevez, Molecular mechanisms of MLC1 and GLIALCAM mutations in megalencephalic leukoencephalopathy with subcortical cysts. Hum. Mol. Genet. 20, 3266-3277 (2011).
23. E. Jeworutzki, T. López-Hernández, X. Capdevila-Nortes, S. Sirisi, L. Bengtsson, M. Montolio, G. Zifarelli, T. Arnedo, C. S. Müller, U. Schulte, V. Nunes, A. Martínez, T. J. Jentsch, X. Gasull, M. Pusch, R. Estevez, GlialCAM, a Protein Defective in a Leukodystrophy, Serves as a CIC-2 CI-Channel Auxiliary Subunit. Neuron. 73, 951-961 (2012).

### Example 2

### Detection of EBV-Infected B Cells to Identify Autoimmune Disease Patients Likely to Respond to EBV-Infected B Cell Depleting Therapeutics, and to Monitor Response to EBV-Infected B Cell Depleting Therapeutics

Patients with MS, T1D, SS, RA, DM or another EBV-associated autoimmune disease are tested for EBV-infected by cells in their blood, spinal fluid or other biological sample. EBV-infected B cells are detected by PCR to detect EBV genes, or by immunostaining to detect B cell expression of EBV proteins, or by flow cytometry to detect B cell expression of EBV proteins and/or genes. Autoimmune patients exhibiting EBV-infected B cell are then treated with EBV-infected B cell depleting therapeutics that include monoclonal antibodies targeting one or more of the following EBV protein expressed on the surface of EBV-infected B cells: LMP1; LMP2; BILF1; LMP1+BILF1; LMP2+BILF1; LMP1+LMP2; or LMP1+LMP2+BILF1. 1-4 months following EBV-infected B cell depleting monoclonal antibody treatment, patients exhibit improvement in their corresponding autoimmune disease activity scores for MS, T1D, SS, RA, DM or other autoimmune disease. Response to EBV-infected B cell depleting therapeutic monoclonal antibodies can be monitored by post-treatment PCR, immunostaining, and/or flow cytometry; and reduction of EBV-infected B cells is associated with clinical improvement. Autoimmune patients can be monitored with EBV-infected B cell detection by PCR, immunostaining and/or flow cytometry to determine when additional treatment courses with EBV-infected B cell depleting monoclonal antibody therapy is indicated for effective disease control.

### Example 3

Depletion of EBV-Infected B cells to Treat EBV-Associated Autoimmune Disease

Patients with MS, T1D, SS, RA, DM or another EBV-associated autoimmune disease are treated with EBV-infected B cell depleting therapeutics that include monoclonal antibodies targeting one or more of the following EBV protein expressed on the surface of EBV-infected B cells: LMP1; LMP2; BILF1; LMP1+BILF1; LMP2+BILF1; LMP1+LMP2; or LMP1+LMP2+BILF1. 1-4 months following EBV-infected B cell depleting monoclonal antibody treatment, patients exhibit improvement in their corresponding autoimmune disease activity scores for MS, T1D, SS, RA, DM or other autoimmune disease.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

TABLE 1
Detailed descriptions and sources of peptide antigens on EBV antigen arrays

| Antigen | | Antigen | | Antigen | |
|---|---|---|---|---|---|
| Name | AA Sequence | Name | AA Sequence | Name | AA Sequence |
| EBNA1_1 | MSDEGPGTGPGNGLGEKGDT | EBNA1_81 | YFMVFLQTHIFAEVLKDAIK | LMP2_4 | GYDGGNNSQYPSASGSSGNT |
| EBNA1_2 | TGPGNGLGEKGDTSGPEGSG | EBNA1_82 | THIFAEVLKDAIKDLVMTKP | LMP2_5 | SQYPSASGSSGNTPTPPNDE |
| EBNA1_3 | GEKGDTSGPEGSGGSGPQRR | EBNA1_83 | LKDAIKDLVMTKPAPTCNIR | LMP2_6 | GSSGNTPTPPNDEERESNEE |
| EBNA1_4 | GPEGSGGSGPQRRGGDNHGR | EBNA1_84 | LVMTKPAPTCNIRVTVCSFD | LMP2_7 | TPPNDEERESNEEPPPPYED |
| EBNA1_5 | SGPQRRGGDNHGRGRGRGRG | EBNA1_85 | PTCNIRVTVCSFDDGVDLPP | LMP2_8 | RESNEEPPPPYEDPYWGNGD |
| EBNA1_6 | GDNHGRGRGRGRGRGGGRPG | EBNA1_86 | TVCSFDDGVDLPPWFPPMVE | LMP2_9 | PPPYEDPYWGNGDRHSDYQP |
| EBNA1_7 | RGRGRGRGGGRPGAPGGSGS | EBNA1_87 | GVDLPPWFPPMVEGAAAEGD | LMP2_10 | YWGNGDRHSDYQPLGTQDQS |
| EBNA1_8 | GGGRPGAPGGSGSGPRHRDG | EBNA1_88 | FPPMVEGAAAEGDDGDDGDE | LMP2_11 | HSDYQPLGTQDQSLYLGLQH |
| EBNA1_9 | PGGSGSGPRHRDGVRRPQKR | EBNA1_89 | AAAEGDDGDDGDEGGDGDEG | LMP2_12 | GTQDQSLYLGLQHDGNDGLP |
| EBNA1_10 | PRHRDGVRRPQKRPSCIGCK | EBNA1_90 | GDDGDEGGDGDEGEEGQE | LMP2_13 | YLGLQHDGNDGLPPPPYSPR |
| EBNA1_11 | RRPQKRPSCIGCKGTHGGTG | BILF1_1 | LSTMAPGSTVGTLVANMTSV | LMP2_14 | GNDGLPPPPYSPRDDSSQHI |
| EBNA1_12 | SCIGCKGTHGGTGAGAGAGG | BILF1_4 | ATEDACTKSYSAFLSGMTSL | LMP2_15 | PPYSPRDDSSQHIYEEAGRG |
| EBNA1_13 | THGGTGAGAGAGGAGAGGAG | BILF1_6 | SGMTSLLLVLLILLTLAGIL | LMP2_16 | DSSQHIYEEAGRGSMNPVCL |
| EBNA1_14 | GAGAGGAGAGGAGAGGGAGA | BILF1_7 | LVLLILLTLAGILFIIFVRK | LMP2_17 | EEAGRGSMNPVCLPVIVAPY |
| EBNA1_15 | GAGGAGAGGGAGAGGGAGGA | BILF1_8 | TLAGILFIIFVRKLVHRMDV | LMP2_18 | MNPVCLPVIVAPYLFWLAAI |
| EBNA1_16 | GGGAGAGGGAGGAGGAGGAG | BILF1_9 | IIFVRKLVHRMDVWLIALLI | LMP2_19 | VIVAPYLFWLAAIAASCFTA |
| EBNA1_17 | GGAGGAGGAGGAGAGGGAGA | BILF1_10 | VHRMDVWLIALLIELLLWVL | LMP2_23 | TGLALSLLLLAAVASSYAAA |
| EBNA1_18 | GAGGAGAGGGAGAGGGAGGA | BILF1_12 | LLLWVLGKMIQEFSSTGLCL | LMP2_24 | LLLAAVASSYAAAQRKLLTP |
| EBNA1_19 | GGGAGAGGGAGGAGGAGAGG | BILF1_13 | KMIQEFSSTGLCLLTQNMMF | LMP2_25 | SSYAAAQRKLLTPVTVLTAV |
| EBNA1_20 | GGAGGAGGAGAGGGAGAGGG | BILF1_14 | STGLCLLTQNMMFLGLMCSV | LMP2_26 | RKLLTPVTVLTAVVTFFAIC |
| EBNA1_21 | GAGAGGGAGAGGGAGGAGAG | BILF1_15 | TQNMMFLGLMCSVWTHLGMA | LMP2_28 | TFFAICLTWRIEDPPFNSLL |
| EBNA1_22 | AGAGGGAGGAGAGGGAGGAG | BILF1_16 | GLMCSVWTHLGMALEKTLAL | LMP2_29 | TWRIEDPPFNSLLFALLAAA |
| EBNA1_23 | GGAGAGGGAGGAGGAGAGGG | BILF1_17 | THLGMALEKTLALFSRTPKR | LMP2_30 | PFNSLLFALLAAAGGLQGIY |
| EBNA1_24 | GAGGAGGAGAGGGAGAGGGA | BILF1_18 | EKTLALFSRTPKRTSHRNVC | LMP2_33 | LVMLVLLILAYRRRWRRLTV |
| EBNA1_25 | AGAGGGAGAGGGAGGAGAGG | BILF1_19 | SRTPKRTSHRNVCLYLMGVF | LMP2_34 | ILAYRRRWRRLTVCGGIMFL |
| EBNA1_26 | GAGGGAGGAGAGGGAGGAGG | BILF1_23 | ILLITMGPDANLNRGPNMCR | LMP2_38 | DAVLQLSPLLGAVTVVSMTL |
| EBNA1_27 | GAGAGGGAGGAGGAGAGGGA | BILF1_24 | PDANLNRGPNMCREGPTKGM | LMP2_39 | PLLGAVTVVSMTLLLLAFVL |
| EBNA1_28 | AGGAGGAGAGGGAGAGGAGG | BILF1_25 | GPNMCREGPTKGMHTAVQGL | LMP2_40 | VVSMTLLLLAFVLWLSSPGG |
| EBNA1_29 | GAGGGAGAGGAGGAGGAGAG | BILF1_26 | GPTKGMHTAVQGLKAGCYLL | LMP2_41 | LLAFVLWLSSPGGLGTLGAA |
| EBNA1_30 | AGGAGGAGGAGAGGAGAGGG | BILF1_30 | TVIIIWKLLRTKFGRKPRLI | LMP2_42 | LSSPGGLGTLGAALLTLAAA |
| EBNA1_31 | GGAGAGGAGAGGGAGGAGGA | BILF1_31 | LLRTKFGRKPRLICNVTFTG | LMP2_44 | LTLAAALALLASLILGTLNL |
| EBNA1_32 | AGAGGGAGGAGGAGAGGAGA | BILF1_32 | RKPRLICNVTFTGLICAFSW | LMP2_46 | LGTLNLTTMFLLMLLWTLVV |
| EBNA1_33 | GGAGGAGAGGAGAGGAGAGG | BILF1_34 | ICAFSWFMLSLPLLFLGEAG | LMP2_48 | LWTLVVLLICSSCSSCPLSK |
| EBNA1_34 | AGGAGAGGAGAGGAGAGGAG | BILF1_35 | MLSLPLLFLGEAGSLGFDCT | LMP2_49 | LICSSCSSCPLSKILLARLF |
| EBNA1_35 | GAGAGGAGAGGAGGAGAGGA | BILF1_36 | FLGEAGSLGFDCTESLVARY | LMP2_50 | SCPLSKILLARLFLYALALL |
| EBNA1_36 | GAGGAGGAGAGGAGGAGAGG | BILF1_37 | LGFDCTESLVARYYPGPAAC | LMP2_51 | LLARLFLYALALLLLASALI |
| EBNA1_37 | AGAGGAGGAGAGGAGGAGAG | BILF1_41 | YAWSFSHFMDSLKNQVTVTA | LMP2_53 | LASALIAGGSILQTNFKSLS |
| EBNA1_38 | GAGAGGAGGAGAGGGAGGAG | BILF1_42 | FMDSLKNQVTVTARYFRRVP | LMP2_54 | GGSILQTNFKSLSSTEFIPN |
| EBNA1_39 | GGAGAGGGAGGAGAGGGAGG | BILF1_43 | QVTVTARYFRRVPSQST | LMP2_55 | NFKSLSSTEFIPNLFCMLLL |
| EBNA1_40 | GAGGAGAGGGAGGAGAGGAG | LMP1_1 | MEHDLERGPPGPRRPPRGPP | LMP2_58 | VAGILFILAILTEWGSGNRT |
| EBNA1_41 | GGGAGGAGAGGAGGAGAGGA | LMP1_2 | GPPGPRRPPRGPPLSSSLGL | LMP2_59 | LAILTEWGSGNRTYGPVFMC |
| EBNA1_42 | GAGGAGGAGAGGAGGAGAGG | LMP1_6 | LLFWLYIVMSDWTGGALLVL | LMP2_60 | GSGNRTYGPVFMCLGGLLTM |
| EBNA1_43 | AGAGGAGGAGAGGAGGAGAG | LMP1_7 | VMSDWTGGALLVLYSFALML | LMP2_61 | GPVFMCLGGLLTMVAGAVWL |
| EBNA1_44 | GAGAGGAGGAGAGGGAGAGG | LMP1_11 | IFRRDLLCPLGALCILLLMI | LMP2_62 | GGLLTMVAGAVWLTVMSNTL |
| EBNA1_45 | GGAGAGGGAGAGGAGAGGGG | LMP1_13 | ILLLMITLLLIALWNLHGQA | LMP2_63 | AGAVWLTVMSNTLLSAWILT |
| EBNA1_46 | GAGAGGAGAGGGGRGRGGSG | LMP1_18 | LGIWIYLLEMLWRIGATIWQ | LMP2_68 | IRCCRYCCYYCLTLESEERP |
| EBNA1_47 | GAGGGGRGRGGSGGRGRGGS | LMP1_19 | LEMLWRLGATIWQLLAFFLA | LMP2_69 | CYYCLTLESEERPPTPYRNTV |
| EBNA1_48 | GRGGSGGRGRGGSGGRGRGG | LMP1_23 | IIALYLQQNWWTLLVDLLWL | BZLF1_1 | MMDPNSTSEDVKFTPDPYQV |
| EBNA1_49 | RGRGGSGGRGRGGSGGRRGR | LMP1_25 | VDLLWLLLFLAILIWMYYHG | BZLF1_2 | SEDVKFTPDPYQVPFVQAFD |
| EBNA1_50 | GRGRGGSGGRRGRGRERARG | LMP1_26 | LFLAILIWMYYHGQRHSDEH | BZLF1_3 | PDPYQVPFVQAFDQATRVYQ |
| EBNA1_51 | GGRRGRGRERARGGSRERAR | LMP1_27 | WMYYHGQRHSDEHHHDDSLP | BZLF1_4 | FVQAFDQATRVYQDLGGPSQ |
| EBNA1_52 | RERARGGSRERARGRGRGRG | LMP1_28 | RHSDEHHHDDSLPHPQQATD | BZLF1_5 | ATRVYQDLGGPSQAPLPCVL |
| EBNA1_53 | SRERARGRGRGRGEKRPRSP | LMP1_29 | HDDSLPHPQQATDDSGHESD | BZLF1_6 | LGGPSQAPLPCVLWPVLPEP |
| EBNA1_54 | RGRGRGEKRPRSPSSQSSSS | LMP1_30 | PQQATDDSGHESDSNSNEGR | BZLF1_7 | PLPCVLWPVLPEPLPQGQLT |
| EBNA1_55 | KRPRSPSSQSSSSGSPPRRP | LMP1_31 | SGHESDSNSNEGRHHLLVSG | BZLF1_8 | PVLPEPLPQGQLTAYHVSTA |
| EBNA1_56 | SQSSSSGSPPRRPPPGRRPF | LMP1_32 | NSNEGRHHLLVSGAGDGPPL | BZLF1_9 | PQGQLTAYHVSTAPTGSWFS |
| EBNA1_57 | SPPRRPPPGRRPFFHPVGEA | LMP1_33 | HLLVSGAGDGPPLCSQNLGA | BZLF1_10 | YHVSTAPTGSWFSAPQPAPE |
| EBNA1_58 | PGRRPFFHPVGEADYFEYHQ | LMP1_34 | GDGPPLCSQNLGAPGGGPDN | BZLF1_11 | TGSWFSAPQPAPENAYQAYA |
| EBNA1_59 | HPVGEADYFEYHQEGGPDGE | LMP1_35 | SQNLGAPGGGPDNGPQDPDN | BZLF1_12 | PQPAPENAYQAYAAPQLFPV |
| EBNA1_60 | YFEYHQEGGPDGEPDVPPGA | LMP1_36 | GGGPDNGPQDPDNTDDNGPQ | BZLF1_13 | AYQAYAAPQLFPVSDITQNQ |
| EBNA1_61 | GGPDGEPDVPPGAIEQGPAD | LMP1_37 | PQDPDNTDDNGPQDPDNTDD | BZLF1_14 | PQLFPVSDITQNQQTNQAGG |
| EBNA1_62 | DVPPGAIEQGPADDPGEGPS | LMP1_38 | DDNGPQDPDNTDDNGPHDPL | BZLF1_15 | DITQNQQTNQAGGEAPQPGD |
| EBNA1_63 | EQGPADDPGEGPSTGPRGQG | LMP1_39 | PDNTDDNGPHDPLPQDPDNT | BZLF1_16 | TNQAGGEAPQPGDNSTVQTA |
| EBNA1_64 | PGEGPSTGPRGQGDGGRRKK | LMP1_40 | GPHDPLPQDPDNTDDNGPQD | BZLF1_17 | APQPGDNSTVQTAAAVVFAC |
| EBNA1_65 | GPRGQGDGGRRKKGGWFGKH | LMP1_42 | DNGPQDPDNTDDNGPHDPLP | BZLF1_18 | STVQTAAAVVFACPGANQGQ |
| EBNA1_66 | GGRRKKGGWFGKHRGQGGSN | LMP1_43 | DNTDDNGPHDPLPHSPSDSA | BZLF1_19 | AVVFACPGANQGQQLADIGV |
| EBNA1_67 | GWFGKHRGQGGSNPKFENIA | LMP1_44 | PHDPLPHSPSDSAGNDGGPP | BZLF1_20 | GANQGQQLADIGVPQPAPVA |
| EBNA1_68 | GQGGSNPKFENIAEGLRALL | LMP1_45 | SPSDSAGNDGGPPQLTEEVE | BZLF1_21 | LADIGVPQPAPVAAPARRTR |
| EBNA1_69 | KFENIAEGLRALLARSHVER | LMP1_46 | NDGGPPQLTEEVENKGGDQG | BZLF1_22 | QPAPVAAPARRTRKPQQPES |
| EBNA1_70 | GLRALLARSHVERTTDEGTW | LMP1_47 | LTEEVENKGGDQGPPLMTDG | BZLF1_23 | PARRTRKPQQPESLEECDSE |
| EBNA1_71 | RSHVERTTDEGTWVAGVFVY | LMP1_48 | KGGDQGPPLMTDGGGGHSHD | BZLF1_24 | PQQPESLEECDSELEIKRYK |
| EBNA1_72 | TDEGTWVAGVFVYGGSKTSL | LMP1_49 | PLMTDGGGGHSHDSGHGGGD | BZLF1_25 | EECDSELEIKRYKNRVASRK |
| EBNA1_73 | AGVFVYGGSKTSLYNLRRGT | LMP1_50 | GGHSHDSGHGGGDPHLPTLL | BZLF1_26 | EIKRYKNRVASRKCRAKFKQ |
| EBNA1_74 | GSKTSLYNLRRGTALAIPQC | LMP1_51 | GHGGGDPHLPTLLLGSSGSG | BZLF1_27 | RVASRKCRAKFKQLLQHYRE |
| EBNA1_75 | NLRRGTALAIPQCRLTPLSR | LMP1_52 | HLPTLLLGSSGSGGDDDDPH | BZLF1_28 | RAKFKQLLQHYREVAAAKSS |
| EBNA1_76 | LAIPQCRLTPLSRLPFGMAP | LMP1_53 | GSSGSGGDDDDPHGPVQLSY | BZLF1_29 | LQHYREVAAAKSSENDRLRL |
| EBNA1_77 | LTPLSRLPFGMAPGPGPQPG | LMP1_54 | DDDDPHGPVQLSYYD | BZLF1_30 | AAAKSSENDRLRLLLKQMCP |
| EBNA1_78 | PFGMAPGPGPQPGPLRESIV | LMP2_1 | MGSLEMVPMGAGPPSPGGDP | BZLF1_31 | NDRLRLLLKQMCPSLDVDSI |
| EBNA1_79 | PGPQPGPLRESIVCYFMVFL | LMP2_2 | PMGAGPPSPGGDPDGYDGGN | BZLF1_32 | LKQMCPSLDVDSIIPRTPDV |
| EBNA1_80 | LRESIVCYFMVFLQTHIFAE | LMP2_3 | SPGGDPDGYDGGNNSQYPSA | BZLF1_33 | LDVDSIIPRTPDVLHEDLLNF |

TABLE 2
Detailed list of protein antigens on arrays

| Antigen | Product Name | residues | source |
|---|---|---|---|
| BALF2 = EA-p138 = DBP, | EBV Early Antigen P138 | mid to | |
| Major DNA-binding protein | | C-term | |
| BALF5 | Epstein-Barr Virus BALF5 Protein (Recombinant His + SUMO) (aa1-210) | AA1-210 | E. Coli |
| BALF5 | Recombinant Epstein-Barr Virus BALF5 Protein (1-210 aa), His-SUMO-tagged | AA1-210 | E. Coli |
| BCRF1 = EBV-IL10 | BCRF1 recombinant protein :: Viral interleukin-10 homolog (BCRF1) | AA24-170 | E. Coli |
| | Recombinant Protein | | |
| BCRF1 = EBV-IL10 | Recombinant Viral EBV IL-10 Protein Summary | AA26-170 | E. Coli |
| BDLF3 | Recombinant Epstein-Barr Virus BDLF3 Protein (29-186 aa) | AA29-186 | E. Coli |
| BFRF3 = SCP = p18 | EBV Capsid Antigen P18 | full-length | E. Coli |
| BHRF1 = EA-R | Viral BHRF1 protein | AA1-142 | E. Coli |
| BHRF1 = EA-R | Recombinant Epstein-Barr virus Apoptosis regulator BHRF1(BHRF1), partial | AA1-142 | E. Coli |
| BLLF1 = gp350 = gp220 | Human BLLF1 protein | full-length | E. Coli |
| BLLF1 = gp350 = gp220 | Epstein-Barr virus (Herpesvirus 4) EBV Glycoprotein gp350/EBV GP350 | full-length | human |
| | Protein (His Tag) | | cells |
| BLLF1 = gp350 = gp220 | Recombinant gp350/220 (RBD/(a.a.4-450)) (EBV) | AA4-450 | Hek293 |
| BLLF1 = gp350 = gp220 | Recombinant gp350/220 (Ectodomain) (EBV) | AA4-863 | Hek293 |
| BLLF1 = gp350 = gp220 | Recombinant Epstein-Barr virus (Herpesvirus 4) EBV Glycoprotein gp350/EBV | AA1-490 | Hek293 |
| | GP350 Protein (His Tag) | | |
| BLLF3 | Recombinant Epstein-Barr Virus BLLF3 Protein (1-278 aa), His-SUMO-tagged | AA1-278 | E. Coli |
| BLRF2 = p23 | EBV Capsid Antigen P23 | full-length | |
| BMRF1 = EA-D = EA-p54 | EBV Early Antigen P54 | full-length | |
| BRLF1 | Epstein-Barr Virus BRLF1 Protein (Recombinant 10His, N-terminus + Myc, C-terminus) | AA352-605 | E. Coli |
| BRLF1 | Recombinant Epstein-Barr Virus BRLF1 Protein (352-605 aa) | AA352-605 | E. Coli |
| BXLF2 = gH | Recombinant gH(Ectodomain) (EBV) (Strain B95-8) | AA1-679 | Hek293 |
| BZLF1 = EB1 = ZEBRA | Recombinant Trans-activator protein BZLF1 | full-length | E. Coli |
| BZLF1 = EB1 = ZEBRA | Epstein-Barr Virus BZLF1 Protein (Recombinant His + SUMO) (Full Length) | full-length | E. Coli |
| BZLF1 = EB1 = ZEBRA | Epstein-Barr Virus BZLF1 Protein (Recombinant His + SUMO) (Full Length) | full-length | E. Coli |
| CMV Density Gradient | Cytomegalovirus Density Gradient Purified | | NHDF |
| Purified | | | |
| CMV infected Cell Extracts | Cytomegalovirus infected Cell Extracts | | NHDF |
| CMV infected Cell Extracts | Cytomegalovirus infected Cell Extracts | | NHDF |
| CMV purified antigen | hCMV purified antigen | | HFF |
| CMV Purified Glycoprotein | Cytomegalovirus Purified Glycoprotein | | NHDF |
| EBNA1 | EBV Nuclear Antigen EBNA1, P72 | AA72 | |
| | | C-term | |
| EBNA1 | EBV Nuclear Antigen-1 (EBNA-1) Recombinant Protein | full-length | Sf-9 |
| EBNA1 | EBV EBNA1 | AA408-641 | E. Coli |
| EBNA1 | EBV EBNA1, Recombinant | full-length | E. Coli |
| EBNA1 | Recombinant EBV Nuclear Antigen protein | full-length | E. Coli |
| EBNA1 (mosaic) | Recombinant Nuclear antigen-1 (EBV) | AA1-90 + | E. Coli |
| | | AA408-490 | |
| EBNA2 | Viral EBNA2 protein | 208 N-term | Yeast |
| EBNA2 | Recombinant Epstein-Barr virus Epstein-Barr nuclear antigen 2 (EBNA2), partial | AA247-454 | yeast |
| EBNA3 = EBNA-3A = BRLF3 | Recombinant Epstein-Barr virus Epstein-Barr nuclear antigen 3 (EBNA3), partial | AA138 | E. Coli |
| EBNA-3C = EBNA6 | EBNA, Native Protein | full length | native |
| EBV-EA | EBV EA, Native Protein | full-length | P3H3 |
| EBV-EA | EA Protein | full-length | E. Coli |
| EBV-EA | EBV EA protein | AA306-390 | E. Coli |
| EBV Lysate | Epstein-Barr Virus (EBV) Lysate | | Marmoset |
| | | | Leukocyte |
| EBV Density Gradient | Epstein-Barr Virus Density Gradient purified | | P3HR1 |
| purified | | | |
| EBV Infected Cell Extract | Epstein-Barr Virus Infected Cell Extract | | P3H3 |
| EBV Negative Control | Epstein-Barr Virus Negative Control Extract | | Human |
| Extract | | | B Cell |
| gH(DI-III)/gL/gp42 | Recombinant gH(DI-III)/gL/gp42 (Ectodomain)(EBV) (Strain B95-8) Complex | AA1-344 | Hek293 |
| gH/gp42 complex | Recombinant gH/gp42 (Ectodomain)(EBV)(Strain B95-8) Complex | AA1-679 | Hek293 |
| HERV-W | Recombinant Human Syncytin-1 (ERVW-1), partial | AA21-443 | E. Coli |
| HERV-W | ERVWE1 (Human) Recombinant Protein (Q01) | AA116-215 | Wheat |
| | | | Germ |
| HSV1 Infected Cell Extract | Herpes Simplex Virus Infected Cell Extract | | |
| HSV-1 (Macintyre) Density | HSV-1 (Macintyre) Density Gradient Purified | | Vero |
| Gradient Purified | | | |
| HSV2 Infected Cell Extract | Herpes Simplex Type 2 Infected Cell Extract | | Vero |
| HSV-2 (G) Density Gradient | HSV-2 (G) Density Gradient Purified | | Vero |
| Purified | | | |
| LMP1 | Recombinant Epstein-Barr virus Latent membrane protein 1(LMP1), partial | AA185-366 | Yeast |
| LMP1 | Recombinant Epstein-Barr Virus LMP1 Protein (185-386 aa) | AA185-386 | E. Coli |
| LMP1 | Recombinant Epstein-Barr virus Latent membrane protein 1(LMP1), partial | AA185-366 | Yeast |
| LMP2 | Epstein-Barr Latent membrane protein 2 Recombinant Protein Product | 1-148 | Yeast |
| LMP2 | Recombinant Epstein-Barr Virus LMP2 Protein (1-147 aa) | AA1-147 | E. Coli |
| Measles Premium Antigen | Measles Premium Antigen | | |
| NHDF Uninfected Cell Extract | NHDF Uninfected Cell Extract | | |
| NHDF uninfected cell extract | NHDF uninfected cell extract | | NHDF |
| Rubella Premium Antigen | Rubella Premium Antigen | | |
| Rubella Virus Lysate | Rubella Virus Lysate | | Vero |
| VCA, gp125 | Epstein-Barr Virus VCA Protein | full-length | human |
| VCA, gp125 | EBV VCA, Native Protein | full-length | P3H3 cells |
| Vero Uninfected Cell Extract | Vero Uninfected Cell Extract | | Vero |
| VZV Glycoprotein | VZV Purified Glycoproteins | | |
| VZV Lysate | Varicella Zoster Virus (VZV) Lysate | | Strain: |
| | | | Ellen CV-1 |
| VZV Lysate | Varicella Zoster (VZV) Lysate | | Strain: |
| | | | 275 CV-1 |
| VZV Infected Cell Extract | VZV Infected Cell Extract | | |
| VZV Infected Cell Extract | VZV Infected Cell Extract | | NHDF |

TABLE 3

| | | | | | LC | | # |
|---|---|---|---|---|---|---|---|
| barcode_id | HC CDR1 | HC CDR2 | HC CDR3 | LC CDR1 | CDR2 | LC CDR3 | clones |
| MS12_p111w108 | GGSISSGGYY | IYYSGST | CTGHTSSFDYYYGMDVW | QSVSSSY | GAS | CQQYGSSPLFTF | 1 |
| MS12_p111w13 | GFTFSSYA | ISGSGVST | CAKWLYYGSGSYYFYYYGMDV | SSDVGGYNY | EVS | CSSYAGSNNLVF | 1 |
| | | | W | | | | |
| MS12_p111w14 | GFTFSSFG | ISYDGSNK | CAKPGYSGYDFGLGYW | TANIGDNY | DSN | CVTWDSSLSAGVF | 2 |
| MS12_p111w145 | GGTFSSYT | IIPIFGTA | CAPSPAVVAGAMDNDPW | QSISNW | KAS | CQQYDSPPLTF | 3 |
| MS12_p111w15 | GGSLSGYY | ISHSGKT | CARVDYDFWSGFYDSW | SLRSYD | GKN | CASRDISGDHWVF | 1 |
| MS12_p111w153 | GYTFTDYY | ISPNNGET | CARELWSGSPDYYFDSW | SSDIGGYKY | EVT | CTSYAGSNNAVVF | 2 |
| MS12_p111w168 | GDSVSSNSAA | TYYRSKWY | CARERYRNSDVW | QSVSSN | GAS | CQQYNNWPALTF | 1 |
| | | N | | | | | |
| MS12_p111w26 | GYTFGSYD | MNPNSGNT | CARGRLDRNWFDPW | SSDVGSYNL | EVS | CCSYATSSSVVF | 1 |
| MS12_p111w35 | GYTFTSYA | INTNTGNP | CARVMGATKWNDAFDIW | SSDIGGYK* | EVT | CSSYAGSNNAVVF | 1 |
| MS12_p111w69 | GYTFTDYY | INPNSGET | CARELWSGYTDYYFDSW | SSDIGGYKY | EVT | CSSYPGSNNTVLF | 2 |
| MS12_p115w113 | GFTFSSSA | ITGSGDST | CANSDWGAYDSW | QSVSSSD | GAS | CQQYGSSTWTF | 1 |
| MS12_p115w17 | GGSLSGYY | ISHRGKT | CARVNNDFWSGFYDSW | SLRSYD | GKN | CASRDISGDHWVF | 1 |
| MS12_p115w176 | GFTFSTYE | ISSSSSTI | CGRGGYSFDYW | QGIRND | AAS | CLQHNTYPRTF | 1 |
| MS12_p115w3 | GGSLSTYY | ISHRGKT | CARVDNDFWSGFYDSW | SLRSYD | GKN | CASRDISGDHWVF | 1 |
| MS12_p115w48 | GGSLSGYY | ISHSGKT | CARVDYDFWSGFCDSW | SLRSYD | GKN | CASRDISGDHWVF | 1 |
| MS12_p115w7 | GYSISRAYY | IYHDGSP | CARRACSSISCYVDYW | SLRNYY | DKN | CHSRDSSGNHVVF | 1 |
| MS12_p115w8 | GFTFSSYG | IWYDGSNK | CVRDLRQRLVDDDFDMW | QSLVYSDGNTY | KVS | CMQGTQPWTF | 1 |
| MS20_p126w106 | GFTLRHHD | YATAADT | CVTAGVNSNYYGMDVW | QSVSNNF | DAS | CQQHSRSPRGYTF | 4 |
| MS20_p126w111 | GGSVTSVGHY | FYYSGNT | CARIIPLNYLGGPFDSW | QDIRNY | EAS | CQQFDNLPITF | 1 |
| MS20_p126w122 | GFPFSNYW | IKEDGSQK | CARVSTSKWGHFAYW | QNINTF | AAS | CQQTFTTPMSSF | 1 |
| MS20_p126w127 | GGSISSGGYY | IDYSGSN | CARGPSRGWSGRSGNNWFDPW | HDITKY | DAS | CQQYDSLPVTF | 1 |
| MS20_p126w15 | GGSISSSSYY | IYYSGNT | CARRAQYSGSSPYWYFDFW | QSVSSSY | GAS | CQQYGSSPQYTF | 1 |
| MS20_p126w158 | GFTFSAYA | ISDDGSSK | CAKDYYDSGGFYIFDSW | RSLLHKNQYHF | LAF | CMQTLQTPGTF | 1 |
| MS20_p126w162 | GGSISSSSYY | VYFSGRT | CARDHGDYTLHSFYYGMDVW | QSLLHSNGYNY | LGS | CMQALQSPPTF | 1 |
| MS20_p126w164 | GFTFTNYW | INLDGSEK | CARNRAAAGDYW | QDISNY | DAS | CQQYDNVILTF | 3 |
| MS20_p126w174 | GDSITSYY | IFYSGST | CARGTVSGYDLKVSFDYW | QSVLYSSNNKNF | WAS | CHQYHSTPLTF | 2 |
| MS20_p126w18 | GFVFPNYW | IKKDGSEK | CARDVGYCSDPSCYADWFDPW | QSVSRF | GAS | CQQYGTSPRTF | 1 |
| MS20_p126w180 | GGSISSDY | IYYSGIT | CARARQPQHLDYW | QDISNY | AAS | CQQYDNVPFTF | 3 |
| MS20_p126w94 | GGSISGYY | IFASGNT | CVRDGGDTVTRAYDFW | QSISNY | AAS | CQQSYTTPRTF | 1 |
| MS20_p128w126 | GFSLRTSGMC | IDWDEEK | CARENSAYDWGRGRVFDYW | QSIDNW | KAS | CQQYNSYSRTF | 1 |
| MS20_p128w13 | GFPFSNYW | IKEDGSQK | CARVSSSKWGHFAYW | QNINTF | AAS | CQQTFTTPMSSF | 1 |
| MS20_p128w169 | GFSLTTNGMC | INWDDEK | CARIRGPYDAFDIW | QTIYTW | KAS | CQQYNIYTWTF | 1 |
| MS20_p128w177 | GGSFSGYY | INHSGST | CARHLKDPSIAVLIENTFDIW | QSLLHSNGYNY | LGS | CMQILQTPRTF | 1 |
| MS20_p128w2 | GGSVTSVGHY | FYYSGNT | CATIIPLNYLGGPFDSW | QDIRNY | EAS | CQQFDNLPITF | 1 |
| MS20_p128w8 | GGSMVSGGYF | IDSSGST | CARRYYNFWSGYTRNWFDPW | HDISNY | DAS | CQHYENLPPSCAF | 1 |
| MS20_p129w111 | GFSLNTRSVG | IYWEDDK | CAHRRDTIIRGVADAFNFW | QSIDRY | KSS | CLEYNTYSPWAF | 1 |
| MS20_p129w135 | GFTVSSNY | IYSGGST | CARDSLAAAGFTTYYFDYW | QSVSSY | DAS | CQQRSNWPPYTF | 1 |
| MS20_p129w159 | GGSISSYY | IYYSGST | CARSSYYYYGMDVW | TGAVTSGYY | STS | CLLYYGGALVF | 1 |
| MS20_p129w25 | GFTFSSYW | INNDGSFT | CVRDFVPNSNWLDPW | SSDVGSYNY | DVS | CSSYTTSSTWVF | 1 |
| MS21_p26w101 | GGSVNNGGYY | IYFTGNT | CARGFIGYDTDGRDVAANLDS | SSDVGAYKF | DVS | CSSYSTTGLSVF | 3 |
| | | | W | | | | |
| MS21_p26w104 | GFSLSNSRMG | IFSNGEK | CARVQYNSGSYFRDYYDFW | QSISDY | AAS | CQHSYNFPPTF | 19 |
| MS21_p26w106 | GFSFSSYG | ISPSGDTT | CAKDQWELVVFDYW | QSIDTW | KAS | CQRYDSYPWTF | 29 |
| MS21_p26w107 | DGSFSGYY | ITHSGAT | CAVCVTAVHDAFDLW | QSVLYRSNNKNF | WAS | CQQYYGTPYTF | 1 |
| MS21_p26w108 | GYTFSDFH | VNPYSGDR | CARDFRAGNIKGEFDPW | GSNIGSNS | SNN | CATWDDSQGGFVF | 17 |
| MS21_p26w111 | GGSVNSGGYY | IYYSGST | CARAERTHYYESGEFRAWTTF | QGISSW | AAS | CQQANSFPYTF | 1 |
| | | | DYW | | | | |
| MS21_p26w114 | GGSISSSTYY | IYYSGST | CARTGYYDFWSGSRPFYCYMD | QSVSTW | KAS | CQQYDSYPWTF | 1 |
| | | | VW | | | | |
| MS21_p26w115 | GDSISSGDYY | IYYSGET | CARGPDFWNGDHDGYW | QSLVHSDGNTY | KIS | CMQATQFPYTF | 4 |
| MS21_p26w118 | RFSLTTTGVG | IYWNDDK | CAHLSLVLRFLEYLPLPYYDM | QSLVHSDGNTY | KIS | CMQATQFPLTF | 1 |
| | | | DVW | | | | |
| MS21_p26w119 | GESFSGYY | INHGGST | CARGGYSVGWYYFHYW | QSVSSN | GAS | CQQYNNWPTF | 1 |
| MS21_p26w126 | GGSFSGYF | MDHGGIT | CARSSYSSGWYGDFDYW | QDISTH | AAS | CQQSYGTPYTF | 4 |
| MS21_p26w127 | GYTFTTYG | ISADTGKT | CARSVLSAKDTGGLYYDYYYY | NSNIGKNF | DDD | CGTWDSRLSAPWVF | 12 |
| | | | YMDVW | | | | |
| MS21_p26w13 | GVTFRNHG | ISYDGRRK | CAGGEEKSYSHGTFDPDPPVH | SSNIGSNY | KTY | CATWDDRLRAWIF | 2 |
| | | | W | | | | |
| MS21_p26w136 | GFTFQNYG | IIWSGGRT | CARAKTPGDFFYYYMDVW | QGTSTY | GAS | CQQYYSSPFTF | 1 |
| MS21_p26w144 | GFNFGTYV | ITGSGSNT | CAKGFEGLVLAGDYYMDVW | QSVSVY | DAS | CQQRSSWPPITF | 2 |
| MS21_p26w146 | GFSFRAYA | ISYDGSNE | CATRFYFDFDYW | KLDDKY | QDD | CQAWDSSIVVF | 4 |
| MS21_p26w162 | GFKFDNYG | LDWNGGSV | CGKDIGLRWGGIDSW | SSNVGGNT | RDD | CLTWDDSLNGWLF | 7 |
| MS21_p26w167 | GFTFSTYT | ISSSSDYI | CARGPTWIPTTDSYYMDVW | ENISRY | AAS | CQQSYSTPLTF | 1 |
| MS21_p26w168 | GDSISSGDYH | ISYSGSA | CARDAGRFRRLRGFPPQDYW | EGIGNS | AAS | CQKYNSVPFTF | 3 |
| MS21_p26w169 | GGSFSVYY | INHSGFT | CAIYSSSSLASYMDVW | SGSIASNY | EDN | CQSYDSSSHVVF | 7 |
| MS21_p26w17 | GGSISSGDYY | IYTSGST | CARALSGSYYVGWFDPW | QDIRKY | DAS | CQQYDNLPLTF | 1 |
| MS21_p26w171 | GFTFSRYG | ISHDGRDK | CAKIDLATTIGGAPMDVW | NIGSKS | YDS | CQVWDSSGDHSYLVF | 17 |
| MS21_p26w176 | GFTFSSFA | ISGSGRGT | CVRYGAITRLSYLDFW | QSVLYSSNNKNY | WAS | CQQYYTTPPTF | 1 |
| MS21_p26w180 | GYTFSDYY | INPYRGGT | CARDYCSSGSCYLGWLDRW | QLGHKY | QDT | CQAWDSSTGVF | 2 |
| MS21_p27w115 | GGSFSGYL | IHHSGGA | CARLPTVLRGVPGGGRSSIDV | SLRTYY | GKN | CNSRDSRGNNVIF | 6 |
| | | | W | | | | |
| MS21_p26w21 | GFTFSNYA | ISGSGGTT | CAKWLMGAERSLTGHW | QSISSW | KAS | CQQYDSYPHTF | 1 |
| MS21_p26w22 | TFSSYG | ISHDGSEK | CAKGLVYFGWGSPQYYYYMEV | QDINNY | AAS | CQQYKTYPLTF | 3 |
| | | | W | | | | |
| MS21_p26w26 | GYSFADYG | ISAYSGNT | CARDWGDYYGSRSSHDYW | QSISTW | KAS | CQQYNSYSLPWTF | 1 |
| MS21_p26w36 | AFIFSSYP | ISHDGRKE | CVREGLNYADVW | QSISSD | GAS | CQQYNDWPPITF | 3 |
| MS21_p26w43 | GFIFSTHP | ISYDGNNK | CAREAIYYYDSSGYHTATDAF | QSIRSY | AAS | CQQSYTTPYTF | 2 |
| | | | DMW | | | | |
| MS21_p26w51 | GFTFSN*A | ISGSGGTT | CAKWLMGAERSLTGHW | SSNIGANY | SNN | CATWDDSLSGWVF | 1 |
| MS21_p26w57 | GGSISSGGYY | IYYSGST | CARLRRWLQPYSFDIW | SNDVGGYDY | DVS | CCSYADSYTLVF | 1 |
| MS21_p26w58 | GYTFTNYA | INTDNGNT | CARVGRTLGYCSGGSCETGYE | QSISSY | AAS | CQQSHSIPYTF | 5 |
| | | | HYYFMDVW | | | | |
| MS21_p26w6 | GGSIGSSNW | IYHSGST | CARAFVMVTHYYMDVW | SSNIEDNT | SND | CAAWDDTLSRYVF | 2 |
| MS21_p26w62 | GYTFSDYF | INPRTGGT | CARDRPAAGTNYYFYIDVW | QSLDSNGYNY | LGS | CMQSLRTPLVF | 2 |
| MS21_p26w7 | GFTFSSYG | ISGGSTYI | CARDRVGAAAPFDYW | QTISTS | AAY | CEQTYNMPRTF | 1 |
| MS21_p26w8 | GFAFSSYW | IKEDGSEK | CAKCSARTCYPWEECYHYYYY | QSVSSSY | GAF | CQQYVTSVSTF | 1 |
| | | | LDVW | | | | |
| MS21_p26w82 | GFIFSDYN | VSSAGNYI | CARFSPTRLLDYW | DSNIGVNY | RNN | CAAWDDSLSGFVIF | 3 |
| MS21_p26w92 | GGSINSGGHY | MYNSGNI | CARENDFWSGDYTGGFDLW | QTIRSNF | DTS | CQQYGSSPKTF | 1 |
| MS21_p26w93 | GGAFRNCG | IIPLFGMI | CAGGSSAHNSGYIYGDIGAFD | SSDVRAYDY | DVS | CCSYAGSYTLIF | 2 |
| | | | IW | | | | |
| MS21_p26w94 | GFTFVDYA | IGSKVYGG | CTRRYSGSYSRW | QGISNY | AAS | CQQYNSYPITF | 1 |
| | | TT | | | | | |
| MS21_p27w108 | GGSISTYY | IYYSGST | CARPRRPDLWSGYYDAFDIW | QSISTW | KAS | CQQYDNYWTF | 2 |
| MS21_p27w109 | GFIFSSYH | ISHDGRNE | CAREGVAYMDVW | QSVSSD | GAS | CQQYNNWPPLTF | 4 |
| MS21_p27w112 | GFSLTTSGVG | IFWDDDK | CVHEQGGWFGQSRTSRYYYYM | QDISSF | AAT | CQQSYDTPRTF | 1 |
| | | | DVW | | | | |
| MS21_p27w114 | SLTLSIDD | SPSASTT | PAIFRVLEYLGGRILDF | QSVSSSY | GAF | CHQYVTSVYTF | 1 |
| MS21_p27w118 | GYTFTGYF | INPRNGAT | CARDRPSAGTNYYFYIDVW | QNLHINGYNY | LGS | CMQALRTPLTF | 10 |
| MS21_p27w119 | GGSISDYY | IYYTGST | CARGLGIVGTTTKLEFW | QSLLHNNGYKY | LGS | CMQGLQTPWTF | 1 |
| MS21_p27w122 | GGSITSGNW | MYHSGST | CARDVHTIGSGNSRGYMDVW | QSVLYSSNNKNY | WAS | CQQYYSIPWTF | 1 |
| MS21_p27w126 | GLTVSTNY | LYSGGKT | CATEAYDTSGGREVW | GSNIGSNY | WNN | CAAWDDSLSGRVF | 1 |
| MS21_p27w129 | GGSFSGYY | IHHSGRT | CARAPRARTESIAARMGDAFD | GSDVGSYNY | DVS | CSSYTSSTTDYVF | 1 |
| | | | IW | | | | |
| MS21_p27w13 | GGSINSGGYS | IYQSGNT | CARAPSSSSWGYFDLW | QSIGSS | YAS | CQQSRSLITF | 1 |
| MS21_p27w130 | GFAFSSFA | MSGTGGSR | CARDDGQWSTTWYHW | HSISDY | AAS | CQQSDSTPWTF | 1 |
| MS21_p27w14 | GFSFTTYW | INDDGSYT | CVRESLGSGNRYFELW | QSFNNW | KAS | CQEYNSWTF | 1 |
| MS21_p27w143 | GGSISSSGYS | IYDSGST | CAKDNWDYYDSSGYYGAFDIW | QGISSY | AAS | CQQLNSYPHPF | 1 |
| MS21_p27w146 | GDSINGAGYY | ISSSGSS | CAKTYSRSWAYFDSW | QSVRAN | GAS | CQQYNHWPFFTF | 3 |
| MS21_p27w15 | GDSISGGGYS | IYNSGST | CARGGITVFGVIVPCLDPW | QSVSGS | DAS | CQHHNNWPPTFTF | 2 |
| MS21_p27w158 | RFNFNNYA | INYSDDST | CAKPTYEPSGYYGFDIW | QSISRY | GTS | CQQTYTAPLTTF | 1 |
| MS21_p27w163 | GASITSGNW | MYHSGST | CARDVHTIGSGNSRGYMDVW | SSNIGNNY | DNN | CGTWDSSLSGGVF | 1 |
| MS21_p27w167 | NYW | ISSDGNRI | CARTEERRLGEYVYYYYYYMD | GSDVGSYNY | DVS | CSSYTSSTTDYVF | 1 |
| | | | VW | | | | |
| MS21_p27w169 | GGSISSADYY | MYYSGST | CARVWSKGYYSGYFDPW | QDIRNY | DAS | CQQYGNLPLTF | 2 |
| MS21_p27w171 | TGSVSSGGHY | VYYRGSP | CARGLYYYARGKGEIWHFDLW | QSVLTRSNNKNY | WAS | CQQYYSTPITF | 20 |
| MS21_p27w172 | GGTFKKSA | IISTFGAA | CARDMSEQLIPDHYYFYYMDV | QNLRSN | GAS | CQQYNTWPRTF | 2 |
| | | | W | | | | |
| MS21_p27w174 | GFTINNYW | INSDGSTT | CVRDLYGDHPWYMDVW | QSLLYVNGYNY | LGS | CMQALQTPYTF | 4 |
| MS21_p27w176 | GYRFTNYW | TYPGNSDT | CAKFLKSEVLNARDYFDDW | QGIRSY | SAC | GQRTYNAPPSF | 1 |
| MS21_p27w178 | GFTFSDYW | IKEDGSEK | CARGQVWLPYW | QSLLHSNGNNY | MGS | CMQALQTPHTF | 4 |
| MS21_p27w18 | GDSISSGDYY | IYYSGET | CARGPDFWNGDHDGNW | QSLVHSDGNTY | KIS | CMQATQFPYTF | 3 |
| MS21_p27w2 | GGSIGSGGYY | IYYSGST | CARCPRGGSNFIATGLWFDTW | QSVLTRSNNKNY | WAS | CQQSYTPLFTF | 1 |
| MS21_p27w21 | NNY | IYADGST | CAREWKGDFSGYYSFYYYYYM | NFVITS | RDS | CQVWDSTTDHRVF | 4 |
| | | | DVW | | | | |
| MS21_p27w22 | GGSIGSGGYY | IYYSGST | CARCPRGGSNFIATGLWFDTW | QSVSSN | GAS | CHQYNNWLSLTF | 1 |
| MS21_p27w25 | GYSHTFYW | IYLDDSHT | CARAPGSLSYFDNSGHHRADS | QSVLYNSNNKNY | WAS | CQQSYTPLFTF | 3 |
| | | | FDIW | | | | |
| MS21_p27w3 | GYKFTDYH | INPYSGDR | CARDDGTLTFFDNW | SSNIGTNP | TNN | CAAWDDSLSVYVF | 1 |
| MS21_p27w35 | RYPLTELS | FDPEDGDT | CVASHLLAVGHLLPDYW | QTISKW | KAS | CQQYNTYPYSF | 4 |
| MS21_p27w39 | GDSINLYNYY | IFYSGT | CARHRGTAGYYYYSMDVW | QSLLHSNGHHY | LGS | CMQALQTITF | 1 |
| MS21_p27w51 | GFSFSSFA | ISPAGGST | CAKDLGGWELPLGNGFDVW | QGVTRW | AAS | CQQANSLPYTF | 2 |
| MS21_p27w57 | GFTFSSFA | ISYHGRNK | CAKGRGGDGTSVFYFDYW | QDITNY | DVS | CQHYANLPLTF | 1 |
| MS21_p27w58 | GGSVSSSIYY | VYHSGST | CARLTGEYDFWSGSEYYFDRW | QNILYGSNNKNH | WAS | CQQYYSGPPTF | 1 |
| MS21_p27w69 | GFIFDDYA | INWNSRTP | CVKDIGVGVGAMGVAFEHW | QSVSTY | DAS | CQQRINWPPYTF | 1 |
| MS21_p27w8 | GGSISSSSSY | IYYSGSA | CARDEGFGSGHFYTW | RILLHSDGHNY | LGS | CMQALETPLTF | 1 |
| MS21_p27w92 | GLSLSDYT | ISYDGREK | CATDRKGLFPNYYYSHHLDVW | EGISNW | DAS | CQQTHSFPTF | 1 |
| MS21_p28w101 | GFTFSSFA | ISYHGRNN | CAKGRGGDGTSVFYFDSW | QGISNL | DVS | CQHYANLPLTF | 5 |
| MS21_p28w112 | GDSIRDHY | IYYSGST | CAAYSYANWLDPW | QSVSSY | DAS | CLQRRNWPLTF | 1 |
| MS21_p28w115 | GFSVSSSY | IYSGGSI | CARGLGNDHDISGHWGWFDSW | QRISTW | DAS | CQQSNSFPLTF | 1 |
| MS21_p28w118 | GYTFTGHH | INPHSGDT | CARDVGGSDFLSGFDYW | RSNIGSNS | RNN | CATWDDGRSAFVF | 1 |
| MS21_p28w129 | GFKFDDYG | LDWNGGSV | CGKDIGLRWGGIESW | SSNVGGNT | RDD | CLTWDDSLNGWLF | 2 |
| MS21_p28w133 | DYNFADYY | IDPSDSYT | CARRWAVKNRGLMGYYYSYYM | TSNVGSHP | TND | CSTWDDSLNGPVF | 1 |
| | | | DVW | | | | |
| MS21_p28w135 | GFTFISSA | ISGSGGGK | CAKTREFSSGWPGASFDYW | QSVSSSY | GAS | CQQYGSSPLFTF | 2 |
| MS21_p28w145 | GGSINSSSDY | IYSSGTT | CAKANPDVTYSFGYMDSW | QSVTSN | GAS | CQQYNNWPPWTF | 1 |
| MS21_p28w153 | GFTFDDYA | ISWDGGTI | CAKVKTPRPNFYNYFLPETEF | QGISKW | AAS | CQQANRFPLTF | 1 |
| | | | YFDHW | | | | |
| MS21_p28w158 | EFTFDDYS | ISWNSATS | CAKGSGRNWNYGAFEYW | NIGRQS | YDS | CQVWDSSSDHAVF | 3 |
| MS21_p28w178 | GFTFSRYP | ISYDGVIK | CARDWGIAAAGPYYYMDVW | QSVSTY | DAS | CQQRDTWPPVF | 2 |
| MS21_p28w183 | GFTFDDYA | ISWDGGTI | CAKVKTPRPNYYNYFLPETEF | QGISKW | AAS | CQQANRFPLTF | 2 |
| | | | YFDHW | | | | |
| MS21_p28w2 | GGSINNYNDY | IEYRGST | CARHVPTLTDGWYLFRSGFDH | QTVLYSSNNKNY | WAS | CQQYYSTPLTF | 4 |
| | | | W | | | | |
| MS21_p28w22 | GFPFSDYG | IWFDGTKE | CARGRYVLPDAFDIW | ISNIGSNS | GNY | CSAWDDSLSAVLF | 3 |
| MS21_p28w25 | GDSISSGGYY | IYYTGRT | CARAPRGFLEEKYFDSW | QSVLYSSNDKNY | WAS | CQQYFTSPTF | 1 |
| MS21_p28w26 | AGSTSNNNW | IYHSGIT | CARAWFGEFRGAFDIW | QSVSSNY | GAS | CQQYGDLFRTF | 1 |
| MS21_p28w39 | GDSISTTRYY | IYFTGSA | CARHPLPVYYFDSW | QSISTW | KAS | CQQYIRSSWTF | 1 |
| MS21_p28w43 | GFTFSSYA | IWYDGSYK | CAKGVSSGGNTHFDSW | QSISSSY | SAS | CQQYGSSPFTF | 1 |
| MS21_p28w6 | GFSFNAYW | IKQDGSEK | CARDIETSALYKDVLTGFRYF | QSISSW | KSS | CQQYNSYSRTF | 2 |
| | | | YFYYMDVW | | | | |
| MS21_p28w69 | GFTFSTYV | ITGSGSNT | CAKGFEGLLLAGDYYMDVW | QSVSSY | DAS | CQQRSNWPPITF | 1 |
| MS21_p28w7 | DDSISSGGYY | IYYTGRT | CARAPRGFLEEKYFDSW | QSVLYSSNDKNY | WAS | CQQYFTSPTF | 2 |
| MS21_p28w81 | TFTSCG | ISTYTGDT | CARDQRHCSSSWCPFDHW | QSVSSSY | GAS | CQQYGSSKLTF | 1 |
| MS21_p29w10 | GFVFSTCG | ISIDASKT | CARDCHVIDYDFWDASFDSW | SSNIGINT | DKS | CAAWDDSLNGWVF | 1 |
| MS21_p29w109 | GFMFATYG | IWYDGSNK | CATSIPSTGITGALDLW | QSIRSNY | DIS | CQHYGRSPPITF | 7 |
| MS21_p29w13 | GFTFSSYW | IKQDGNEK | CARAVFLEWLLSSYFDYW | QGISNY | ASS | CQNYNSAPLTF | 1 |
| MS21_p29w130 | GGSFSGYY | IHHSGRT | CARAPRARTESIAARMGDAFD | QSVSSN | GAS | CQQYHNWPLTF | 1 |
| | | | IW | | | | |
| MS21_p29w158 | GFSLTNHRMG | IFSNDEK | CVRMYSKGGYPMDYW | QSMSTY | EAS | CQQSYSFPYTF | 1 |
| MS21_p29w159 | GGSISSGDFY | IYYGGTT | CARDQEAAGGTAGNYFDPW | QTIDNF | GAS | CQQSFSSPWTF | 1 |
| MS21_p29w169 | TFSTFA | IRQDGTKT | CARELPYYDSSGHWSERGGFD | TNDFGDYNY | GVT | CSSYMTGSSYVF | 2 |
| | | | LW | | | | |
| MS21_p29w17 | GYTFTGYY | IKPNSGGA | CARGDTYTPSSRYYYYYMDVW | SGSIASNY | EDN | CQSYDSSSHVVF | 1 |
| MS21_p29w183 | GYTFTGYY | IKPNSGGA | CARGDTYTPSSRYYYYYMDVW | ALPKQY | KDS | CQSADSSGAGVF | 1 |
| MS21_p29w21 | GLTVSGQY | IYTVGQT | CAGPSDRYRYYLDVW | QGITKY | AAS | CQKYDSAPLTF | 1 |
| MS21_p29w26 | GGSISSSNW | IYHSGST | CARGRGLRLEDLASFDSW | QSISSY | DAS | CQQNYRVPYTF | 2 |
| MS21_p29w35 | GFTFSTHW | IKQDGSEQ | CARAPYDFWGAYLGSYNYYMD | QSVLYSSNNKNY | WAS | CQQFYSTPYTF | 1 |
| | | | VW | | | | |
| MS21_p29w36 | GGSFSDHG | IVPIFATP | CATRVRVIGRLDASDVW | QSISTW | KAF | CQQYNSYSSTF | 2 |
| MS21_p29w5 | GFTFSDHY | IRKKVHSY | CTRTLSYEPPDHW | QGISNY | AAS | CQQYKTYPLTF | 1 |
| | | ST | | | | | |
| MS21_p29w55 | GFKFDNYG | LDWNGGGV | CGKDIGLRWGGIDSW | SSNVGGNT | RDD | CLTWDDSLNGWLF | 1 |
| MS21_p29w59 | GFGLSDYT | ISYDGREK | CATDRKGLFPNYYYSHHLDVW | EGISNW | DAS | CQQTHSFPTF | 4 |
| MS21_p29w81 | GFTFSSYW | IKQDGSEK | CARAVTIFGGVSPPDYW | QGISNY | AAS | CQKYNSAPWTF | 1 |
| MS21_p29w83 | GDSINSGGYY | IYHTGRT | CARGHYYDSTGYYLPPYYFDY | QSVSSN | DTS | CQQRSNWLTF | 2 |
| | | | W | | | | |
| MS21_p29w89 | GFAFNNYA | ITASAGGT | CAKHQYDLWSAYDYW | QSVSGSS | DAS | CQQYGSSPWTF | 3 |
| MS21_p29w92 | GDSISSGGYS | VYRSGST | CARGLIAARRLDWFDPW | QGISNY | DAS | CQQYHDLPITF | 1 |
| MS21_p30w107 | GYTFNSYN | MNPNSGNT | CARMDVRLASVLSGW | KLGDNY | QDF | CQAWDSSTAVF | 1 |
| MS21_p30w109 | GGSISSSSYY | IYYSGST | CAREDSSSWYPVFYYYYYMDV | SSNIGSNY | RNN | CAAWDDSLSGPVF | 1 |
| | | | W | | | | |
| MS21_p30w114 | GFNFNTHA | INSNGGAT | CVKSPQSGWYYFDSW | QGIGNL | GAS | CQNYRSGAFTF | 1 |
| MS21_p30w13 | SVLP | ISLDK | CARPYWRHDRVLRHHLIF*LD | QTVSSNF | DSS | CQQYGSSPITF | 1 |
| | | | RPL | | | | |
| MS21_p30w143 | GASISNGGYY | IYYSGIT | CAILSLEEGYCSSISCSSDYW | QDISNS | DTS | CQQYDNLPRYTF | 2 |
| MS21_p30w158 | GDSISSGEYY | IYYSGET | CARGPDFWSGDHDGYW | QSLLHSDGNTY | KIS | CMQATQFPYTF | 1 |
| MS21_p30w17 | FTFSSYW | IKQDGSEG | CARDDSREERKFDFWRGYRDY | QSISRW | KAS | CQQYHTYSRTF | 1 |
| | | | YYYYMDVW | | | | |
| MS21_p30w171 | GYTFISYG | ISSRTGKT | CFRHFYDDRNNLGSLDHW | ALPKKF | EDD | CFSTEDTGTDDVTWVF | 1 |
| MS21_p30w175 | GFTFSGYV | ISYDGSNK | CAKTLTRGRRFADGFDIW | SSNVVSNS | SDN | CAVWDGSLSGVLF | 1 |
| MS21_p30w18 | GGSVSSGGHY | IYITGVT | CARGEMGGSPPENW | QSVSSSF | GAS | CHQYDTSPWTF | 5 |
| MS21_p30w22 | GYRFTNYW | TYPGNSDT | CAKFLKSEVLNARDYFDDW | SGSVSTSYH | NTN | CVLYMGSGISIF | 2 |
| MS21_p30w26 | GASVSSGGYY | IYNSGTT | CARDSGWLPLGELSFSRYFDY | QNINNY | AAS | CQQSHSTPWTF | 1 |
| | | | W | | | | |
| MS21_p30w3 | SYW | IDPSDSYT | CARLQRRRFGSSSWWAGSDYY | SGHSTYA | VNSDGSH | CQTWGTGILVF | 1 |
| | | | YYIDVW | | | | |
| MS21_p30w30 | GFSFSSYG | ISPSGDTT | CAKDQWELVVFDYW | QSISNW | QAS | CQRYDSYPWTF | 1 |
| MS21_p30w36 | GFTFNDYA | IIPVLNRS | CARAVSGTYYYYYMDIW | QSISTY | AAT | CQQSYSNPQTF | 1 |
| MS21_p30w48 | GFTFSSYN | INGSGGTT | CARENYYYYYMDVW | QGINNR | DVS | CLQHKDISGFTF | 1 |
| MS21_p30w57 | GFSFNIYA | ISGSGSKA | CAKVLGYCRGDSCYLAMNLEE | QSVSTY | DAS | CQQSYRIPLTF | 2 |
| | | | IAFDIW | | | | |
| MS21_p30w62 | GFIFSNSW | IKQDGSEK | CARAYSSSGTGFDYW | QSISSW | KAS | CQQYNSYPVTF | 1 |
| MS21_p30w82 | GGSISSYH | IHMSGRT | CARESPEGQSLYYHPYYMDLW | NSNIGRNS | RDN | CGTWDDSLDGWVF | 1 |
| MS21_p31w100 | GVSIGTGTYS | IFYSESA | CARMDESAPGGLQNWFDPW | QSVLYSSNNKNY | RAS | CQQYYTTPLTF | 1 |
| MS21_p31w109 | GYTFTDYY | INPNNGGT | CAREGYCSAGSCTDVNWFDPW | SSDIGGYNY | DVN | CSSYARDKWVF | 1 |
| MS21_p31w111 | GYTFTGYF | INPRNGAT | CARDRPSAGTNSYFYIDVW | QNLHINGYNY | LGS | CMQALRTPLTF | 2 |
| MS21_p31w126 | GFTFSRFA | ISSDGGST | CVTPRSVVTGSPIDYW | ALPKKY | EDS | CYSTDSSDNFVF | 1 |
| MS21_p31w129 | GGSISSRDYY | IYHSGST | CARVTSNGVNWFDPW | QSISTY | AAS | CQQSYSIWTF | 1 |
| MS21_p31w136 | GFTFSNYG | MSYDGTKQ | CAKAGRAPPGTSTRDQKNIYY | SSNIGNNL | RND | CATWDDSLSAWVF | 1 |
| | | | HYMDVW | | | | |
| MS21_p31w143 | GHPLTDYY | INPNNGGT | CAREGYCSAGSCTDVNWFDPW | SSDVGGYNY | DVS | CCSYAGSYVVF | 1 |
| MS21_p31w144 | DDSISRGSFY | ISYSGST | CARGDRFNWNLSYYFNYW | QDISNY | DAS | CQQYDNLPLTF | 1 |
| MS21_p31w171 | GFSFRSYA | ISGSGGTS | CAKGSPHEGLLSVGELLYDFD | SSNIGDNF | DNS | CGTWDSSLSEWVF | 1 |
| | | Y | W | | | | |
| MS21_p31w172 | GFTFSSHW | INGDGSST | CARDRKLELLSYYMDVW | QTVSKS | DAS | CQQRSKWPPTF | 1 |
| MS21_p31w175 | GFTFSTYA | ISGSAGST | CAKDSGHIYYYFYYMDVW | SSNIGAGSD | VNS | CQSYDTSLSGFYVF | 1 |
| MS21_p31w2 | GGSIRPYY | ISYNGNT | CARRSLEGFCTNGVCSEDYCL | QYVSIN | GAS | CQQYNRSPQTF | 1 |
| | | | DVW | | | | |
| MS21_p31w22 | GFTFSSYG | ISYDGSNK | CAKERLRFLEWYPGRHAFDIW | QSISSY | AAS | CQQSYSTPITF | 1 |
| MS21_p31w25 | GYIFTGYH | INPKNGGT | CARDPGFSEFLSMADHW | QGIDNY | AAS | CQQLNSYPPLTF | 1 |
| MS21_p31w39 | GFTFDDYG | ISWNSGIM | CAKDGGIRFLEWSTSRPFHYM | SSNIGAGYD | GNN | CQSSDSSLSVVL | 1 |
| | | | DVW | | | | |
| MS21_p31w44 | GFTFSDFP | LSHDGTSP | CARASLTTMHYHYYLDVW | QSIGTY | ASS | CQQSFNSLWTF | 1 |
| MS21_p31w46 | GGSISSSNYY | ISYSGST | CARQISILDGSETSYVHPYYY | TSNIGSNP | NNN | CAAWDDRLTGSWVF | 1 |
| | | | YYMDVW | | | | |
| MS21_p31w58 | GYTFSNFG | VNPYTGNT | CAREKVALAASTTVPDAFHIW | QSISHW | KAS | CQQYHIFSPTF | 1 |
| MS21_p31w59 | GFTVSNNA | FTDTGGST | CAKTRVDYDILTGYYRYFDHW | QSISRW | KAS | CQQYKSYSYSF | 1 |
| MS21_p31w62 | GFTFTTYP | ISYDGNEQ | CARVLSSAWPLSSPDYW | QSVSTY | DAS | CQQRSSWPPALSF | 1 |
| MS21_p32w10 | GGSISSGNYY | IYDTGST | CARAGGVNWFDPW | QSIINY | TAS | CQQSYGSPWTF | 1 |
| MS21_p32w101 | GFFFKNYW | IKLDGSEK | CAREVGVTGTTPLDYW | NIGSKS | YNN | CQVWDDVSGRVF | 2 |
| MS21_p32w107 | GGSITSGDYY | TFDSQRT | CARGPGGDFDYW | SGINVDTYR | YKSDSDN | CMIWHGSAWVF | 1 |
| MS21_p32w158 | GDSVSSGSYY | VSYSGST | CARLRDYYDSRNYYYPKTAFD | QGIRNY | GAS | CQQSYSTPRTF | 2 |
| | | | YW | | | | |
| MS21_p32w159 | GVSLRKSRMG | IFSNGKK | CARVQYNSRSYFRDYYDFW | QSISDY | AAS | CQHSYNFPPTF | 1 |
| MS21_p32w162 | GDSVTSTDYY | IYYSGSP | CARGFNYDDYQSKW | QSINNW | KAS | CQQYNTYSRTF | 1 |
| MS21_p32w177 | GFSFSTYS | IGKNGNYI | CVRCHGVMATICYFDRW | QSISSW | KAS | CQQYTGTSRTF | 1 |
| MS21_p32w18 | GGSFSGYL | IHHSGGA | CARLITILRGVPGGGRSSIDV | SLRSYY | GKS | CNSRDSRGNSVIF | 1 |
| | | | W | | | | |
| MS21_p32w2 | GFTFNRYA | LSGRTGST | CARERDSSSSFDYW | QDISNF | DAS | CHQYENLPYTF | 1 |
| MS21_p32w20 | GFSLSTSGMC | IDWDDDK | CARIAPGTNCYTDYFDYW | QSISSYY | GTS | CQQYGSSRTF | 1 |
| MS21_p32w26 | GGSFSGYY | INHSGST | CARGSYSWYYYW | QSVSSY | DAS | CQQRSNWPTF | 1 |
| MS21_p32w37 | GGSINTGDYY | TFYSGST | CARVGARVVLTTTRGGFDIW | SIDIVSHNY | EVT | CSSYGGGNNLEVF | 1 |
| MS21_p32w39 | GDSINGAGYY | ISSSGSS | CAKTYSRSWAYFDSW | QSVRAN | GAS | CQQRSNWLTF | 1 |
| MS21_p32w43 | GFIFSDYT | ISYDGREK | CATDRKGLFPNYYYSHHLDVW | EGISNW | DAS | CQQTHSFPTF | 1 |
| MS21_p32w5 | GFTVLSSY | LYSGGST | CARDLGSGFDPFKYW | QSFSTW | KAS | CQQYNSYPYTF | 1 |
| MS21_p32w55 | GDSVSSNSAS | TYYRSKWYN | CAREERGVTIFGVIITRLYYM | ALPKQY | KDT | CQSADSSGTYVF | 1 |
| | | | DVW | | | | |
| MS21_p32w93 | GGSISSNNYF | VFYNGGA | CARHIRFADTSSWNYFDYC | QGISSY | AAS | CQQLNSYVFTF | 1 |
| MS21_p33w107 | GFSLSNSRMG | IFSNGEK | CARVQYNSGSYFRDYYDFW | QSISDY | AAS | CQHSHNFPPTF | 1 |
| MS21_p33w111 | GFSFRGYV | ISYDGSNK | CARAKNRWELLRW | QSVSSN | GAS | CQQYDNWPPITF | 1 |
| MS21_p33w112 | SFSSFA | ISFDGSSK | CAKDRFLWGGNDITSWPIDYW | QDISNY | DAS | CQQYDYLPLTF | 1 |
| MS21_p33w133 | GGSVNSRTYY | MFYTGSP | CARYAPYDHAWETYEAFDIW | QSLLQSNGYNY | LGF | CMQALQTPYTF | 1 |
| MS21_p33w135 | GDSISSNHYY | VFYSGST | CVREVGARNWYFSPRKIDPW | QDISDY | DAS | CQQYDNFPPTF | 1 |
| MS21_p33w14 | GFSLDNPRMG | LFSNDEK | CARFNGLDTPIIGYWYFDLW | QDIDTW | AAS | CLQAVSFPYTF | 1 |
| MS21_p33w146 | GGSISNFY | VFDSGNS | CARVSWRPRKFPGIAVAGFDY | QSLLESNGYNY | LGS | CMQTLQIPWTF | 1 |
| | | | W | | | | |
| MS21_p33w159 | NYG | ISPYNDDT | CARAHRNFYYESRGSAYYFYY | KLGDKY | QDN | CQAWDSSTGVVF | 1 |
| | | | MDVW | | | | |
| MS21_p33w162 | GLSLSTGGMC | IDWDDDK | CARGTLAYCGGDCYSLRPWYF | QTITTY | AAS | CQQSFSTLYTF | 1 |
| | | | DLW | | | | |
| MS21_p33w175 | GFTFSRYG | ISHDGRDK | CAKIDLATTIGGAPMDVW | NIGSKS | YDN | CQVWDSSGDHSYLVF | 1 |
| MS21_p33w183 | GGSVSSSIYY | VYHSGST | CARLTGEYDFWSGSEYYFDRW | QNILYGSNNKNY | WAS | CQQYYSGPPTF | 1 |
| MS21_p33w20 | GGPISSSNW | IYHSGST | CARGRGLRLEDLASFDSW | NIGSKN | RDT | CQVWDSSTVIF | 1 |
| MS21_p33w37 | GGSISSSTYY | GYYSGSN | CARHPYDGSGFYVDQW | SSNIGSNF | RND | CAAWDDSLNVVVF | 1 |
| MS21_p33w69 | GYRFTSHW | IYPADSDV | CARLPGWGGWSPRADFW | QGIGNY | GAS | CQQLNTYTLTF | 1 |
| MS21_p33w93 | GFTFSSFA | ISYHGRNT | CAKGRGGDGTSVFYFDYW | QGIGNL | DVS | CQHYANLPLTF | 1 |
| MS28_p10w109 | GDSMSGYH | IYYSGGT | CARLRRGYSRPKYYYYGMDVW | QSLLHSFGDNY | LGS | CMQALQTPPYTF | 14 |
| MS28_p10w111 | GFSLSTPEMR | IDWDDDT | CARMVRGVAARPRTYYFDYW | NIGSKS | DDS | CQVWDISSDPLGIF | 13 |
| MS28_p10w135 | GGSFTDYT | IRHGGST | CARRASFRTGFYENYSFYYYL | ETIDNY | AAS | CQQSYSLPYTF | 3 |
| | | | DVW | | | | |
| MS28_p10w136 | GYSITGSYY | VYHSGST | CARDLFQGYFGATYQEIDYW | TSNIETNY | RDN | CAAWDDSLSGRVF | 14 |
| MS28_p10w164 | GFSLSNARLG | IFSDDEE | CARTVVRLPDYW | QSILYSSNNKNF | WAS | CHQYYSSPQSF | 1 |
| MS28_p10w17 | GDSVSSTDHS | VNYNGFT | CARGTSGEQFGDYW | EDVITE | GAS | CLQDINNPWTF | 2 |
| MS28_p10w2 | GYTFTSYH | INPSGGST | CARDPLFWSDDYSLGYYYYMD | SSNIGNNY | DNN | CGTWDSSLSDVVF | 1 |
| | | | VW | | | | |
| MS28_p10w44 | GGSIDISGYY | VHHSGSA | CARAHRRTAQIFDSW | ISNIGAGYD | GDN | CQSYDTSLSGPDVVF | 7 |
| MS28_p10w69 | GYIFGTYW | IYPGDSET | CARTDCSRTSCSSLDPW | QSISNW | KAS | CQQYNSFPLTF | 2 |
| MS28_p1w10 | GYTFTSYH | INPSGGST | CARDPLFWSDDYSLGYYYYMD | SSNIGNNY | DNN | CGTWDSSLSDVIF | 1 |
| | | | VW | | | | |
| MS28_p1w14 | GGSVSAYY | ITHSGSP | CARVLINYYYMDVW | QSVSSSY | GVS | CQQYGSSPMYTF | 5 |
| MS28_p1w17 | GSSISNYY | ISDSGTT | CARDRGGISSTWYRNYYYYGL | QSIGNW | QAS | CQQYNNYSPLTF | 1 |
| | | | DVW | | | | |
| MS28_p1w20 | GDSITTYY | TYNSGST | CARDKEHTYGRTFDYW | NSDVGAYNY | DVN | CCSYAGGDVVF | 1 |
| MS28_p1w35 | GGTFSSYA | IIPIFGTA | CARGTIFGVVIRGSGWFDPW | QSLLHGFGANY | LGS | CMQALQTPPYTF | 1 |
| MS28_p1w43 | GDSVNSRRYY | VYSSGST | CARDVGVKGHDFWSGQDHWFF | QSINSW | KAS | CQQYDSYPWTF | 1 |
| | | | DLW | | | | |
| MS28_p1w55 | GFTFISVW | TNTDGSIT | CVRDREALDWFDFW | PASLSSSP | null | CAAWDDSLNGYVF | 1 |
| MS28_p1w57 | GGTFSRYT | IIPIFGTA | CGRVSSDRESQRFGGLFDYW | QSVSSTY | GAS | CQQYGSSSYTF | 1 |
| MS28_p1w8 | RFTFSNYA | ISYDESNE | CARSKGTSSGYDFIFDIW | QTILYSSNNKNY | WAS | CQQYYSTPLTF | 2 |
| MS28_p1w92 | GGSVSAYS | ITHSGSP | CARVLINYYYMDVW | QSVSSSY | GVS | CQQYGSSPMYTF | 1 |
| MS28_p5w109 | GGSFTDYT | IRHGGST | CARRASFRTGFYENYSFYYYL | ETIDNY | AAS | CQQSYSLPHTF | 1 |
| | | | DVW | | | | |
| MS28_p5w114 | GYSFTSYW | IDPSDSYT | CARLGVATILDPWALWGHWYF | QTISTY | AAS | WQQSYITPRTF | 1 |
| | | | DLW | | | | |
| MS28_p5w129 | GFSFDDYT | ITWDGGIT | CTKGGPSTVMFASWHSDLW | YFGTKS | DDS | CQVWDSSSDHVVF | 1 |
| MS28_p5w13 | GFTFTTYW | IKQDGSEK | CARTWIQLWPFDYW | QSVLYSSNNKNY | WAS | CQQYYSTPFTF | 1 |
| MS28_p5w163 | GFIFSSSV | IRGNGDYT | CAKSLPTSRYYFDYW | TSDVGGYDY | DIS | CSSYSGSSTDVVF | 1 |
| MS28_p5w171 | GFTFSSYA | ISGSGYST | CAKSYSTVVTPNDYW | SSDVGNYNL | EVN | CCSFTSSATWVF | 1 |
| MS28_p5w178 | GCDVSAYG | ISPGDSTT | CAKIRQEIISPGLPQPPGLRA | QSVLYSSNNKNY | WAS | CQQHYGNPRTF | 1 |
| | | | FDVW | | | | |
| MS28_p5w193 | GDSMSGYH | IYYSGGT | CARLRRGYSRTKYYYYGMDVW | QSLLHSFGDNY | LGS | CMQALQTPPYTF | 1 |
| MS28_p5w51 | GFSLSTPELR | IDWDDDT | CARMVRGVAARPRTYYFDYW | QGISSY | AAS | CQQYYSYPRTF | 1 |
| MS30_p104w10 | GFSLRSSGVG | IYWDDDR | CARTWLWKDYFDYW | QSISRY | GAN | CQQCRDSVLTF | 2 |
| MS30_p104w112 | GYNFAEFW | IYPGDSET | CARRGGWGRGSYYFDYW | QSVSSNY | GAS | CQQYGSSFTF | 5 |
| MS30_p104w114 | GGSISSSGYS | IHYTGNT | CARGDGWMQLADW | TSNIGDNF | DSN | CGAWDGSLSAGVF | 20 |
| MS30_p104w145 | SGSA | IRSKANSY | CTRHVEMATIYSGNDYW | QSLVYSDGNTY | KVS | CMQGTHWPFTF | 1 |
| | | AT | | | | | |
| MS30_p104w163 | GYTFNNSA | ISVANGNT | CARGQDSDYPYTVFYSNDPEF | ILANKY | KDS | CYSGAGNTRMF | 3 |
| | | | W | | | | |
| MS30_p104w176 | GYTFSTYG | ISGYNGRR | CARAGETDYEFWSGYRYGFDV | SSDVGGHDF | EVT | CSSYAGSNTVVF | 1 |
| | | | W | | | | |
| MS30_p104w20 | GFSLRSSGVG | IYWDDDR | CARTWVWKDYFDYW | QSISRY | GAN | CQQCRDSVLTF | 1 |
| MS30_p104w21 | GFTFSSYW | INHDGSST | CVRDRGYDSSGLWNKMFDYW | SSDIGAGYD | SNT | CQSQDSTLSDVGVVF | 1 |
| MS30_p104w30 | GFTFNTYA | VSFDGTKK | CARDRRSFGGADVLPRDAFDV | RSVFYSSNNKNY | WAS | CQQYYTTYSF | 4 |
| | | | W | | | | |
| MS30_p104w35 | GHTFTNHA | INAGKGNR | CARDTGGWKERDSFDIW | ERINTNT | GAS | CQQYGSSFIF | 1 |
| MS30_p61w115 | GFPFSNFA | IRGSGSNT | CAKTNFGDYYHYGMDVW | QGISNY | AAS | CQQFYSYPLFTF | 1 |
| MS30_p61w118 | GYNFAKYW | IFPDDSDT | CARRPSPSYHYVSGGYSGDVF | HSISSY | DAS | CQHRSNWPPMWTF | 7 |
| | | | DIW | | | | |
| MS30_p61w122 | GFTFRIFA | VSGSGDDT | CAKDQWLGRFDLW | SSDVGGYNY | DVT | CCSYGDTYARNVF | 4 |
| MS30_p61w129 | GFSLTSSGVG | IYWDDDR | CARTWLWKDYFDYW | QSISRY | GAN | CQQCRDSVLTF | 2 |
| MS30_p61w135 | GFKCRDYG | ISYEGRTE | CARDVLGILTGQFDPW | QSVVHSDGNTY | KVS | CTQATQFPFTF | 1 |
| MS30_p61w145 | GASVSSNTVG | TYYRSKWH | CARTQWVGSSLLFDYW | QSISNW | KAS | CQQYDSPPLTF | 1 |
| | | N | | | | | |
| MS30_p61w167 | GYTFTSFD | MNPNSGNI | CARVRGGRYFDYW | QSLLHSHGYNY | LGS | CMQALQTPWTF | 1 |
| MS30_p61w174 | GFTFSRYW | IDQDGSEK | CARTGYSSNSLDYW | SSNIGYNS | DNN | CGTWDSSLSVGVF | 1 |
| MS30_p61w175 | GDSISSGGYY | IYSSGGT | CARGSSSAYLRHGRLGPMDVW | SGSVSTTHY | NTD | CVLYMGSGILVF | 1 |
| MS30_p61w176 | GGSVSSSSHY | MYYKGTT | CARLRIASLPGVAYYGMDVW | SSNIGAAYP | ANN | CQSFDSSLSAEVF | 1 |
| MS30_p61w178 | GFSLSSPTMG | SYSNDEK | CARTEASYGWGSFDSW | RNDVCGYNY | EVS | CGSYADFKNVVF | 1 |
| MS30_p61w180 | GFIFSSYA | ISYDGSSK | CASVWELLSVTGTPDYW | SSDVGGYNY | YVT | FSSYTTSSTLVF | 1 |
| MS30_p61w20 | GGSFNGYS | INHSGDT | CATSMTSFYGFDVW | QSILSSSYNKNY | WAS | CQQYYSNPLTF | 1 |
| MS30_p61w25 | NYG | ISGYNGRR | CARVGETDYEFWSGYRYGFDV | SSDVGGHDF | EVT | CSSYAGSNTVVF | 3 |
| | | | W | | | | |
| MS30_p61w48 | GGSFSGYY | IDHSGTS | CAKQGGNSLIQGGYYFDSW | QSISSW | DAS | CQQYNNYPWTF | 1 |
| MS30_p61w89 | DVSVSSGAYY | VDYRGST | CARYDPPYSFDDW | QGISSY | AAS | CQQLNSYPYTF | 1 |
| MS30_p67w109 | GGSFSGYY | INHSGNT | CATTDDFWSGYFEGLHW | QSLLHSNGYTY | LGS | CMQALQTPLTF | 1 |
| MS30_p67w115 | RFSLSTTGVN | IYWDGDK | CAHSGGYGSGLTW | SSDVGGYDY | DVN | CSSYTSNSTYVF | 1 |
| MS30_p67w123 | GGSISSDDYY | IYYSGST | CARSRNHHSYDGINDYYYYGM | ESVSTSY | DAS | CQQRSSWPPPF | 1 |
| | | | DVW | | | | |
| MS30_p67w127 | GDSVRNNNLY | VYGTGNT | CVGAPAKPNVDWLSPFDYW | HSLGSV | LAS | CQQHNSWPLTF | 1 |
| MS30_p67w129 | GFAFGSYS | ISSHGTDE | CARDSVPYVGVVTRGVYFDIW | QSVATY | DAS | CQQRSSWPPPF | 3 |
| MS30_p67w130 | GFTFSSYG | IWFDGSNK | CARIRDIGGIDYW | GGSLASNY | ENT | CQSYDTGNPWVF | 1 |
| MS30_p67w143 | SFSTYG | ISYDGGKT | CARDSVPYVGIVTRGIYLDSW | QSVSTY | DAS | CQQRSNWPPPF | 1 |
| MS30_p67w145 | GFSLSTTGVN | IYWDGDK | CAHSGGYGSGLTW | QRISRY | AAS | CQQSYGHPPTF | 4 |
| MS30_p67w163 | GITFNPYA | ISASGGST | CVRGRLGYCSGASCEKNWFDP | SNNVGSYA | RDS | CSTWDYSLSALSS | 1 |
| | | | W | | | | |
| MS30_p67w167 | GYTFTTYY | INPSGGRT | CARGRTAFYMDVW | QSVATY | DAS | CQQRSSWPPPF | 1 |
| MS30_p67w169 | GFTFSTYA | IVGSANT | CAKGWATFRVDITNDAFDVW | QSLLHSDGRTY | AVS | CMHTIQLPYTF | 1 |
| MS30_p67w20 | GITVRSYA | ISASGGST | CVRRRLGYCSGASCEKNWFDP | SGSVSTSYY | STN | CVLYMGSGISVF | 1 |
| | | | W | | | | |
| MS30_p67w39 | GYTFTDYL | IHPENGNT | CARALSPSGSGWNLAHW | LGISNY | AAS | CQKYNSFPLTF | 1 |
| MS30_p67w48 | GCTFRSSA | ISYDGSTQ | CARASSTGGDHIAAVRLGDYW | QSVSSN | GAS | CQQYNDWPRTF | 1 |
| MS30_p67w51 | GFTFSSYG | ISFDGSNK | CAKDHLPHYYISSGYFDHW | QDIRGW | AAS | CQQGNSFPRTF | 4 |
| MS30_p67w8 | GFTFSNYW | IKVDGSEE | CARVRGWYDYFDCW | QSLLHSNGHHY | LAS | CMQALQTRTF | 1 |
| MS30_p67w89 | GITFNTYA | ISASGGST | CVRGRLGYCSGASCEKNWFDP | SGSVSTSYY | STN | CVLYMGSGISVF | 1 |
| | | | W | | | | |
| MS30_p67w92 | GYRFTNYD | MSPDSGNT | CARGGDWFDLW | RSDVGGYNY | EVT | CSSYAGTNIHVLF | 1 |
| MS30_p67w93 | GFTFSNYA | IVGSANT | CAKGWATFRVDITNDAFDVW | QSISNY | AAS | CQQSYITPVTF | 2 |
| MS30_p70w115 | GGSISSDY | IYNSGTT | CARGGPHYGDYGALWDYW | QSIKSY | AAS | CQQSYNTPWTF | 1 |
| MS30_p70w127 | GGTFSDYG | IIPMFHTL | CARAPLRSRSNWELLLKRVDF | QSISSNF | DAS | CQHYGSSPPFTF | 2 |
| | | | LYLDVW | | | | |
| MS30_p70w130 | GGSFSGDY | INRSGST | CARGLAAMAPYGLDVW | QVINSY | AAS | CQQLNSYPFTF | 1 |
| MS30_p70w136 | GYSFTGYY | INPNSGGT | CARDLAPAAISGYYHYGMDVW | SSDVGGYNY | YVT | CSSYTTSSTLVF | 2 |
| MS30_p70w171 | GVSISSNKY | IYHSGSL | CASYCTGLSCYIDSW | ALPKQY | KDS | CQSADSSGSYVVF | 1 |
| MS30_p70w175 | GDSISSGGYY | IYSSGST | CARGSSSAYLRHGRLGPMDVW | QSVLNRSNNKNY | WAS | CQQYYSPPLTF | 2 |
| MS30_p70w177 | GFTFRSYA | VSDDGSII | CARATTRNGSSFPHFDYW | QSIKSY | AAS | CQQSYNTPWTF | 1 |
| MS30_p70w21 | GVSISSNKY | IYHSGSL | CASYCTGLSCYIDSW | QDISHY | AAS | CQQYHTYPLTF | 1 |
| MS30_p70w48 | GGSISSGAYY | IYYTGST | CARAPISARYFDLW | QSISSW | DAS | CQQFYSYPLFTF | 1 |
| MS30_p70w69 | GFTVSSNY | IYSGGST | CARARDYYHSSGYDYW | SFNIGNNY | DNN | CGTWDSSLSAWVF | 1 |
| MS30_p70w81 | GFTLRRYG | ISHDGTTT | CAKDLYGYDSSGVYISIDLW | SSDVGGFNY | DVG | CSSYTTSSAVLF | 1 |
| MS30_p73w10 | GFTFSSHD | ISGSGGTI | CAKRRVGTYPAHDYW | QSVTSHY | GAS | CQQCGSSFCTF | 1 |
| MS30_p73w108 | GLIVSNNY | ISAGGDT | CARGWFQLPRDWFDPW | QSVSNY | DAS | CQQRSNWPLTF | 2 |
| MS30_p73w111 | VESFSDYY | INGRGDS | CARGLNWNFFSWYFDLW | QSISSF | SAS | CQQSYITPVTF | 2 |
| MS30_p73w126 | GYNFAKYW | IFPDDSDT | CARRPSPSYHYGSGGYSGDVF | HSISSY | DAS | CQHRSNWPPMWTF | 5 |
| | | | DIW | | | | |
| MS30_p73w136 | GYIFTNYW | IDPSDSYA | CARRGQGVLSSSDIW | QGTRNY | GVF | CQQYNIHPWTF | 2 |
| MS30_p73w15 | GGTFNSFS | IVPMIDKT | CARLTVVVTAMSHYYINGMDV | QSISSSF | ATS | CQQYFGLPPITF | 2 |
| | | | W | | | | |
| MS30_p73w158 | GGSISSDDYY | IYYSGST | CARSRNHHSYDGINDYYYYGM | ESVSTSY | GAS | CQQYGRSPITF | 3 |
| | | | DVW | | | | |
| MS30_p73w174 | GFTFSGSW | IKPDGSAR | CARGYLW | QSISSW | DAS | CQQYNYYF | 2 |
| MS30_p73w22 | GDSISSAGYY | ISYGGSA | CARDNDYGDLLDYW | QSISRY | TAS | CQQSYSSPRSF | 1 |
| MS30_p73w30 | YTFSSYG | ISGYNGRR | CARAGETDYEFWSGYRYGFDV | SSDVGGHDF | EVT | CSSYAGSNTVVF | 1 |
| | | | W | | | | |
| MS30_p73w51 | GYIFTNYW | IDPSDSYA | CARRGQDLLSSSDIW | QGTRNY | GVF | CQQYNIHPWTF | 2 |
| MS30_p73w57 | GGSISSSNW | IYDSGST | CARLYGLGSSDDLW | ALPKQY | KDS | CQSADSSGTYWVF | 1 |
| MS30_p73w62 | GGALSTYA | IIPILVTP | CARGSTDTNTGFFDYW | QSVHSNY | DAS | CQQYGDSISF | 1 |
| MS30_p73w82 | GYTFSSYG | ISGYNDRR | CARAGETDYEFWSGYRYGFDV | SSDVGGHDF | EVT | CSSYAGSNTVVF | 1 |
| | | | W | | | | |
| MS30_p75w111 | GVSITSANW | IYRSGST | CVRDLHTIFESEDQW | QSVFYSPNNQNY | WAS | CQQYYTTPLTF | 1 |
| MS30_p75w113 | GFTFSSYA | IDGSGGST | CAKRYGDGAFDIW | QSISSW | DAS | CQQYNSYPITF | 1 |
| MS30_p75w158 | GFTFSNAW | IKSKSAGG | CTTDQGIADRPSIGYW | QSVSSN | GAS | CQQYNNWPLTF | 1 |
| | | TT | | | | | |
| MS30_p75w163 | | ISASGGST | CVRGRLGYCSGASCEKNWFDP | SGSVSTSYY | STN | CVLYMGSGISVF | 1 |
| | | | W | | | | |
| MS30_p75w171 | FSLSDFGEG | IYWDDDK | CAHRMRSGIRFFDYW | QGISYY | DAS | CQQRSEWPPLTF | 1 |
| MS30_p75w183 | YNFTTYW | VDPSDSYT | CARRRLSGYSLDAFSLW | QKVGSN | DAV | CQQYSGWPPEGTF | 1 |
| MS30_p75w25 | VLTFPTFG | VSVSGDST | CAKRYYYESSGYYYEPGDAFD | QSVSSN | GAS | CQQYNNWPRTF | 1 |
| | | | IW | | | | |
| MS30_p75w35 | GGSFSGYY | INHSGST | CARGRGLLEWLFHYYFDYW | QSISYY | AAP | CQQSYNTPRTF | 1 |
| MS30_p75w59 | GFTFSSHT | ITSSGAYK | CARDALTTIFGVTANTYAMDV | NSNVAGIY | RTN | CAAWDDNLSGQVF | 2 |
| | | | W | | | | |
| MS30_p75w89 | GFSLNNARMG | IFSKDEK | CAREMSATGGYWFFDIW | QSISTY | AAS | CQQCYSSPRSF | 1 |
| MS30_p80w107 | GGSVTSGGYF | IYDSGST | CARAGFKGEYPEFIQLW | SSNVGSHT | NNN | CGAWDDSLNGPVF | 1 |
| MS30_p80w174 | GFTFSSYW | INSDGSST | CARVLPSGSLAADYW | SSDVGSYNF | EVS | CCSYVGSSLYVF | 1 |
| MS30_p80w18 | NGSIISTVYH | IYFTGNS | CARQAQETSGWTRDWYFDLW | ALPKKY | EDS | CYSTDSSSNQRVF | 1 |
| MS30_p80w180 | GFALSSSA | IVVGSSNT | CAAEEYQRAHAGYW | QSLVHSDGNTY | RIS | CMQATQLRTF | 1 |
| MS30_p80w2 | GVSVNSADSN | IHYSGNV | CARGRGEYEGFDVW | QSVLYSPNNRNH | WAS | CQQYYSSPFTF | 1 |
| MS30_p80w20 | GYTLTELS | FDPKDRET | CAAEYLAPRTGTFDYW | QAISRA | DAS | CQHFDSYAQTF | 1 |
| MS30_p80w48 | YGSIRSHSYF | IYVTETT | CARHRGNGPAGITTGMDVW | RSNIGTNT | SDD | CAAWDDNLNGLWAF | 1 |
| MS30_p80w62 | GYTFTSYG | ISAYNGNT | CARDSAFSSGSLFNGVFDIW | QSVSSSY | GTS | CQQYGSSPWTF | 1 |
| MS30_p87w100 | GYSFTTYA | INTGSANT | CAREKATRRGNYVYYYGMDVW | QDISNY | DAA | CQQYDTLPYSF | 1 |
| MS30_p87w113 | GFTFSSYA | ISFDGSNK | CARGEVVVTAALFEHW | RSNIGRTY | SNN | CAAWEDTLSAHVVL | 1 |
| MS30_p87w118 | GYNFAKYW | LFPDDSDT | CARRPSPSYHYVSGGYSGDVF | HSISSY | DAS | CQHRSNWPPMWTF | 1 |
| | | | *YL | | | | |
| MS30_p87w130 | GFSLNNARVG | IFSNDVK | CARMHEYCSTTTCYTDFYYGM | QSLVHSDGNTY | MVS | CMQGTHWPVTF | 1 |
| | | | DVW | | | | |
| MS30_p87w153 | GFTFGTYT | ISYDGSNT | CARDSVPYVGVVTRGVYIDYW | QSVDSY | DAS | CQQRSNWPPPF | 1 |
| MS30_p87w158 | GFSFNSYG | ISKDGGTK | CAKDYDFWGGPGETTDPW | SSDVGGYNY | EVS | CSSYAGSNNLVF | 1 |
| MS30_p87w174 | GGSFTGYY | INHSP | CARGPPTYYHDNSGYYFFDYW | SSAIGDYNY | DVS | CSSYSSSSTLVFVF | 1 |
| MS30_p87w24 | HTCA | VSFDGTKK | CARDRRSFRGADVLPRDAFDV | RSVFYSSNNKNY | WAS | CQQYYTTYSF | 1 |
| | | | W | | | | |
| MS30_p87w25 | LASFSDYY | INGRGDS | CARGRNLNFFSWYFDLW | QSISSF | SAS | CQQSYITPVTF | 1 |
| MS30_p87w3 | GGTFSSYG | IIPMFGTT | CARARGYSWDDAFDIW | QSVSSSH | GAF | CQQYGSSVTF | 1 |
| MS30_p87w55 | TFSNYW | INRDGDKK | CAVDQALRGMPSIEGWFDPW | QSLVHSDGNTY | MVS | CMQGTHWPVTF | 1 |
| MS30_p87w6 | GGSVTSHF | VNYGGRA | CARGSGQYCTNGVCYPEVFDF | QRVTNTY | GAS | CQQYGSSPPISF | 1 |
| | | | W | | | | |
| MS30_p87w8 | SNSAS | TYYRSTWS | CAKVVKDDHGWYANPFDIW | QSISSN | AAS | CQHSFTLPYTF | 1 |
| | | T | | | | | |
| MS30_p87w89 | GFTFGDYA | LRSKAYGG | CTRQRLAAVGYFDNW | NIGSKS | YDN | CQVWDSSSDHLWY | 1 |
| | | TT | | | | | |
| MS30_p92w100 | GGSINTNSYY | IDYSGST | CAGHTPLYYFDASTYQEDYW | SSNIGSNT | SCN | CAACDDSLSGPFGC | 1 |
| MS30_p92w108 | GFTFLPFS | INRDGTEE | CARAPIYFYDTPGPFDYW | QSVLHRSNHNNY | WAS | CQQYYSALITF | 1 |
| MS30_p92w129 | GGSIISNYYY | IYFSGST | CARQAYCSSTACYKFDFW | QSLLHSNGYNY | LGS | CMQSLQTPVTF | 1 |
| MS30_p92w146 | GGSFSRYY | INESGST | CANSGRITVTSVNW | QSISYY | AAS | CQQSYNTPRTF | 1 |
| MS30_p92w15 | GYTLRSYG | ISAYTGKT | CARGYGDRPWFDTW | QNVLYSSNNKNY | WAS | CQQYYTAPPHTF | 1 |
| MS30_p92w162 | GYSFTSYW | IDPSDSYT | CARKGGDTTGLLDHW | QSVRTN | GAA | CQQYNKWSTF | 1 |
| MS30_p92w35 | GGSFSWYY | INESGST | CANSGRITVTSVNW | QSISYY | AAS | CQQSYNTPRTF | 1 |
| MS30_p92w47 | GFTFSKNG | ISGSGGST | CAKDRGRDYYDFWSGTYYFDY | RSNIGSND | DTN | CGAWDSSLNAGYVF | 1 |
| | | | W | | | | |
| MS30_p92w57 | GGSLSESLYS | IFHSGAL | CAKYDFIDRYNPLGWFDPW | QSLSGNH | RAS | CQQYDFPPLTF | 1 |
| MS30_p92w89 | GYSISSNNW | IHHSGST | CARGDTYYASGAFDYW | QSLLHSNGYNY | LGS | CMQALQSYTF | 1 |
| MS30_p94w100 | GVSITTNSSY | IEYSGST | CAGHTPLYYFDASTYQEDYW | QDISNY | AAS | CQHLDSYPITF | 1 |
| MS30_p94w101 | GFTFRTYA | ISFDGSNK | CARGEVVVTAALFEHW | RSNIGRTY | SNN | CAAWEDTLSAHVVL | 1 |
| MS30_p94w14 | GFTFGSYS | ISSHGTDE | CARDSVPYVGVVTRGVYFDIW | QSVATY | DAS | CQQRSSWPPPF | 2 |
| MS30_p94w153 | GYTFTSYG | ISAYNGNT | CARCLMHYYDSSGYYYHDAFD | QSMGSY | GAS | CQQSYSIPRTF | 1 |
| | | | IW | | | | |
| MS30_p94w162 | NYTFTCQG | VIGYNGKT | CARVAAVAGIDFW | QRVRTN | GAA | CQQYNKWSTF | 1 |
| MS30_p94w163 | GYSFSDFG | ISAHNGYT | CARVVRGSGSFFYYYYGMDVW | QSISSNF | GAS | CQQYGTSPWGF | 1 |
| MS30_p94w164 | GGSVSSDNYY | IYYSGNS | CARDRYDSRGFYGVDSW | KLGDKY | QDY | CQAWDSSTKVF | 1 |
| MS30_p94w167 | GYSFATHW | IHPGDSET | CARRLSTPYYYNFVMDVW | SSHVGTYNL | EGS | CCSYARSRSDVVF | 1 |
| MS30_p94w180 | GGSISRDNYY | IYYSGST | CARESDPYGSGSFTW | QSISRW | DAS | CQQYNSYSRTF | 1 |
| MS30_p94w21 | GFTLSDHF | TRNKANRY | CARGGKGGAFDIW | QSISKY | AAS | CQQSYSTQWTF | 1 |
| | | TT | | | | | |
| MS30_p94w5 | GFPFSAFY | ISGRNIYT | CVRESLQGPAFEFDYW | QAINNN | AAS | CQQYKSSPPTF | 1 |
| MS30_p94w51 | GYTFTSYG | ISAYNGNT | CARTPMVRGVVFDYW | QSISSW | DAS | CQQYNSYSTF | 1 |
| MS30_p94w7 | GYTFSSYG | ISGYNGRR | CARAGETDYEFWSGYRYGFDV | SSDVGGHDF | EVT | CSSYAGSNTVVF | 1 |
| | | | W | | | | |
| MS30_p94w93 | GYSFTSYW | IYPGDSDT | CARPGGGYGYWYFDLW | SSDVGGYNY | DVS | CSSYTSSSTYVF | 1 |
| MS30_p99w101 | GGTFSNYA | IIPLFGTA | CARGFQKRYSSSWYYVWFDPW | SLRSSY | GKN | CNSRDSSGNHLGELF | 1 |
| MS30_p99w123 | GFTFRNFA | IRGSGSNT | CAKTNFGDYYHYGMDVW | QGISNY | AAS | CQQFYSYPLFTF | 1 |
| MS30_p99w13 | GYSLRTHW | IYPGDSHT | CASGDYYDSSGYPEYW | SGDVGGSNY | DVT | CSSYTSSSTYVF | 1 |
| MS30_p99w143 | GGTFSDYG | IIPMFHTS | CARAPLRSRSNWELLLKRVDF | QTISNNF | DAS | CQHYGSSPPFTF | 1 |
| | | | LYLDLW | | | | |
| MS30_p99w145 | GFTFSMFG | MSYDEIKE | CAKGWPGDSGADAFDVW | QSISNY | AAS | CQQSFSTPLTF | 1 |
| MS30_p99w162 | NYTFTGYG | VIAYNGKT | CARVAAVAGIDFW | QDIINN | AAS | CLHNHNYPRTF | 1 |
| MS30_p99w167 | GYSFSTYW | VYPGDSDT | CARHTGRNDYW | NRDVGGYNY | GVN | CGSFTSSGTLYVF | 1 |
| MS30_p99w171 | GFTFSGSA | IRSKSNSY | CCGQNYDNYYYAMDVW | QGISRH | AAS | CQQLISYPPITF | 1 |
| | | TT | | | | | |
| MS30_p99w21 | VFTFSSYW | INHDGSST | CVRDRGYDSSGLWNKMFDYW | SSNIGSNY | SNN | CAAWDDSLSGWVF | 1 |
| MS30_p99w22 | GFTFRSYW | MNQDGSEK | CARDKAYGDSHEYW | QSISRY | AAS | CQQSSTTPWTF | 1 |
| MS30_p99w44 | GGSISSRNFF | VYYTGSA | CARHPYCTNGVCYPSRLYW | QSISSSY | AAS | CQQYGSSPPFTF | 1 |
| MS30_p99w48 | GGSFSDSY | ISDSGSI | CARARTRQVVIPGSSSGFHPP | QSLMQTNEYKY | LGS | CMQTLQTPRTF | 1 |
| | | | PYSFYYFGMDVW | | | | |
| MS30_p99w57 | GFTFSNNV | ITSNGGST | CVRFCSGDSCYPRW | SSDVGAYNY | DVN | CCSFAGSYTWVF | 1 |
| MS30_p99w6 | TFSSYE | ITTSGSTA | CARWKDAVMGTQSNWFDPW | QSVRSY | DAS | CQHRISWPLTF | 1 |
| MS30_p99w93 | GGSFSGYY | INHSGST | CARGWVRIVGATHFDYW | SSDVGGYNY | DVS | CSSYTSSSTLVF | 1 |
| MS30_p99w97 | GFPFSTYS | INNSSSYI | CAKERGDIVVEQVANGISISI | PSNIGDNF | SNN | CAAWDDSLNGPVF | 1 |
| | | | TVWTS | | | | |
| MS31_p20w10 | GGSISSFY | IYYSGSA | CARGAEGAFDIW | GSNIGAGFD | GNS | CQSYDSSLSAYVIF | 19 |
| MS31_p20w104 | GFTFSRFS | ITSNGDSI | CARDLPDYIWGTYRPIHFDYW | QSLVHSDGNTY | KVS | CLQATHFPHLTF | 4 |
| MS31_p20w107 | GFTFNSYE | IDTSGDSI | CARHGIHMLRGWFDLW | QSISSS | GAS | CQQYYDWPPLTF | 1 |
| MS31_p20w109 | GYTFTDYY | INPNSGDT | CARAKSAPGHPFYYYYAMDVW | SSDVGDYNY | DFR | CCSYAGTFVVF | 18 |
| MS31_p20w112 | AYKLTL*G | LYRGDYDA | YSTRRYWDLP*GLSP | QAISKY | DAS | CQHYDILPFF | 1 |
| MS31_p20w119 | GYTFTDYF | INPNSGDT | CARAKSAPGHPFYYYYAMDVW | SSDVGDYNY | DFR | CCSYAGTFVVF | 2 |
| MS31_p20w136 | GYTFTDYY | INPNSGDT | CARAKSAPGHPFYNYYAMDVW | SSDVGDYNY | DFR | CCSYAGTFVVF | 4 |
| MS31_p20w14 | GFTFSTYA | ISYDGGNK | CAKDPYSNYGSIDYW | SADIGSHY | KNN | CAAWDDSVTSPNYVF | 4 |
| MS31_p20w158 | GFTFSSYG | VSGSGDSA | CAKDRGYHYGGCDYW | TSDVGGYKY | DVT | CSAYTVSGVVF | 1 |
| MS31_p20w163 | SNSAA | TYYRSKWY | CARVGYSYGLGGDAFDIW | QSISFY | AAS | CQQSYSTPVAF | 1 |
| | | N | | | | | |
| MS31_p20w176 | GGSFSGYY | INHSGST | CARNDFWSGYYPRGWFDPW | HSVTSNY | GAS | CQQYGNSPITF | 1 |
| MS31_p20w193 | STRSRRCF | SWVFLVAL | CARGMHTAIITWDAFDFW | RLGDKF | QDN | CQAWDSRIGVF | 1 |
| MS31_p20w22 | GFTFSTYG | ISGSGDSA | CAKDRGYHYGGCDYW | SSDVGGYKY | DVT | CSAYTVSGVVF | 1 |
| MS31_p20w3 | GGPISRGGYY | IFYTGTT | CARVHGGDWGVYWYFDLW | SSDIGGYNY | DVS | CSSFTSGSTLGVF | 2 |
| MS31_p20w47 | GYTFTHYG | ISGYNGDT | CAREMGDHWAGTHGLDVW | QSLNNY | ATS | CQQTLTAPRTF | 4 |
| MS31_p20w5 | D | MNPNSGNT | CARGVKSSSWYVSGGKYGIHG | SSNIGSNT | SNN | CAAWDDGLNGLPF | 1 |
| | | | MDVW | | | | |
| MS31_p20w57 | GYTFTGYY | INPNSGDT | CASAGGSSGWPQHYFDYW | QSVSSSY | GAS | CQQYGSSPFTF | 1 |
| MS31_p20w59 | GHTFTDYY | VNPNSGDT | CARAKSAPGHPFYYYYAMDVW | SSDVGDYNY | DFR | CCSYAGTFVVF | 1 |
| MS31_p20w6 | AFSFSSYG | ISYDGSNK | CVKDRIVRITTGYYNYGMDVW | QSLLHSNGYTY | WGS | CMQALETPPTF | 4 |
| MS31_p20w62 | GFTFSSYS | ISSSSSYI | CAVGEYYYDSSGYYECYFDYW | ALPKQY | KDS | CQSADSSGTYPRWVF | 1 |
| MS31_p20w69 | GFTFSSYG | VSGSGDSA | CAKDRGYHYGGCDYW | SSDVGRYNF | EVT | CSSYAGSNTFNYVF | 1 |
| MS31_p25w112 | GHTFTDYY | VNPNSGDT | CARAKSAPGHPFYYYYAMDVW | QSLLHSNGYTY | LVS | CMQALQTPPFTF | 1 |
| MS31_p25w143 | GFTFSSFW | MNSEGSSI | CARGTYVSAASMDVW | STDIGEYTF | DVN | CNSYTSRRTVIF | 1 |
| MS31_p25w146 | GGSISGYS | IYYSGAT | CARDRAGYDFDFDSW | ESVSRK | DAS | CQQYSNWPPLTF | 1 |
| MS31_p25w159 | GYTFTDYY | INPNSGDT | CARAKSAPGHPFYYYYAMDVW | SSDVGDYNY | DFR | CQSYDSSLSAYVIF | 1 |
| MS31_p25w167 | GSPFTGYY | INPNSGDT | CAILERLL**ESLLLLPTL | QSLLSSSNNKNY | WAS | CEQYYSSPLTF | 1 |
| MS31_p25w174 | SGSVRSSHYY | IYYSGNT | CATGTYSTDAFEIW | GLPKQY | KT | CQAIDSRDNWVF | 1 |
| MS31_p25w177 | GFILSTYA | FSATSGDS | CARDVAARWSGGFKRKPQYYY | SSDVGRYNF | EVT | CSSYAGSNTFNYVF | 1 |
| | | | AMDVW | | | | |
| MS31_p25w180 | GFIFSSYG | IGYDGSNK | CVRDRMGTNSGSYFFGYW | KLGDKY | QDT | CQAWDSSTGGVF | 1 |
| MS31_p25w183 | GYTFTDYY | INPNSGDT | CARAKSAPGHPFYYYYAMDVW | SSDVGDYNY | DFR | CYSYAGTFVVF | 1 |
| MS31_p25w24 | GFTFTDYA | ISSSGNYI | CVRGVGPTPRFDFW | ERINNY | RAS | CQQYDSFSITF | 1 |
| MS31_p25w5 | GITFSRDG | IWFDGSTK | CARDIFQMSKTVTPNYYGMDV | SSNIGNNF | DNN | CGTWDSSLSAGVF | 1 |
| | | | W | | | | |
| MS31_p25w59 | GFMFSSYG | ILFDGSNQ | CAKGKSGSFIYGMDVW | SSNIGSNY | RNN | CAAWDDSLSGWVF | 2 |
| MS31_p25w69 | GYTFTDYY | INPNSGDT | CARAKSAPGHPFYYYYAMDVW | SSDVGDNNY | DFR | CYSYAGTFVVF | 1 |
| MS31_p25w7 | GGSTSSFY | IYYSGSA | CARGAEGAFDIW | GSNIGAGFD | GNS | CQSYDSSLSAYVIF | 2 |
| MS31_p25w81 | GYTFTDNY | INPNSGGT | CARDAPIRDSNGYSTDYW | SSDVGDYNY | DFR | CSSYAGSNTFNYVF | 1 |
| MS31_p25w93 | GYTFTGYY | INPNSGAT | CARGISAWHTAAFDVW | QSINSRY | AAS | CQQYGSSYTF | 1 |
| MS31_p25w97 | GYTFTGFY | VNPNSGGT | CARDAPIRDSNGYSTDYW | KLGDKY | QDG | CQAWDSSTDVVF | 1 |
| MS31_p30w10 | YG | ISTYNGNT | CAREGRIGHYNDRRRGVYHSY | QSVYTNY | EVS | CQQYGDSPPWTF | 1 |
| | | | YGMDVW | | | | |
| MS31_p30w109 | GGSTSSFY | IYYSGSA | CARGAEGAFDIW | GSNIGAGFD | GNN | CQSYDSSLSTYVIF | 1 |
| MS31_p30w115 | GFMFSDYP | ISRSGGSA | CAKVHDGGNHVPFDYW | QSIGSS | YAS | CHQSSSLPWTF | 2 |
| MS31_p30w129 | GGSISSFY | IYYSGIA | CARGAEGAFDIW | GSNIGAGFD | GNS | CQSYDSRLSAYVIF | 1 |
| MS31_p30w13 | GYTFTDYY | INPNSGDT | CARAKSAPGHPFYYYYAMDVW | SSDVGDYNY | DFR | CCSYVGTFVVF | 1 |
| MS31_p30w130 | GYTFTDYY | INPNSGDT | CARAKSAPGHPFYYYYTMDVW | SSDVGDYNY | DFR | CCSYAGTFVVF | 1 |
| MS31_p30w133 | GYTFTGYY | INPNSGGT | CARAVGVTSYGMDVW | QSISSY | AAS | CQQSYSTPWTF | 1 |
| MS31_p30w143 | GYTFTGYY | INPNSGDT | CAILERLL**ESLLLLPTL | QDISSY | DAS | CQQYDNLPITF | 1 |
| MS31_p30w145 | TFSRFS | ITSNGDSI | CARDLPDYIWGTYRPIHFDYW | QSLVHSDGNTY | KVS | CLQATHFPHLTF | 1 |
| MS31_p30w159 | GYTFTDFY | INPNSGDT | CARAKSAPGHPFYYYYAMDVW | SSDVDDYNY | DFR | CCSYAGTFVVF | 1 |
| MS31_p30w168 | GFPFSNYA | ISGSGDST | CAVSFYDFWSGTDYW | QSVSSY | GAS | CQQRSNWPRTF | 1 |
| MS31_p30w18 | VCSISSFY | IYYSGIA | CARGAEGAFDIW | GSNIGAGFD | GNS | CCSYAGTFVVF | 1 |
| MS31_p30w26 | GGSISSFY | IYYSGSA | CARGAEGAFDIW | GSNIGAGFD | GNS | CCSYVGTFVVF | 1 |
| MS31_p30w37 | GYTFTDYY | VNPNSGDT | CARAKSAPGHPFYYYYAMDVW | SSDVGDYNY | DFR | CCSYAGTFVVF | 2 |
| MS31_p30w46 | GFTFSNYA | ISGSGDST | CAVSFYDFWSGTDYW | QSVSSY | GAS | CQQRSNWPRTF | 1 |
| MS31_p30w62 | GDSISNYH | FYDTGST | CARERPGRADVAYEIW | GSNIGAGFD | GNS | CCSYVGTFVVF | 1 |
| MS31_p30w7 | GYTFTDYY | INPNSGDT | CARAKSAPGHPFYFYYAMDVW | SSDVGDYNY | DFR | CCSYAGTFVVF | 1 |
| MS31_p30w94 | GLTFYSFA | ISGSGGAT | CAQTLEAAFLHSFYRGYFDNW | QSVASNY | GAS | CQQYGSTPLTF | 1 |
| MS31_p35w10 | GFTFSSYA | ISASGSST | CAKDEDSSVVTRPEIDYW | TGAVTSGHY | HTS | CLLSYSGARPVF | 1 |
| MS31_p35w101 | GCLCTLHF | INPNSGDT | CARAKSAPGHPFYYYYAMDVW | SSDVGDYNY | DFR | CQSYDSSLSAYVIF | 1 |
| MS31_p35w108 | GDSISRTTYY | IYYASST | CARVKYYQDSSGYSNWFDPW | QCINTF | AAS | CQQSYSTPLYTF | 1 |
| MS31_p35w115 | GFTFSSYP | IGYDGRIT | CARDPLPGYGDYLDHW | QNILHSSNNKNY | WAS | CQQYYSTPLTF | 1 |
| MS31_p35w145 | GFTFSNYA | IRDDGGST | CAKHWGASYYGSKNTYYYYGL | QSVLYSSNNKNY | WAS | CHQYYDTLQTF | 1 |
| | | | DVW | | | | |
| MS31_p35w15 | GFPFSAYS | ISSSSSYI | CARDYDYVWGSYPTTEYYFDY | SLRSYY | GKN | CNSRDSSGNHWVF | 1 |
| | | | W | | | | |
| MS31_p35w158 | GYTFAAYH | INPSGDTT | CASSTQIKDTGYSTGWYGYW | QSIGSS | YAS | CHQSSTFGGSWTF | 1 |
| MS31_p35w159 | GSSITNGDSY | VYYSGST | CARRAVNRGHERPYDAFDIW | SSDVGSHDL | ALT | CCSYVGRDTLDWGF | 1 |
| MS31_p35w167 | GYTFTGYY | INPNNGGT | CARAGGRDGYKVYFFDYW | QSISSY | AAS | CQQSYSSPWTI | 1 |
| MS31_p35w177 | GYTFTDYY | INPNSGHT | CARAKSAPGHPFYYYYAMDVW | SSDVGDYNY | DFR | CCSYAGTFVVF | 1 |
| MS31_p35w22 | GYTFTGYY | INPNSGGT | CARGYDVFDYW | SGSIASNY | EDN | CQSYDSSNHVVF | 1 |
| MS31_p35w3 | GFTFSSYG | ISYDGSNK | CAKDKAAVVAGYGMDVW | SGSIASNY | EDN | CQSYDTSNPYWVF | 1 |
| MS31_p35w30 | GFTFSTYG | ITGSGDSA | CAKDRGYHYGGCDYW | SSDVGVYNF | DVT | CSAYTVSGVVF | 1 |
| MS31_p35w5 | GFIFNNYA | IWHDGFNK | CARDIVHYSMIDYYNYMDVW | QSVRSRY | GAS | CQQYVSSPPRITF | 1 |
| MS31_p35w62 | GGSISSFY | IYYSGSA | CARGAEGAFDIW | GSNIGAGFD | ANS | CQSYDSSLSAYVIF | 1 |
| MS31_p35w81 | GYTFTDYY | INPNSGDT | CARAKSAPGHPFYYYYAMDVW | SSDVDDYNY | DFR | CCSYAGTFVVF | 1 |
| MS31_p35w89 | GYTFTDYY | INPNSGDT | CARAKSAPGHPFYYYYAMDVW | SRDVGDYNY | DFR | CQSYDSSLSAYVIF | 1 |
| MS37_p13w10 | GGAISSSSFY | LYYSERT | CARDGSTDYGPDWFDPW | QSVRSN | GAS | CQQYTNWLVTF | 18 |
| MS37_p13w104 | GFSFDDYA | IGWHSGTI | CAKSNAAGPYSGSGYAFYFDS | SDIDVSAYN | YDSDSNK | CMIWPSHEGVF | 5 |
| | | | W | | | | |
| MS37_p13w109 | GFPFSNYA | ISRIGDNT | CVSVASTIFGVVFPSIFESW | NIGSKS | DDS | CQVWDTSSEHYVF | 10 |
| MS37_p13w119 | GGPMTRHY | IYTRGTT | CARDAYSSSFWFFDLW | QNIDIY | AAS | CQQSFSTPWTF | 11 |
| MS37_p13w130 | GFTFSSYA | ISNSGGST | CAKASGDFVLGYFQHW | QSVLYSSNNKNY | WAS | CQQYYSTPLTF | 1 |
| MS37_p13w169 | GGPMTRHY | FYTRVTT | CARDAYSSSFWFFDLW | QNIDIY | AAS | CQQSFSTPWTF | 2 |
| MS37_p13w183 | GFPFTNYA | ISRIGDNT | CVSVSSTIFGVVFPSIFESW | NIGSKS | DDS | CQVWDTSSEHYVF | 26 |
| MS37_p13w2 | GFTFNNYA | IRGSGSNT | CAKVTFGDYFQYGLDVW | QAISSY | VAS | CQQFYSFPVTF | 5 |
| MS37_p13w57 | GFTFDNYA | VDGSGAST | CAKVGLDIRVVRGELLTWAFE | QTIDSRY | AAS | CQQYGSTTFTF | 7 |
| | | | YW | | | | |
| MS37_p13w62 | GFPFSNYA | ISRIGDNT | CVSVSSTIFGVVFPSIFESW | NIGSKS | DDS | CQVWDTSSEHYVF | 3 |
| MS37_p13w8 | GFTFSNYA | ISGSGDTT | CVKGLNYVWGSYRGEYYSYGM | QSLLTSNGNNY | LGS | CMHALQTPGF | 5 |
| | | | DVW | | | | |
| MS37_p13w94 | GFPFTNYA | ISRIGDNI | CVSVSSTIFGVVFPSIFESW | NIGSKS | DDS | CQVWDTSSEHYVF | 6 |
| MS37_p14w10 | GFPFTNSA | ISRFGDNT | CVSVSSTIFGVVFPSIFESW | NIGSKS | DDS | CQVWDTSSEHYVF | 3 |
| MS37_p14w106 | GYSFTSYG | ISAYNGNT | CARGTKYAWNVPPEDDYW | QGIRND | AAS | CLQHNTYPPFF | 3 |
| MS37_p14w108 | GGSFSGYY | INDSGGP | CARLIYGDFYYGMDVW | QGISSY | AAS | CQQFNSYPWTF | 8 |
| MS37_p14w111 | GFSLGSSGVG | IYWNDVK | CAHERPYCTSMSCSDYYGMDV | SSNIGAGSD | GDN | CQSSDSSLSGSKVF | 2 |
| | | | W | | | | |
| MS37_p14w126 | GFTFDNYA | VDGSGVST | CAKVGLDIRVVRGELLTWAFE | QTIDSRY | AAS | CQQYGSTSFTF | 5 |
| | | | YW | | | | |
| MS37_p14w146 | GFSLGSSGVG | IYWNDVK | CAHERPYCTRMSCSDYYGMDV | SSNIGAGSD | GDN | CQSSDSSLSGSKVF | 7 |
| | | | W | | | | |
| MS37_p14w158 | GFSLGSSGVG | IYWNDVK | CAHERPYCTRMTCSDYYGMDV | SSNIGAGSD | GDN | CQSSDSSLSGSKVF | 2 |
| | | | W | | | | |
| MS37_p14w162 | YNFRTYW | MYPGDSDT | CARQADAYSWDNRALYDVW | QSVSSN | GAS | CQQYNNWPLLTF | 7 |
| MS37_p14w17 | GFPFTDYA | ISRIGDNT | CVSVSTTIFGVVFPSIFESW | NIGSKS | DDS | CQVWDTSSEHFVF | 11 |
| MS37_p14w172 | GFSLGSSGVG | IYWNDVK | CAHERPYCTRMTCSDYYGMDV | QSVGSD | DIS | CQQYNNWPPWTF | 1 |
| | | | W | | | | |
| MS37_p14w175 | GGSFSGYY | INDSGST | CARGGSGGSGIYYNGPLWRYY | QSISRW | KAS | CQQYSSYPRTF | 1 |
| | | | YGMDVW | | | | |
| MS37_p14w18 | GFPFTNYA | ISRIGDNT | CVSVSSTIFGVVFPSIFESW | NIGSKS | DDT | CQVWDTSSDHYVF | 3 |
| MS37_p14w183 | GESFRGYY | IDHSGST | CARWDGGYRHGSDTYVYYGLD | QSVGSD | DIS | CQQYNNWPPWTF | 1 |
| | | | VW | | | | |
| MS37_p14w3 | GFPFTNYA | ISRIGDNT | CVSVSSAIFGVVFPSIFESW | NIGSKG | DDS | CQVWDTSSEHYVF | 1 |
| MS37_p14w36 | GCTFGNYA | ISGSGETT | CVKGLNYVWGSYRGEYYSYGM | QSLLTSNGNNY | LGS | CMHALQTPGF | 1 |
| | | | HVW | | | | |
| MS37_p14w46 | DGSFNTNY | VNHSGST | CASRDYYDYTWPVDFW | QSVGSD | DIS | CQQYNNWPPWTF | 1 |
| MS37_p14w55 | DGSFNTNY | VNHSGST | CASRDYYDYTWPVDFW | QPITTN | ATS | CQQSHSALMYSF | 1 |
| MS37_p14w6 | GFPLGRSGVG | IYWNDVK | CAHERPYCTSMSCSDYYGMDV | QGISSY | AAS | CQQFNSYPWTF | 1 |
| | | | W | | | | |
| MS37_p14w62 | GYSFTAYW | IDPSDSFT | CATSITPSYYDTVWGTFVPTS | QSVSSN | GAS | CQQYNNWPSPWTF | 5 |
| | | | MDVW | | | | |
| MS37_p14w8 | GEPLTGDY | IDHYGRT | CARGRGDYRTRGVTSYFDRW | QDIEKY | DAS | CQQYEDVPITF | 18 |
| MS37_p14w94 | TFSTYS | ISTTGSTI | CARDPGESSSWYEGVWYFDLW | QGISSNF | DAS | CQQYVTSPTF | 1 |
| MS37_p15w104 | GFPFTNYA | ISRIGDNA | CVSVSTTIFGVVFPSIFESW | NIGSKS | DDS | CQVWDTSSEHYVF | 1 |
| MS37_p15w108 | GFSLGSSGVG | IYWNDVK | CAHERAYCTRMSCSDYYGMDV | SSNIGASSD | GDN | CQSSDSSLSGSKVF | 1 |
| | | | W | | | | |
| MS37_p15w118 | GFPFTNYA | ISPIGDNT | CVSVSSTIFGVVFPSIFESW | NIGSKS | DDS | CQVWDTSSEHYVF | 8 |
| MS37_p15w119 | GESFRGYY | IDHSGST | CARWDGGYRHGSDTYFYYGLD | QSVGSD | HIS | CQQYNNWPPWTF | 1 |
| | | | VW | | | | |
| MS37_p15w126 | GFTFSNYA | ISGSGETT | CVKGLNYVWGSYRGEYYSYGM | QSLLTSNGNNY | LGS | CMHALQTPGF | 5 |
| | | | HVW | | | | |
| MS37_p15w178 | GFTFSSYA | ISGSGGST | CAKGDGYYCSGGSCYSFPDFD | QGIRND | AAS | CLQHNSYPPYTF | 1 |
| | | | YW | | | | |
| MS37_p15w20 | GFPFTNYA | ISRFGDNT | CVSVSSTIFGVVFPSIFESW | NIGSKS | DDS | CQVWDTSSEHYVF | 2 |
| MS37_p15w30 | GGSITSHY | VHYSGST | CGGDSSGWHYFDSW | QSVSRN | GAS | CQQYDNWPLAF | 4 |
| MS37_p15w47 | GFPFTNYA | ISRIGDNT | CVSVSTTIFGVVFPSIFESW | NIGSKS | DDS | CQVWDTSSEHYVF | 3 |
| MS37_p15w94 | GFTFDIYW | INQDGSQK | CAKDSGLYDYAPTMGFDSW | SSDIGAYNY | DVT | CSSYTITSTRVF | 2 |
| MS37_p16w10 | GFPFTNYA | ISRIGDNT | CVSVSSTIFGVVFPSIFESW | NIGSKS | DDS | CQVWDTSSEHFVF | 1 |
| MS37_p16w114 | GFSLGSSGVG | IYWNDFK | CAHERPFCTRMSCSDYYGMDV | SSNIGAGSD | GDN | CQSSDSSLSGSKVF | 2 |
| | | | W | | | | |
| MS37_p16w119 | GFTFDNYA | VDGSGAST | CAKVGLDIRVVRGELLTWAFE | QTIDSRY | AAS | CQQYGSTSFTF | 8 |
| | | | YW | | | | |
| MS37_p16w130 | YG | ISAYNGNT | CARSQPDVLTGYYLNYWYFDL | QSISNY | DAS | CQQRSNWPLTF | 1 |
| | | | W | | | | |
| MS37_p16w153 | TFTVSDFY | IYTGGNT | CARVNNYYFVFDIW | QSVDSRY | GTF | CQQYSYSHTF | 1 |
| MS37_p16w159 | GYTFTGFY | INPNSGGT | CARGYPGFDYW | NIGSKS | DDS | CQVWDSSSDHPVL | 2 |
| MS37_p16w163 | VFSLGSSGVG | IYWNDFK | CAHERPFCTRMSCSDYYGMDV | NIGSKS | DDS | CQVWDTSSEHYVF | 1 |
| | | | W | | | | |
| MS37_p16w164 | GGSISNYY | VYFSGTT | CARMGPEPEYSDSSGTWDYFD | QSISNF | TAS | CQQYSNWPYTF | 1 |
| | | | LW | | | | |
| MS37_p16w167 | GYTFTSYG | ISAYNANT | CARGLALDTAMSGVDYW | QSLVYSDGNTY | KVS | CMQGTHWRF | 2 |
| MS37_p16w176 | GFPFTNYA | ISRIGDNT | CVSVSTTILGVVFPSIFESW | NIGSKS | DDS | CQVWDTSSEHFVF | 2 |
| MS37_p16w183 | GFTFSIYG | ISYDGANI | CAKGRSVMTTEVPDSW | QGISGW | AAS | CQQYSTYPLTF | 3 |
| MS37_p16w47 | GFPFTNYA | ISRIGDNT | CVSVSTTIFGVVFPSIFESW | NIGSKN | DDS | CQVWDTSSEHYVF | 1 |
| MS37_p16w51 | GGPISTSSYY | IYSSGTT | CARPLGTLTHLEDTSSHWFDP | QSISNY | DAS | CQQRSNWPLTF | 1 |
| | | | W | | | | |
| MS37_p17w101 | GDSVSSNSVA | TYYRSKWY | CARGPIDAFDIW | SSNIGSTF | RNN | CAAWDDSLSGWVF | 1 |
| | | N | | | | | |
| MS37_p17w114 | GFPFSNYA | ISLIGDKT | CVSVSSTIFGVVFPSIFESW | NIGSKS | DDS | CQVWDTSSEHYVF | 3 |
| MS37_p17w130 | AFPLGRRGVG | IYWNDVK | CAHERPYCTRMSCSDYYGMDV | STNIGAGYD | DNK | CQSYDRSLNGWVL | 1 |
| | | | W | | | | |
| MS37_p17w25 | GYTFTTFG | ITVYNGNT | CARAWHPDSWFDPW | SSNIGAGYD | GNT | CQSFDSSVSDSAVF | 2 |
| MS37_p17w6 | GFPFSNYA | ISLIGDNT | CVSVSSTIFGVVFPSIFESW | NIGSKS | DDS | CQVWDTSSEHYVF | 6 |
| MS37_p17w82 | GGAISSSSFY | LYYSERT | CARDAYSSSFWFFDLW | QSVRSN | GAS | CQQYTNWLVTF | 1 |
| MS37_p17w97 | GYTFTGHY | INPNSGDT | CARPDDGNSVFDYW | SGHSRYT | LYSDGSH | CQTWGTGTQGWVF | 2 |
| MS37_p18w101 | GASFSGYY | INHREKT | CARALYDDDIWSGPFYYYGMD | QSISDW | KAS | CQQYNNYPYTF | 2 |
| | | | VW | | | | |
| MS37_p18w106 | GYTFNSYG | ISAYNGNT | CARGSGYSAVAEFDPW | QGIRND | TAS | CLQHNSYPPTF | 1 |
| MS37_p18w111 | GDSVSSNSAA | TYYRSKWYN | CASGPTDAFDIW | SSNIGSNY | RNN | CAAWDDSLSGWVF | 2 |
| MS37_p18w119 | GESFRGYY | IDHSGST | CARWDGGYRHGSDTYFYYGLD | QSVGSD | DIS | CQQYNNWPPWTF | 4 |
| | | | VW | | | | |
| MS37_p18w169 | GYSFTGYY | INPHTGGK | CARGARNYGPGYNWFDPW | QSISSW | QAS | CQQYNSDSVRF | 4 |
| MS37_p18w183 | GFSLRTSGAG | IYWNDDK | CAHSPRRYDFRSGYYSLPDVW | SGHNSYA | LNSDGSH | CQTWGTGGGVF | 1 |
| MS37_p18w20 | GFSLGSSGVG | IYWNDVK | CAHERPYCTRMSCSDYYGMDV | SSNIGSNY | RNN | CAAWDDSLSGWVF | 1 |
| | | | W | | | | |
| MS37_p18w26 | GGSFSGYY | INHNENT | CARDFHRPEHHCSIGSCYGFD | QSISTY | AAS | CQHSYNAPYTF | 2 |
| | | | VW | | | | |
| MS37_p18w35 | GFSLTDPTMG | IFSSGEK | CARIRPDQWLVTTSRPSYYFD | ALPKKY | EDN | CYSTDSSGNHGVF | 1 |
| | | | FW | | | | |
| MS37_p18w44 | GFPFTDYA | ISRIGDNT | CVSVSTTIFGVVFPSIFESW | NIGSKS | DDT | CQVWDTSSEHFVF | 1 |
| MS37_p18w59 | GYSFTSYG | ISGDNGNT | CARGTKYGWNVPPEDDYW | QGIRND | AAS | CLQHNTYPPFF | 1 |
| MS37_p18w62 | GYTFTAYY | INPYSGGT | CAAGPAPASSTWPSNWFDPW | QSVNNY | DAS | CQQRSNWPPFTWTF | 2 |
| MS37_p18w8 | GGPFSGYY | INQSGST | CASSYIFIGPPARAMGDPQWR | QSVGSNF | GAS | CQQCGTSPWTF | 1 |
| | | | HRRGRFDFW | | | | |
| MS37_p19w112 | GIIFTNYW | INIDGSDT | CVRVYYDFWSAPRGMDVW | QSLLHRNGYNY | LSS | CMQALQTPPTF | 1 |
| MS37_p19w119 | GGSFSGYY | ISHSGNT | CARGTLTAEYYFDYW | QRVSDY | DTS | CQQRSGWPTF | 9 |
| MS37_p19w143 | GYSFTSYD | MNPNSGNT | CARGGGYSGYGGDYFDYW | NIGSKN | RDT | CQVWDSSTGVF | 1 |
| MS37_p19w15 | GLPFTNYA | ISRIGDNT | CVSVSSTIFGVVFPSIFESW | NIGSKS | DDS | CQVWDTSSEHYVF | 2 |
| MS37_p19w35 | GFSLGSSGVG | IYWNDVK | CAHERPYCTRMSCSDYYGMDV | SSNIGAGSD | GDN | CQSSDSSLSDSKVF | 1 |
| | | | W | | | | |
| MS37_p19w37 | GGTFSRYG | IIPTVGNT | CARDPIRPSSDDFWSGREDRY | SPNIGNNY | RNN | CAAWDDSLSAWVF | 1 |
| | | | YYFYYGMDVW | | | | |
| MS37_p20w130 | GFTFSSHW | INTDGSRT | CARDAKEARVFDFW | QTISVY | TAS | CQQTYSTPWTF | 1 |
| MS37_p20w145 | GGSFSGYY | INDSGGP | CARLIHGDLYYGMDVW | QGISSY | AAS | CQQFNSYPWTF | 1 |
| MS37_p20w159 | GYTFTAYY | INPYSGGT | CAAGPAPASSTWPSNWFDPW | QRVSDY | DTS | CQQRSGWPTF | 1 |
| MS37_p20w69 | GFNFKTHA | ISGSGSRT | CARRRYDILTGYLHFYAMDVW | QSISNW | KAS | CQQYNSYWTF | 1 |
| MS37_p20w81 | GYSFSNYW | IYPGDSDT | CARHRDYYYYGMDVW | SGSIASNY | EDN | CQSYDSSTLTVF | 1 |
| MS37_p21w13 | GFSLSNAAMG | IFSNDGK | CARRMAAPGQGRVYFDYW | SSNIGNNY | DNN | CGTWDSSLSAWVF | 1 |
| MS37_p21w130 | GYTFTGHY | INPNSGGT | CARPDDGNSVFDYW | SGHSRYT | LYSDGSH | CQTWGTGIQGWVF | 1 |
| MS37_p21w136 | GFTFSSYA | ITGSGDST | CAKDRRFDYYDSSGYYYHDYW | QSISTY | AAS | CQQSYSTPRLTF | 1 |
| MS37_p21w14 | GFSFNNHG | ISSDGNDK | CATPATPRPLVYTSGWYYLDY | SSDVGGYNY | GVN | CSSYAGSNSLVF | 1 |
| | | | W | | | | |
| MS37_p21w2 | GFPFTDYA | ISRIGDNT | CVSVSTSIFGVVFPSIFESW | NIGSKS | DDS | CQVWDSSSEHFVF | 1 |
| MS37_p21w26 | GFSLRNAAMG | IFSNDGK | CARRMAAPGQGRVYFDYW | NIGSKS | DDS | CQVWDTGSEHYVF | 1 |
| MS37_p21w47 | GFRFSNFG | IAYDGTNR | CAKSSRWYDSYYYGMDVW | TSDVGTYNR | EVS | CSSYTRSSTLVF | 2 |
| MS37_p21w6 | GYTFTGFY | INPNSGGT | CARTYDFWSGYFTPPGYW | SSNIGSNY | RNN | CAAWDDSLSGPVF | 1 |
| MS37_p21w82 | GFSFSSYA | VSFDGSSQ | CAKRGPQSGSYFEYW | SSDVGNYDF | EVT | CCSYAGPPTLYVF | 1 |
| MS37_p21w92 | GFAFTNYA | ISRVGDNT | CVSVSSTIFGVVFPSIFESW | NIGSKS | DDS | CQVWDTGSEHYVF | 3 |
| MS37_p21w97 | GFTFSDYF | ISGTNGYT | CVCQRFSTYYFDSW | SSNIGANYD | GNS | CQSYDSSLSIYVF | 1 |
| MS37_p22w100 | GFTFSSYA | ISGSGGST | CAKDSYCSSTTCYMDYW | QSVSSY | DAS | CQHRSNWPPWTF | 1 |
| MS37_p22w106 | GFSLSDPTMG | IFSNDEK | CARSGFCSSTSCLNLDFW | QDIGVW | EAS | CQQYNTYPWTF | 1 |
| MS37_p22w112 | GGSFRGYY | INNWGNT | CVRGGYWQFDFW | TGAVTSGYY | TTN | CLLYHGDAQLWVF | 2 |
| MS37_p22w153 | GDSVSSNSAA | TYYRSKWY | CARGATRAYYFDYW | QTISSW | KAS | CQQYNSYSVTF | 1 |
| | | N | | | | | |
| MS37_p22w158 | GFSLSDPTMG | IFSNDEK | CARSGFCSSTSCLNLDFW | SLRTYY | GKN | CNSRDSSANHVVF | 1 |
| MS37_p22w172 | GFSLGSSGVG | IYWNDVK | CAHERPYCTRMRCSDYYGMDV | SSNIGAGSD | GDN | CQSSDSSLSGSKVF | 1 |
| | | | W | | | | |
| MS37_p22w2 | GFPFTNYA | ISRIGDNT | CVSVSSTIFGVVFPSIFESW | NIGSKS | DDS | CQVWDTGSEHYVF | 2 |
| MS37_p22w44 | GYSFTGYY | INPHTGGK | CARGARNYGPGYNWFDPW | QSLVYSDGNTY | KVS | CMQGTHWRF | 1 |
| MS37_p22w81 | GFSLNDPTVG | IFSNDEK | CARSGFCFSTHCLNLDFW | QNIGVW | EAS | CHQYNTYPWTF | 1 |
| MS37_p23w10 | GYTFTGYY | INLNSGGT | CARLGLRGGFYPYYFDYW | SGSVSTSYY | NTN | CVLYMSSGFWVF | 1 |
| MS37_p23w126 | GFTFSSYG | ISYDGSNK | CAKDLGAVAGGGVYFYYGMDV | SSDVGSYNL | EGS | CCSYAGSSWVF | 1 |
| | | | W | | | | |
| MS37_p23w143 | GFPFTNYA | ISRFGDNT | CVSVSSTIFGVVFPSIFESW | NIGSKS | DDS | CQVWDTSSEHFVF | 1 |
| MS37_p23w145 | GFTFSTYS | ISPSGTNI | CARSGPFPGRVVRWGYFDYW | QSVSGH | DAS | CQQRSDWPPTF | 2 |
| MS37_p23w158 | GYSFTSFW | IYPGDSDT | CARLETVTTNFDFW | SSDVGSYNL | EGS | CCSYAGSSWVF | 1 |
| MS37_p23w163 | GGPMTRHY | IYTRGTT | CARDAYSSSFWFFDLW | QSVRSN | GAS | CQQYNNWPSPWTF | 1 |
| MS37_p23w167 | GYTFTGHY | INPNSGDT | CARPDDGNSVFDSW | SGHSRYT | LYSDGSH | CQTWGTGTQGWVF | 1 |
| MS37_p23w168 | GLTFSKAW | IKSKSDGG | CTTGGQLRRPYW | KLGDKY | QDT | CQAWDVNTEVF | 1 |
| | | TT | | | | | |
| MS37_p23w17 | GFPFSDYA | ISRIGDNT | CVSVSTTIFGVIFPSIFESW | NIGSKS | DDS | CQVWDTSSEHYVF | 3 |
| MS37_p23w171 | GYSFTSFW | IYPGDSDT | CARLETVTTNFDFW | SGSIASNY | EDN | CQSYDSSNLLF | 1 |
| MS37_p23w175 | GYTFTNYF | VNPRRGTA | CAKGGTYHDYWSGYSDAFDMW | TGTVTIGHY | DAN | CLLSYSGADWVF | 1 |
| MS37_p23w176 | GFPFTDYA | ISRIGDNT | CVSVSTTIFGVVFPSIFESW | NIGSKS | DDS | CQVWDTGSEHYVF | 1 |
| MS37_p23w21 | GYTFTSYG | ISAYNGNT | CARGIRGWNDGTDYW | QGIRND | AAS | CLQHNSYPPTF | 1 |
| MS37_p23w24 | GGSFSGYY | INHSGST | CARANRRYSYGYNYHYYGMDV | ALPKKY | EDS | CYSTDSSGNPRGF | 1 |
| | | | W | | | | |
| MS37_p23w3 | GLTVSSTH | IHSDGGT | CVNFGAITSRPW | TGAVTTGHY | DTT | CSLYYSGGVCVF | 1 |
| MS37_p23w30 | GGSFSGYY | INQSGRT | CVHTERNSYDSSGYRGRFDFW | QSVSSSY | GTS | CQQYGTSPWTF | 2 |
| MS37_p23w58 | GGSISDSSYY | IYYSGST | CARMSIDENFDYW | ALPKQY | KDS | CQSVDNSGIYVF | 1 |
| MS37_p23w82 | GGSFSGYY | INHNENT | CARDFHRPDHHCSIGSCYGFD | QSISTY | AAS | CQHSYIAPYTF | 1 |
| | | | VW | | | | |
| MS37_p23w83 | DFSLGSSGVG | IYWNDVK | CAHERPYCTRMSCSDYYGMDV | SSNIGAGSD | GDN | CQSSDSSLSGSKVF | 2 |
| | | | W | | | | |
| MS37_p23w92 | GFPFDTYS | ISSSSRYI | CAKDWGPDLWFGDGSRQFYGM | QSISNNY | GAS | CQQYGSSPRTF | 1 |
| | | | DAW | | | | |
| MS37_p24w10 | GFAFRDYG | LHAYTDTI | CARDKIGSFCIDHW | DSDVAAYNH | GVT | CTSFTTFTTWVF | 1 |
| MS37_p24w108 | GFTFSKYA | ISGSGDTT | CVKGLNYVWGIYRGEYYSYGM | QSLLTSNGNNY | LGS | CMHALQTPGF | 1 |
| | | | DVW | | | | |
| MS37_p24w127 | GFSLGSSGVG | IYWNDVK | CAHERSYCTRMSCSDYYGMDV | SSNIGAGSD | GDN | CQSSDSSLSGSKVF | 1 |
| | | | W | | | | |
| MS37_p24w130 | GESFRGYS | IDHSGST | CARWDGGYRHGSDTYVYYGLD | QSVGSD | DIS | CQQYNNWPPWTF | 1 |
| | | | VW | | | | |
| MS37_p24w145 | GGSISSYY | ISYSGST | CARQKSYYDRGGYWLEGDRSL | QIVSFSF | GAS | CQQYGRSPSTF | 2 |
| | | | AFEYW | | | | |
| MS37_p24w169 | GFPFSNYA | ISLIGDKT | CVSVSSTIFGVVFPSIFESW | NIGSKS | DDS | GQVWDTSSEHYVF | 1 |
| MS37_p24w17 | GYTFTSYG | ISAYNGNT | CASQQSYNWNDVALDCW | QGIRND | AAS | CLQHNSYPPTF | 2 |
| MS37_p24w178 | GFPFTNYA | ISRFGDNT | CVSVSSTIFGVVFPSIFESW | KIGSKS | DDS | CQVWDSSSEHYVF | 2 |
| MS37_p24w18 | GGSFSGYY | INHSGST | CARANRRYSYGYNYHYYGMDV | ALPKKS | EGS | CYSTDSSGNPCGF | 1 |
| | | | W | | | | |
| MS37_p24w180 | GYTFTDCY | INPNSGGT | CVREGLDYSGSFQFDFW | QSVNNY | DAS | CHQRSGWLTF | 1 |
| MS37_p24w20 | GFPFTNSA | ISRFGDNT | CVSVSSTIFGVVFPSIFESW | NIVSKS | DDS | CQVWDTSSEHFVF | 1 |
| MS37_p24w26 | EYTFIDYY | INPKSGDT | CARDSRYCVGGTCKYLKRW | QSVDYNY | GVS | CQQYFGSSLTF | 1 |
| MS37_p24w39 | TGSITTDS | ISGSGRT | CARDRHDWYFDLW | RSDIGGYNS | DVS | CVSYSSGPSPWVF | 1 |
| MS37_p24w48 | GDSISSFH | APNNGDT | CVVYYYGRGGQGFW | QNINTW | MAS | CQQFRTFIWTF | 1 |
| MS37_p24w6 | GYTFTSYG | ISAYNGNT | CASQQSYNWNDVALDCW | QAISSY | VAS | CQQFYSFPVTF | 1 |
| MS37_p24w81 | GYTFTAYY | INPNSGDT | CARDSYYDYVWGSYRLLYGMD | QSLVYSDGSTY | KVS | CMQGTHWPPLF | 1 |
| | | | VW | | | | |
| MS37_p25w10 | GFPFTDYA | ISRIGDNT | CVSVSTTIFGVVFPSIFESW | NIGSKS | DDT | CQVWDTSSEHYVF | 1 |
| MS37_p25w100 | GYTFSSYG | ISAYSGNT | CARGSGYSAVAEFDPW | QGIRND | TAS | CLQHNSYPPTF | 1 |
| MS37_p25w114 | GFPFTDYA | ISRIGDNT | CVSVSTTIFGVVFPSIFESW | NIGSKS | DDS | CQVWDTSSEHYVF | 1 |
| MS37_p25w129 | GFSVSTNY | IYSGGTT | CAKDLAHSDFWSGYLDTW | QSLGKY | DAS | CQQRSNWPLYSF | 1 |
| MS37_p25w14 | GFTFSNYG | ISYDGSNK | CAKLWGGYGDYIGRGEGFDFW | QSISSW | KAS | CQQYDSYLLTF | 2 |
| MS37_p25w167 | GFSLGSSGVG | IYWNDFK | CAHERPFCTRMRCSDYYGMDV | SSNIGAGSD | GDN | CQSSDSSLSGSKVF | 1 |
| | | | W | | | | |
| MS37_p25w177 | GFTFSSYS | ISSSSSTI | CARAHTHCTNGVCSDAFDIW | QGIRND | AAS | CLQDYSYPLTF | 1 |
| MS37_p25w35 | GYTFTSYG | ISAYNGNT | CARGSGYSAVAEFDPW | QGIRND | TAS | CLQHNSYPPTF | 1 |
| MS37_p25w48 | GFPFSNYA | ISLIGDNT | CVSVSSTIFGVVFPSIFESW | NIGSKS | DDS | CQVWDSSSDHPVVF | 1 |
| MS37_p25w57 | GFPFSDYA | ISRIGDNT | CVSVSTTIFGVIFPSIFESW | NIGSKS | DDS | CQVWDSSSDHPVVF | 1 |
| MS37_p25w59 | GFSFSSHG | ISSSSSTI | CARRLWITFGGVIANDYW | QSVGSN | GAS | CQQYNNWPPITF | 1 |
| MS37_p25w8 | GVSITRSTDY | TYHIGST | CARHLSSGWLKLGFDYW | QGIRTW | AAS | CQQTNSFPITF | 1 |
| MS37_p25w81 | GFPFSNYA | ISRFGDNT | CVSVASTIFGVVFPSIFESW | NIGSKS | DDS | CQVWDSSSDHPVVF | 1 |
| MS37_p25w83 | GFTVSSNY | IYSGGST | CAREKAVAGKGGYYYYGMDVW | QRVSSSY | GAS | CQQYGGSPWTF | 1 |
| MS37_p25w89 | GGSFSGYY | INDSGGP | CARLIYGDLYYGMDVW | QGISSY | SAS | CQQFNSYPWTF | 1 |
| MS39_p24w100 | GGSINSYF | IYYSGST | CAASPPRGDIVIVPVAAFDFW | QSVSSD | GAS | CQQYNNWPRTF | 1 |
| MS39_p24w106 | GDSIASSSW | IYHSGHT | CARVKEDIVVGPAGKDYYYYM | QDINNY | DAS | CQQYDDLPLFTF | 1 |
| | | | DVW | | | | |
| MS39_p24w107 | GGSISASSYY | IYYDGST | CARPTPGLYDSWTGFPNGDTC | QSVSNSY | GAS | CQQYGSSPLTF | 1 |
| | | | CYMDVW | | | | |
| MS39_p24w108 | GGSISNYY | IRYSGST | CARTSEYDFWSGYYGFGPW | QSLLHSNGYNY | MGS | CMQAVQTPLTF | 4 |
| MS39_p24w109 | GFTFTDYW | VKEDGSEQ | CARRRQISSSIYFDYW | QGIRND | AIS | CLQDYAYPYTF | 16 |
| MS39_p24w111 | GITFTNAW | IMSENDGG | CTTQKTPYSNPVYFDYW | QGIANF | AAS | CQQANSFPPRAAYTF | 4 |
| | | AI | | | | | |
| MS39_p24w112 | GFSLSTSGMR | IDWNDDK | CARSGFCSGGSCYANWFDSW | QFVSSNY | DTS | CQQYATSPLTF | 1 |
| MS39_p24w114 | AFTFSNYW | IREDGSER | CARVVGLDYGDYGDRRLGEYY | QSIDTW | KAS | CQQYNSYLLTF | 3 |
| | | | FDYW | | | | |
| MS39_p24w115 | GSTFRNSW | IKEDGNEK | CARDGTMTIFGVVYLGWFDTW | QNILYNSNNKNY | WAS | CQQYYKTPRTF | 1 |
| MS39_p24w122 | GFTFSNYA | ISGTGGST | CAKDATDFRSGPRPGFYYYYM | QEISNY | DAS | CQKYNSALRTF | 4 |
| | | | DVW | | | | |
| MS39_p24w130 | GGSISRSNYF | IYYSGGT | CARHFDGGYYYMDFW | QDISSY | AAS | CQHYHGYPITF | 1 |
| MS39_p24w136 | GGSISSNNYY | VFYSGST | CARAQEWLELDGFDMW | QGIRND | GAS | CLQHNSYPYTF | 19 |
| MS39_p24w143 | GGSISSYY | INYSGNT | CARVTSGYSTMWKWGMGEPAT | QSVSSY | DAS | CQHRSNWPLALTF | 1 |
| | | | YYYYMDVW | | | | |
| MS39_p24w144 | GGSISASNYY | IFYSGST | AFVRYSRGHYPPHYYFMDVW | QSINNW | EAS | CQQYSSYFFTF | 2 |
| MS39_p24w145 | GGSISSSSQY | IYESGST | CARLKGNRGYYYMDVW | KIGSKN | RDT | CQVWDSSTEEVF | 1 |
| MS39_p24w146 | GFTFSHYW | IKQDGSET | CAHRIAGAYY | ESLLHDNGFNY | LGS | CMQALLTYTF | 5 |
| MS39_p24w163 | GDSISTYY | ISYSGST | CAREGYSHGYSYYYYYMDVW | QSISNY | AAS | CQQSYNIPRTF | 1 |
| MS39_p2w174 | GFTFSNYW | INQDGSEK | CTRDPPYFDNW | QSLVYSDGRTY | KIS | CMQGSHWPVTF | 2 |
| MS39_p24w168 | GDSISSKNW | IYHSGSV | CARREVGYRLLYGDW | QSVISSY | GTS | CQQYGNSPYTF | 2 |
| MS39_p24w169 | GFSFSTYW | IKGDGSEA | CARHPGSGYYYGWNFDLW | QSVLFNSNNKHY | WAS | CQQYQSIPLTF | 6 |
| MS39_p24w17 | GDSIASSSW | IYHSGHT | CARVKEDIVVGPAGKDYYYYM | QSITTY | AAS | CQQRNTF | 6 |
| | | | DVW | | | | |
| MS39_p24w172 | GDSVSSSNW | IYHSGST | CARGGGYYDSSILIDYW | QGISSF | GAS | CQQLYSYPRTF | 1 |
| MS39_p24w174 | GFTFSNHW | IKRDGSEK | CVRVSLHWARLDYW | QDINNY | DAS | CQQYDDLPLFTF | 7 |
| MS39_p24w177 | GDSVSGGNYY | VYYTGST | CARESYISLDSW | SSNITNNY | KNN | CATWDDSLS | 1 |
| MS39_p24w178 | GESFSDYY | VNRIGNT | CARGRKIVVTVDHWERPNGFD | RSVSSTY | GAS | CQQYGRSPFTF | 1 |
| | | | IW | | | | |
| MS39_p24w180 | GGSISSSSYY | IYYSGST | CARQYLGSSSSGDYW | QSVSSY | DAS | CQQRSNWPPSTTF | 1 |
| MS39_p24w187 | GGSISGSSYY | IYYLGST | CARHEVADMVIVAAALDVW | QSISSW | KAS | CQQYNSYGLTF | 1 |
| MS39_p24w2 | GFTLSSYE | IDSSSDTM | CARDPYLELQWRAFDIW | QGIGDW | AAS | CQQGHTFPPLTF | 5 |
| MS39_p24w24 | GFSLNTAGMC | VDWDGDT | CVRTTVPAAIEGAVRFYYYYL | QDIANY | DGS | CQQYDNVPLTF | 1 |
| | | | DVW | | | | |
| MS39_p24w25 | DGSISSSSYF | ISYSGST | CARQHHGDHYYYYMDVW | QSVSSN | GAS | CQQYNNWLYTF | 1 |
| MS39_p24w26 | GFIFSNYA | ISVGGTST | CARANKYVGSWYFFDYW | ESVGTN | DAS | CQQYDSWPPWAF | 1 |
| MS39_p24w30 | GGSVRSGDYY | VYYSGNT | CARDRIAVTATPGLLDYW | QSISSW | KAS | CQQYNSYPWTF | 1 |
| MS39_p24w44 | LNTDGMR | IDWDDDK | CARNNYYDNSGYSYWYFDLW | QSINNY | ATF | CQQSYTNPFTF | 1 |
| MS39_p24w48 | GGSISSNSYF | MYYSGST | CARHKEVAIATDYYYYMDVW | QSIGTY | AAS | CQQSYSTLSTF | 1 |
| MS39_p24w5 | GYTFTFYY | INPSDGST | CARDSGITFGGLPLNYFDHW | QSISSTN | SIS | CQQYGNSLRTF | 4 |
| MS39_p24w6 | GFSLSTSGVS | IYWDDDK | CAHTNSPINGYYFFDYW | QSVSSN | GAS | CQQYDNWPLYTF | 1 |
| MS39_p24w69 | GGSIGSYY | IYHVGST | CARGPPLTTPTSWSFFFDIW | QIISSW | KAS | CQHYNSYPWTF | 1 |
| MS39_p24w8 | GGSISNYY | IYYTGTT | CAREGSGGYYNWFDPW | QSVLYSSNNMNY | WAS | CQQYYSAPYTF | 4 |
| MS39_p24w81 | GGSFSGYY | INHSGST | CARPGNYLRRWNSGTYYHFDF | QSVLYSSDNKNY | WAS | CQQYYTTPTF | 1 |
| | | | W | | | | |
| MS39_p24w82 | SGSFSDHT | IDYSGST | CARGSRIGGVLAWGPTYNYCY | QSVGSN | DAS | CQQRTNWPPRTLTF | 1 |
| | | | MDVW | | | | |
| MS39_p2w100 | GFSFSSYW | IKQDGSEK | CARYYTRTIYDHDAFDIW | QGISSY | AAS | CQQLNSYPLTF | 1 |
| MS39_p2w101 | GDSISSSNW | IYHSGST | CARSPYDILTGYTDPSDIW | QFVSRY | DAS | CHQRSNWPPLTF | 1 |
| MS39_p2w109 | GGSFSGYY | INPDGST | CAREPGHYYMDVW | QSVLYSSNNKNY | WAS | CQQYYSTPRTF | 1 |
| MS39_p2w114 | GGSISSNNYY | VFYSGST | CARAQEWLELDGFDMW | KIGSKN | RDT | CQVWDSSTEEVF | 1 |
| MS39_p2w119 | GFSLNTERMR | IDWDNHK | CARMGLGWDYFDSW | QSVSNN | GAS | CQQYNDWPRYTF | 3 |
| MS39_p2w122 | GDSISSSNYY | IHYSGST | CARRGGYYFIDYW | QSFSSY | AAS | CQQSYSTLWTF | 1 |
| MS39_p2w127 | GASISGSNW | IYHSEIT | CARDQVRGDFWSGSGDAFDVW | QSVLYSSNNKNY | WAS | CQQYHGSPRTF | 1 |
| MS39_p2w130 | GVSISSSNW | MFLSGST | CARRTMFNYYFDYW | QSVSSY | DAS | CQQRHNWVTF | 2 |
| MS39_p2w144 | GGSVNSINW | IYYSGST | CARDPGGHDYVWGSYYDW | QSLLYSDGYNH | LGS | CMQTLQTPRTF | 1 |
| MS39_p2w153 | GDSISRSNW | IHHSGRT | CARDAGYCRGGSCYDYW | QSVSSN | DAS | CQQYDNWPPLTF | 1 |
| MS39_p2w159 | GGSISSYY | IYHSGST | CAREHRDYYDSSGYYDRW | QSISSW | KAS | CQQYNSYSGTF | 1 |
| MS39_p2w162 | GYTFISYY | INSSGGST | CARGGPTYYYGSGSQLYW | NIGSKS | DNS | CQVWDTIIDPYVVF | 1 |
| MS39_p2w169 | GFTFSSYA | LSGSGGST | CAKAMRWELRSSDYW | SSDVGGYNY | EVS | CSSYTSSSTLGVF | 1 |
| MS39_p2w171 | GGSISTYY | ISYNGNT | CARDVFTGWNHHVGLYNWFDP | QSVSSY | DAF | CQQRSNWPPTF | 1 |
| | | | W | | | | |
| MS39_p2w176 | GYKFTSYG | ISVYNGNT | CARDWYYDSRRDAFDIW | QGLVFSDGNTY | KVS | CMQGTHWPWTF | 1 |
| MS39_p2w177 | EGTFNNYA | IIPIFDTT | CAGGLVTVSGVVIHAGRDWFD | QDISSS | AAS | CQQYYTYPPTF | 1 |
| | | | PW | | | | |
| MS39_p2w178 | GGSISSSSQY | VYETGST | CARLRGNRGYYYMDVW | KIGSKN | RDS | CHVWDSSTEEVF | 1 |
| MS39_p2w180 | GFTFSSNW | INQDGSEK | CARPGYCIGGNCYGRVRLYFQ | QDISNY | GST | CQQYDNLPITF | 1 |
| | | | SW | | | | |
| MS39_p2w184 | GGSINSNNW | IYHDGTA | CARGDNSDRFQISYYFDYW | QSVLYSSNNQNY | WAS | CQQYYSIPNTF | 1 |
| MS39_p2w185 | GFTFGQYA | ISSSDDNR | CAKDWGLFRGGDGYSYYFDYW | NIGSRS | DDT | CQLWDTFSDHFVF | 1 |
| MS39_p2w26 | GFTFSSYA | ISYDGSNK | CARVHDHGDYGWFDPW | QSVSIY | DAS | CQQRRNWPPITF | 1 |
| MS39_p2w36 | GFAFSTSW | IDQAGTVV | CARNRGYQQFDYW | SGDIGRYNY | DVT | CNSFGGLF | 1 |
| MS39_p2w47 | GYNFNDYY | INPDGGGT | CARDEAGRTMSVPFFDYW | QNILYNSNNKNY | WAS | CQQYYTYPYTF | 1 |
| MS39_p2w48 | GFSLSTSGLR | IDWDDDK | CARDYYFDSSGYRFDYW | QDISNY | DAS | CQQFDHLYTF | 1 |
| MS39_p2w62 | GGSIVSYY | IYYSGST | CARGGGGFHDPSPNYSPDYW | QGIRND | AAS | CLQYNSYPPTF | 3 |
| MS39_p2w69 | GFTLTNYA | ISGPTGST | CAKGRDPVENVVIFPFDCW | QDINMW | KAS | CQQYYSLHSF | 1 |
| MS39_p2w7 | GYTFISYG | ISAYNGYT | CARDGLTYCGGECFFAYW | QSVSSSY | GAS | CQQYVSSPLTF | 1 |
| MS39_p2w89 | GDTFSNYA | IIPIYDTV | CARDGWRYDGRGHYELNYYYM | QTVSSN | GAS | CQQYKNWPPTF | 1 |
| | | | NVW | | | | |
| MS39_p3w100 | GFSFSNYV | LSAGGGAT | CVKNQGIYGSGSYPADTFHIW | QNIFHSSNNKIY | WAS | CQQYFSTPFTF | 1 |
| MS39_p3w101 | GGSISSSSYY | VYYIGNS | CARGGYYYYYMDVW | QSVNSDY | GAS | CQQYFSSPHTF | 3 |
| MS39_p3w118 | GFSFSTPGMA | INWDDDE | CAHRGFYQPQYFDTSAYYYW | SSNIARHY | NNN | CAVWDDSLSGWVF | 1 |
| MS39_p3w119 | GFSLTTPGLT | IFGDGET | CAHSHAIGDNYLSYFDFW | QSVLYDPNKKNY | WAS | CQQYYSGPITF | 1 |
| MS39_p3w126 | GFTFSNYA | ISGGDHST | CAKGFGDYRYYYYMDVW | ESLVHSDGNTY | KIS | CMQAKQFPLTF | 1 |
| MS39_p3w127 | GD | ISPFGTS | CAARPPFLGYRDSTICHVGSA | QSLLHFNGYSY | LGS | CMQVLQTPRTF | 1 |
| | | | P | | | | |
| MS39_p3w144 | GFTFTSYW | IKEDGNEK | CARDLTVFGVLDYYYMDVW | QSLLHSNAYNY | LTS | CMQALQTPHTF | 2 |
| MS39_p3w145 | GFSLNTSGVG | IYWNDDE | CAHNGGYDFRSGYYWAHWFDP | QSLSSRY | GAS | CQQYDSSFTF | 1 |
| | | | W | | | | |
| MS39_p3w163 | HGSISTYH | IYYIGST | CARGPPTGEWSYYFDNW | QDVSSW | KAS | CQHYNSYPWTF | 1 |
| MS39_p3w17 | GFTFSSYW | IKQDGSEK | CARDRWSGSFGGAPLDSW | QDVTNY | EAS | CQQYDILPPTF | 1 |
| MS39_p3w175 | GDAVSSGSYY | VYHTGST | CARGVGLAGAGTSFDYW | QSVLYSSNNINY | WAS | CQQYYTTPATF | 1 |
| MS39_p3w184 | GGSISSTNYY | VYHSGST | CARRDDFWSGYYDYW | RTINTY | AAS | CHQTYSPPQTF | 1 |
| MS39_p3w25 | GGSVSSGTYW | IYYSGST | CARLGVGELSLLGFGAFDIW | QSVNSN | GAS | CQQYNTWPLAF | 1 |
| MS39_p3w3 | GFTFSRYW | IKEDGSEK | CAREANFWSGYFDYW | QSIDTY | AAS | CQQSYSAPWTF | 1 |
| MS39_p3w30 | GGSFTGYY | IHHRGRT | CARAEDSEIFGVVANTWFDPW | QSVINS | RAS | CQQYNNWPTF | 1 |
| MS39_p3w48 | GDSISSSTYY | IFYTGNT | CASRRITIFGVGELIDWVDPW | SSNIARNY | NNN | CAVWDDSLSGWVF | 1 |
| MS39_p3w5 | GFTFNDYW | IKQDGSEK | CARRRESGSSIYFDYW | RDIRND | AAS | CLQDYSYPYTF | 1 |
| MS39_p3w51 | GDSVSSGTYW | IYYSGST | CARLGVGELSLLGFGTFDIW | QSVSSN | GAS | CQQYNTWPLAF | 1 |
| MS39_p3w8 | GGSISSSSYY | IYNSGNT | CTRPPWAFWSEYYQMGDYYYM | QSITSN | GAS | CQQYNNWPFTF | 1 |
| | | | DVW | | | | |
| MS39_p3w94 | GASISSTDW | VSRSGTS | CAREPYDSWLGYIDVW | QSVLYSPNSKNY | WAS | CQQYFSSPYTF | 1 |
| MS39_p3w97 | GFTFNTYE | ITSSGSTI | CARAAYLHFWSDYPRGWFDPW | QSLLHSNGKTY | EVS | CMQSTQLLGTF | 1 |
| MS9_p11w10 | GFSLSNARMA | IFSNDEK | CARIRVGYNYGPDAFDIW | RGISSY | ATS | CQQLKSYPLTF | 1 |
| MS9_p11w100 | GFTVRNSY | IYNNGNT | CARVEYGNSSGAFDIW | QSVNNN | DAF | CQQYNYWPIFTF | 2 |
| MS9_p11w109 | GFSLSNTKMG | SFSNDEK | CARKSIAVAGRPFDYW | QSISSY | AAS | CQQSYSTPPWTF | 1 |
| MS9_p11w112 | GFSLSNPRMG | IFSNDEK | CARIRVGYNYSPDAFDIW | RGISNY | AAY | CQQLNSYPLTF | 1 |
| MS9_p11w113 | GGSISSSFYY | IYYSGST | CAREEAAASWLEYW | QSVTSN | GAS | CQQYNNWPPLVTF | 1 |
| MS9_p11w115 | GFSFSNYW | IKEDGSQK | CARVQRAAIGYFQYW | QSISTF | AAS | CQQSYSTPSFTF | 1 |
| MS9_p11w118 | GFSLSTGGVG | IYWSDDQ | CARDGTRLRFLEWSLGPTFDI | QSISRW | KAS | CQQYNSYSSTF | 2 |
| | | | W | | | | |
| MS9_p11w13 | GGSISSYDW | ISHSVST | CARYITMVRGVFIQGRGWFDS | QSIGTY | VAS | CQQNYIIRTF | 2 |
| | | | W | | | | |
| MS9_p11w130 | GGYITAYY | VHYTGST | CARDRYCSDNSCPQGRYHFYY | QSISSY | AAS | CQQAYSNSRTF | 1 |
| | | | MDVW | | | | |
| MS9_p11w133 | GFTVSTNY | LYTTGQT | CARVEYGRSSGAFDYW | QSVNSN | DTS | CQQYNYWPKFTF | 1 |
| MS9_p11w14 | GFSLSTSGVG | IYWNDDK | CTHRRRYFDYW | QSVSSY | DAS | CQQRSNWPWTF | 1 |
| MS9_p11w143 | GFTFSNYW | IKQDGGEK | CAKTHSMGGIQWRAQGFDYW | QDISNY | DAS | CQQYDNLPVTF | 2 |
| MS9_p11w144 | GFTFSMYW | IKQDGSEK | CDRPSRPLLPRSAFDIW | QSVSSNY | GAS | CQQYSSSPPPLTF | 1 |
| MS9_p11w145 | GGSISRDY | IYYIGST | CARQPFGGSGWFGWFDTW | QSIDNW | TAS | CQHYNSYPYIF | 1 |
| MS9_p11w159 | NYW | INEDGSEE | CARHRRDFVVVTAGSDLSYNQ | QSINSDY | GAS | CQVYGTSPTFTF | 1 |
| | | | YYYMDVW | | | | |
| MS9_p11w163 | GGSISGYY | IYYSGST | CARSSHDFGDYEAEYFQHW | QSLSSF | GAS | CQQTYSIPWTF | 1 |
| MS9_p11w169 | GDSISGYF | IYYSGIT | CARHVNMAVAGGNWFDPW | HSISSY | GAS | CQQTYSSPPTF | 1 |
| MS9_p11w175 | GGSINTGHYY | IYNSGST | CARAHWRWFAAGPMDVW | QDVNSY | DAS | CQQYDTLPLTF | 12 |
| MS9_p11w176 | GFSLSTSGVG | IYWNDDK | CAHSPPPNDFWSGYYLGGGGD | QSVSSD | GAS | CQQYDHWPTTF | 1 |
| | | | WYFDLW | | | | |
| MS9_p11w20 | RFMFSSYW | IKQDGSEK | CARDRAGFWSAYFDYW | QSISSN | GAF | CQEYNNWPPWTF | 4 |
| MS9_p11w21 | GGSIRRSSYY | IYFSGST | CARDLRGYNYGLDSW | QSLEHNDGNTY | KVS | CMQGTQWPLYTF | 1 |
| MS9_p11w3 | GGSISKSTYY | IYYSGNT | CARDSRAILNSGGLDSW | QSVNSN | GAS | CQHYNDWPLRDTF | 2 |
| MS9_p11w35 | GASI | IYYSGAT | CAKGGNYYDTGSFLWGIRPL | QSISTS | AAS | CQQSYITPRTF | 1 |
| MS9_p11w37 | GFTFSNYG | IWFDESNK | CAREGGYCNHGNCYGMAWFDS | SSDVGSYNL | EVN | CCSYTGRSSWVF | 2 |
| | | | W | | | | |
| MS9_p11w47 | GGSISSSSFF | INYSGTT | CARHWRYSISGSGNWFGPW | SSDVGGYPY | DVT | CCSYAGSSTFVF | 1 |
| MS9_p11w5 | GFSLSTSGMC | IDWDDDK | CARISKMVYYGSESYYFDYW | QSITNY | GAS | CQQSYSIPWTF | 1 |
| MS9_p11w51 | GFTFRSYA | ISGGGGSI | CARYPSGWRLNDAFDIW | QSLVHSDGNTY | KVS | CMQGTHWPPVYTF | 1 |
| MS9_p11w55 | GFSLSNPRMG | IFSNDEK | CARTEVGYGVVIVKPFDIW | QSVSSSY | GAS | CHQYGSSPLTF | 2 |
| MS9_p11w69 | GFSLSNARMG | IFSNDEK | CARTIDTSYYDFWSGSTTGWY | QSVLYSSNKQNY | WAS | CQQYFNSPVTF | 1 |
| | | | FDLW | | | | |
| MS9_p11w7 | GFSLSSSGLC | IDWNDDK | CARILVRGGFDYW | QGIGSW | AAS | CQQANSFPPWTF | 1 |
| MS9_p11w8 | GGSISGYY | IDDSTYT | CGRHQYSWFDLW | QSIDRW | MAS | CQQYDTYPWTF | 1 |
| MS9_p11w81 | GVSISSGRYY | IYYDGST | CARDGRGNSGYDWGYYFDYW | ETISNY | DVS | CQQSYGIPRTF | 1 |
| MS9_p12w106 | GFSFTTSGVG | IYWNDEN | CAHAQRRGRNNWYGSSFDMW | QDISDF | GAS | CQQYDHFLTF | 1 |
| MS9_p12w108 | GFSLSNARMG | IFSNDEK | CARIITPSFYDFWNGYLYYFD | QSFSNN | GVS | CQQYSNWPPTF | 1 |
| | | | YW | | | | |
| MS9_p12w111 | GGSVSNYY | IYTSGNT | CARRGSYTLWSGYPVFDSW | QSLLYSNGYNY | LGS | CMQAVQSPPTF | 1 |
| MS9_p12w112 | GYSFTDYW | IYPGDSET | CARGQLERRHGLYYDILTGSR | KLGDKY | QDT | CQAWDSSTVVF | 1 |
| | | | SLRPKHQNWIDPW | | | | |
| MS9_p12w113 | GGSISSSGHY | IYYSGST | CAKPAMGSIRGWFDPW | QSVYSN | GAS | CQQYNNWPGTF | 1 |
| MS9_p12w114 | GFSFRSYV | ISYDGRNK | CASAQWGCSSTSCYTLDSW | ILRSYY | GKN | CNSRDSNDNHLTVF | 1 |
| MS9_p12w115 | GGSIGSSSDY | ISYGGVT | CASHLPYVYFYYYYMDVW | QSVSSY | DSS | CQQRLNWLTF | 1 |
| MS9_p12w122 | GFSLSTSGMC | IDWDDDK | CARMVRAGYSYYMDVW | QSISSC | AAS | CQQANSFPQTF | 1 |
| MS9_p12w127 | GGFISSSSSY | IYYSGTT | CASAPYYDFWSGYYGDGFDIW | QSLVHSDGKTY | KVS | CMQGTHWPLTF | 1 |
| MS9_p12w13 | GFTFNSYA | ISGSGGDT | CAKLRGYTWNADLDYW | QGIRND | AAS | CLQHNSYPPTF | 1 |
| MS9_p12w130 | GFSLSNARMG | IFSNDEK | CARTIDTSYYDFWSGSTTGWY | QSVLYSSNNKNY | WAS | CQQYFNSPVTF | 1 |
| | | | FDLW | | | | |
| MS9_p12w133 | GFSLSISGVG | IYWNDDK | CAHRPYNFWSAYYFDYW | QSLLHSDGYTY | EVS | CMQDAQDPPFTF | 1 |
| MS9_p12w135 | GFTFNSYA | ISGSGGDT | CAKLRGYTWNADLDYW | SSNIGITT | SNN | CAAWDDRLNGPVF | 1 |
| MS9_p12w14 | GGSITRYY | FYYSGNT | CARHLGVMYAFHIW | QDIGNY | DAS | CQQYDNFPPTF | 2 |
| MS9_p12w145 | GGSISDTTYY | VISSGHT | CARAPYYNFWSGYWFDYW | QGITNW | GAS | CQQANSFLGTF | 1 |
| MS9_p14w183 | GGSISSSDYY | VYYSANT | CGRVRGTVYGVLIKGRPYYYM | QTISKW | RAS | CQHYDNYWWTF | 3 |
| | | | DVW | | | | |
| MS9_p12w153 | GGSISSSSYY | IYYSGST | CARGDIDTGVDSW | QSLVYSDGNTY | KVS | CMQGTHWPPTF | 1 |
| MS9_p12w167 | GGSITTNHYY | ISYSGST | CARETSVMEGFYYYYNMDVW | QSIGRKY | GAS | CQHYGSSPRYTF | 1 |
| MS9_p12w171 | GFSLSNARMA | IVSNDEK | CARLLIGYQFLPYYFDNW | QGISNY | GAS | CQKYNNALLTF | 1 |
| MS9_p12w175 | GFTFSNYW | IKQDGSEK | CARDVRWEGLPDIVVVPAATH | QSVLYSSNNKNY | WAS | CQQYYSTPRTF | 1 |
| | | | EFHMDVW | | | | |
| MS9_p12w177 | GGSISSSTYY | ICCGGST | CARAFTGHPDYDFWRWFDPW | QSVLYSSNNKNY | WAS | CQQYYSNPITF | 2 |
| MS9_p12w18 | RGSISSSTDY | MFHSGAT | CARGRRGDFWTTSFREGSTRL | QGISNW | EAF | CQQANSFPLTF | 1 |
| | | | EYFDYW | | | | |
| MS9_p12w180 | FTFSSYW | INQDGSEK | CARDATSNYVHYYYYMDIW | QSVLYSSDNKNY | WAS | CQQYYSNPRTF | 1 |
| MS9_p12w183 | GGSINSYY | IYNSGST | CARDVEILVNPGHWFDPW | QSISSY | AAS | CQQSYSSLPFTF | 1 |
| MS9_p12w20 | GFSLSNTRMG | IFSTDEK | CARISNYDFWSGSYDAFDIW | HGIRND | AAS | CLQHNSYPPTF | 1 |
| MS9_p12w22 | GFSLTTGGVG | TYWNDDE | CAHSGERCTGGRCYYFNDAFD | QSLLSRSNNKYF | WAS | CQQYYSTVTF | 1 |
| | | | IW | | | | |
| MS9_p12w30 | GGSINSGDHY | IYFSGTT | CARDMSGYYVDSW | QSILYRSNNKNY | WAS | CHQYYSVPRTF | 3 |
| MS9_p12w35 | GGSISSGGYS | IYLSGRT | CARGLCSGTYCPYFDYW | QDINKY | DAS | CQHFHSLPYTF | 1 |
| MS9_p12w37 | GGSISSSPYY | IYYSGST | CARPNDILTGFYAFDLW | QSISSN | GAS | CQRYNNWPPYTF | 3 |
| MS9_p12w44 | GFTFSSYR | ISSSSSYI | CAREMTLSEMWRRSPQYYYYM | QSIGTH | AAS | CQQSYSFPHTF | 7 |
| | | | DVW | | | | |
| MS9_p12w46 | | IKQDGSEK | CARDVRWEGLPDIVVVPAATR | QSVLYSSNNKNY | WAS | CQQYYTPPRTF | 1 |
| | | | DYYMDVW | | | | |
| MS9_p12w48 | GGSFSGSY | INHSGST | CARQNMPSRSLDSW | QSVLYSSNNENY | WAS | CQQYYTTPPTF | 6 |
| MS9_p12w5 | GFTFSDHW | INQDGGEE | CARLVWRVSDYW | QSLVHSDGDTS | GVS | CMQTIHWPWTF | 1 |
| MS9_p12w51 | GYTFTSYA | ISGSGDNT | CANGALRFLEWLYFDYW | QSVSSN | GAS | CQQYNNWPPWTF | 1 |
| MS9_p12w59 | GFMFSNYC | IKHDGSEK | CARDLRPISFLGVDPNYYYLH | QSVRGSY | GAS | CQQYDSSPQTF | 1 |
| | | | MDVW | | | | |
| MS9_p12w62 | RYTFTKYF | INPYNDNT | CVRDPLGIDLRNITIFFVLL* | QSISSW | KAS | CQQYNSYLTF | 1 |
| | | | LL | | | | |
| MS9_p12w92 | GFSLSTSGMC | IDWDDHK | CARVRLSSSGWYVGFADYW | QSFSSY | DAS | CQQRSNWPLTF | 1 |
| MS9_p13w10 | GFTFSSYA | ISDSGVNT | CAKDGTPGHFWSGYLDYW | QSISSW | KAS | CQQYNTYSRTF | 2 |
| MS9_p13w101 | GDSMTGSLYY | IYYSGST | CARAVTRWGFGDRNTWFDPW | QSVSRN | GAF | CQQYNDWPGTF | 1 |
| MS9_p13w111 | GFSFSSFW | INQDGSEK | CARGGEGGIWSGYNLFDYW | QGISDY | AAS | CLQHNTYPGTF | 1 |
| MS9_p13w112 | GGSISNVY | IYNSAST | CAREATGESMLDYW | QYFSTS | | CQQYYDYPLTF | 2 |
| MS9_p13w113 | GFSFTSSW | IDPSDSST | CARLAIYCDGGGCYSTNYNWF | SSNIGSNT | RNS | CASWDDSVNGPSF | 1 |
| | | | GPW | | | | |
| MS9_p13w114 | GFTFSSYW | IKLDGSEK | CASLSYDFWSGYSPPFDYW | QSVSSRY | GAS | CLQYDPSPPWTF | 2 |
| MS9_p13w115 | GFTFSSYQ | ISSSGTTT | CARTEVSGFGVETFDPW | QSISSY | ATT | CQQSFSTLKTF | 1 |
| MS9_p13w118 | GDSISGYF | IYYSGIT | CARHVNMAVAGGNWFDPW | HSISSY | AAS | CQQTYSSPPTF | 1 |
| MS9_p13w119 | GFTFGDYG | IGSSAYDG | CSRGDYDFWSGYRNLFDYW | SSDVGNYDS | TVN | CWSYTSSATCAIR | 1 |
| | | TT | | | | | |
| MS9_p13w127 | GFSLSTSGVG | IYWDDDK | CAHSSPAAMGGGMDVW | KLGDKY | QDS | CQAWDSSTAWVF | 1 |
| MS9_p13w143 | GGSIGSFY | IYYSGSS | CARGTGWNSGCYWGDCFSPPF | QSVSSSY | DAS | CQQYGNSPPVTF | 1 |
| | | | FDIW | | | | |
| MS9_p13w15 | GGSINTGHYY | IYNSWST | CARAHWRWFAAGPMDVW | QSLEHNDGNTY | KVS | CMQGTQWPLYTF | 1 |
| MS9_p13w158 | GGSISSYY | IYYSGST | CARGPYCSGTNCLGFDYW | QSLLHSDGKTY | EVS | CMQGKHLITF | 1 |
| MS9_p13w167 | GGSIRGSSYY | IYFSGST | CARDLRGYSYGLDHW | QSLEHSDGNTY | KVS | CMQGTHWPLYTF | 2 |
| MS9_p13w168 | GGALGSGNYY | ISYSGST | CARDDIVVVPAAISPTYNWFD | QGIRSW | AAS | CQQAHSFPRTF | 1 |
| | | | PW | | | | |
| MS9_p13w171 | GGSISSGSYY | IYFLGST | CASLTFDYSSPYYFDYW | QVISTW | AAS | CQQANSFSALTF | 1 |
| MS9_p13w178 | GGSISNFY | IYNSATT | CAREATGESMLDYW | QYFSTS | | CQQYYDYPLTF | 1 |
| MS9_p13w18 | GFSLTTRGVG | IYWNDDR | CAHTSNHNRRGYYNGAFDYW | QDISNY | DAS | CQQSDHFLSF | 1 |
| MS9_p13w2 | GFTFSSYA | ISGKGDRT | CARDRPDKYRAWGYQYFYGLD | QSVSSN | GAS | CQQYSNWPPGTF | 1 |
| | | | VW | | | | |
| MS9_p13w26 | GFMFSNYW | IKHDGSEK | CARDLRPISFLGVVPNYYYLH | QSVSGSY | GAS | CQQYESSPQTF | 1 |
| | | | MDVW | | | | |
| MS9_p13w3 | GGSISSHY | FYSSGNT | CARHLGVMYAFNIW | QDIGNY | DAS | CQQYDNYPPTF | 1 |
| MS9_p13w39 | GVSISSNSYY | IYYSGST | CAIQKTVTVPFDYW | QDISKY | AAS | CQQYDIVPGAF | 1 |
| MS9_p13w44 | GFTFSNYW | IKQDGSEN | CARDSSSWYYYYYYMDVW | QSVLYSSNNKNY | WAS | CQQYYTTPRTF | 1 |
| MS9_p13w5 | GASISTSSFY | IHSSGSS | CARALDFDMWTPDYLHRSFDY | QSLRSSY | DAS | CQQYASSPLAF | 1 |
| | | | W | | | | |
| MS9_p13w51 | GFSLSTSGMC | IDWDDDK | CARIQEGAATVVGAFDIW | QDITNY | DAF | CQQYDNLPYTF | 1 |
| MS9_p13w55 | GFSFYTRGVG | IYWDGDE | CARRYSAYDSRGHYHYYALDV | QSLLHSDGNTY | EVY | CLQSKQLLTF | 1 |
| | | | W | | | | |
| MS9_p13w58 | RRSISSYDW | ISHSVST | CARYITMVRGVFIQGRGWFDS | QSIGTY | VAS | CQQNYIIRTF | 1 |
| | | | W | | | | |
| MS9_p13w7 | GFSLSISGVG | IYWNDDK | CAHRPYNFWSAYYFDYW | QDISNY | AAS | CQQYNSYPPTF | 2 |
| MS9_p13w8 | GFTFSSFW | IKQDGSEK | CARDSTLLWFGELEDYFDYW | EIISSY | AAS | CQQSYSTPRTF | 1 |
| MS9_p13w89 | GFTFTNYD | TGTAGDA | CTRHLQ*GFLWSEDGHSATVV | QSINSDY | GAS | CQVYGTSPTFTF | 1 |
| | | | TTTTTLWTS | | | | |
| MS9_p14w107 | GGSISSGAFY | IYYSGTT | CAREGYYSSGSYYNVDAFDIW | QSIRTY | ASS | CQQSYNTPRTRTF | 1 |
| MS9_p14w111 | GGSISSYF | IYYSGST | CARHEHCGSPNCYEVGLFDPW | QSIRSY | AAS | CQQSFSTPYTF | 1 |
| MS9_p14w115 | GFMFSSYW | | CARDLRPISFPGVVPKLLLPP | QSVSGSY | GAS | CQQYDSSPQTF | 1 |
| | | | HGRL | | | | |
| MS9_p14w119 | GDSIRSSGSY | IFYSGNT | CARVVSDGDFWSGYWAYW | QIIGSW | RAS | CQQYNSFPYTF | 1 |
| MS9_p14w122 | EFTVSSNY | IYTGGQT | CARVEYGRSSGAFDVW | QSVNNN | DAS | CQQYNYWPKFTF | 1 |
| MS9_p14w126 | GDSISSGDYY | MYYTGSA | CARGADYYGSGGLHW | QSIGGY | AAS | CQQSYSTPYTF | 1 |
| MS9_p14w129 | GGSINNFY | ISYSGST | CARRAGEYLRLWTRTPQNYYY | EDIRNH | AAS | CLQDYNYPLTF | 1 |
| | | | YYMDVW | | | | |
| MS9_p14w130 | GFTFSTYT | ISSSSDYI | CATLGGGYVRNLYDYW | QSVNSY | DAS | CQQRSNWPLTF | 1 |
| MS9_p14w133 | GFSLSTSGMC | IDWDDDK | CARMVREGYSYYMDVW | QSISSW | AAS | CQQANSFPQTF | 1 |
| MS9_p14w135 | GDSVSSSSYY | LYYTGST | CARGHVVGWLRPFDSW | QGVSSN | GAS | CQHYNNWPPITF | 2 |
| MS9_p14w146 | GFTFSDFW | IKQDGSEK | CARDVRWEGLPNIVVLPVPKQ | QSVLYSSNNKNY | WAS | CQQYYSTPRTF | 1 |
| | | | DFYMDVW | | | | |
| MS9_p14w153 | GVSISTYY | IYYSGST | CASSPQVWLPFDYW | QSVSSN | GAS | CQQYNTWIPYTF | 1 |
| MS9_p14w158 | GGSISSSSYY | LYYSGHT | CARDLSGGDAFDIW | QSISSN | GAS | CQQYNKWPRMYTF | 2 |
| MS9_p14w172 | GGSFSGYN | INHSGST | CAKGVVVLPPASNWFDPW | QSISSN | GAS | CQQYNNWPPERTF | 1 |
| MS9_p14w22 | GLTVSDNY | IYTDGST | CARDFYPFWYFDLW | QSLLHSNGYTH | LAS | CMQALQNMYTF | 2 |
| MS9_p14w30 | GGSISNFY | IYNSATT | CAREATGESMLDYW | QYFTTS | | CQQYYDYPLTF | 1 |
| MS9_p14w39 | GYSFTSYL | ISGYNGNT | CARGGSGWYLTSDYW | TGPVTSASS | RTD | CLLDFDGTRVF | 1 |
| MS9_p14w44 | GGSINSSSYY | IYYSGST | CATSLRPLVRGLFSPRYNWFD | QSVLYSSNNKNY | WAS | CQQYYSSPQTF | 1 |
| | | | PW | | | | |
| MS9_p14w47 | GLTFSTAA | VGPSGTST | CAKEGDFWSGYYSGYFDLW | QSISSQ | DAS | CQQRFNWPLFTF | 1 |
| MS9_p14w6 | GGSISSYY | IYYSGVT | CARHIRGGFHMDVW | QSLLHSNGYNY | LGS | CMQALQTPLTF | 1 |
| MS9_p14w8 | GFSVSNARMG | IFSNDEK | CARVEDFGVIIPGFFDYW | QSISTW | KVS | CQQYHSFSWTF | 1 |
| MS9_p14w92 | GFSLSNARMG | IFSNDEK | CARVEDFGVIIPGFFDYW | QSISTW | KAS | CQQYHSFSWTF | 1 |
| MS9_p14w94 | GFSLSDPRMG | IFSKDEK | CARIRVGYNYSPDAFDLW | HDVRND | AAS | CLQHNSYPLTF | 1 |
| MS9_p14w97 | GGSISSYY | IYYSGNT | CARHPFDFWSGDYTEKGYNWF | QDISNY | DAS | CQQYDNLPITF | 2 |
| | | | DPW | | | | |
| MS9_p15w10 | GGSMNSGRYS | IYQSGTI | CARGFGGNGDSSLSRRRNYGM | QGISNY | AAS | CQQLSSYPWTF | 1 |
| | | | DVW | | | | |
| MS9_p15w100 | GFSLSNVRMG | ILSNDEK | CARTTTVFGVEPW | QGIRND | AAS | CLQDYNYPWTF | 1 |
| MS9_p15w104 | GGSISSSSYY | IYYSGST | CARDGIYNWNDAPHVPVMFDS | QSVSSY | DAS | CQQRSNWPPGTF | 1 |
| | | | W | | | | |
| MS9_p15w113 | GASIRRSNYY | VYSSGST | CARDLRGYNYGLDNW | QSLVYSDGNTF | F | CMQASHCV | 1 |
| MS9_p15w114 | GFNLSNNY | IYSDGST | CARELVVLVGATRANDALHFW | QSVLFSPKNKNY | WAS | CQQYYSPRRTF | 1 |
| MS9_p15w126 | GGSISSSDYY | IYFIGIT | CARVGGSSCGYNWNDCGWVDP | QSVSST | GAS | CQQYKNWPYTF | 1 |
| | | | W | | | | |
| MS9_p15w127 | GFSLYTRGVG | IYWDGDK | CARRYSAYDSRGQYHYYAMDV | WSNITNIN | RNN | CVTGDDNLNSQVF | 1 |
| | | | W | | | | |
| MS9_p15w129 | GGSFNTASHY | IYRGST | CARGLPNYDLLTGSSPYNWFD | QSVSSSY | GAS | CQQYGSSPPGITF | 1 |
| | | | PW | | | | |
| MS9_p15w13 | GFTFSSAW | IKTNTDGG | CTTDFSYTSGWNYW | QSLVYSDGDTY | KVS | CMQGTHWPRYTF | 1 |
| | | TT | | | | | |
| MS9_p15w130 | GFSLSTSGLC | IDGDNDK | CARMGVNRGFLGWGGPSRHPY | SSDVGGYNY | EVS | CSSYAGSNFVVF | 1 |
| | | | YYYGLDVW | | | | |
| MS9_p15w135 | GGSISGTNYY | IYYSGQT | CARVAGDIVGVSDATVWFAPW | QSIARN | AAS | CQQGYNTPPTF | 1 |
| MS9_p15w136 | GFTFSSYA | TINNGATT | CAKGIPWEDIVDAFDIW | QGIRND | SAS | CLQHNRYPPIF | 1 |
| MS9_p15w143 | GFTFSSYW | IKEDGSEK | CARGAFFGLGYNWFDPW | QGIGNY | AAS | CQQLTSYPWTF | 1 |
| MS9_p15w145 | GFTFSNYG | ISYDGSNK | CAKDRYYYESSGYYLFDYW | SGSIASNY | EDK | CQSYDNSNKIF | 1 |
| MS9_p15w164 | GFTFDGYA | ITWNSAII | CARAHSYAAWEW | KLGDKY | QDN | CQAWDTSAAWGVF | 1 |
| MS9_p15w169 | GFTFSNYW | IKEDGSEK | CVREVIIGYQWFDPW | QSVRFN | GAS | CQHYNNWPTF | 1 |
| MS9_p15w18 | NYW | IKEDGTEK | CTRPTAVHQLLYRRLPNWFDS | QSVSSY | AAS | CQQSYSTPLYTF | 1 |
| | | | W | | | | |
| MS9_p15w2 | GGSIISTNSY | IYHSGTT | CARALPTIGYCTGGNCYARWW | QSINSN | GAS | CQQYNNWTPHF | 1 |
| | | | FDPW | | | | |
| MS9_p15w20 | GGSISSSNYY | IYYSGST | CARDGAPLRFLEWLQLHWFDP | QDISNY | AAS | CQKYNSAPLTF | 1 |
| | | | W | | | | |
| MS9_p15w22 | | IRSNAYGA | GVRGSALTT | QSISSW | KAS | CQQYNSYSEYTF | 1 |
| | | TR | | | | | |
| MS9_p15w25 | GGSISSSNYY | IYYSGST | CVRPTIRVGWFDPW | QGISNS | AAS | CQQYYSIVPDTF | 1 |
| MS9_p15w3 | GFTFSSYW | IKSDGSET | CARARIVSLPAAIRWGSDTYY | QTISSF | AAS | CQQSYSNTWTF | 1 |
| | | | YYYMDVW | | | | |
| MS9_p15w43 | GFTFTRYW | IKQDGSEK | CATDLWAGSSSIGDYW | QSVYGNY | GAS | CQQYGSSPWTF | 2 |
| MS9_p15w48 | GGSINTYY | IYYTGST | CARDHRCSSTSCQRDYYYYYM | QSITRF | AAS | CQQSYSTPWTF | 1 |
| | | | DVW | | | | |
| MS9_p15w55 | AGAITNNNYY | IYYSGTT | CARTSGGYLADFDSW | SSDVGSYEN | NVN | CCSCASRATNVY | 1 |
| MS9_p15w59 | GFTFINYA | ISGSGGST | CAKAEIYDFWNAYDAFDMW | QSLVHIDGNTY | KVS | CMQGTHWPPTF | 1 |
| MS9_p15w6 | GDSISSTNW | IFRSGST | CARRLWGGSAFDIW | QSLSSY | DGT | CQQRTNWPPLTF | 1 |
| MS9_p15w7 | GDSISNYY | MYYIRNT | CARDRGGFPKGGGGTLSWGRP | QRIGSSY | GAS | CQQYGNSLWTF | 1 |
| | | | SYYSYGMDVW | | | | |
| MS9_p15w83 | GFSLSTGGVG | IYWNDDE | CARDGTRLRFLEWSLGPTLDI | ESIGRW | RAS | CQQYNTYSSTF | 1 |
| | | | W | | | | |
| MS9_p16w10 | GGSITTYY | FYYSGST | CARHLGVMYAFHIW | QDIGNY | DAS | CQQYDNFPPTF | 1 |
| MS9_p16w104 | GDTFNNFA | IIPVFGTI | CVSGRDFDSTYYYGLDVW | SSNIGAGYD | GNS | CQSYDSSLSGVLF | 1 |
| MS9_p16w106 | GFSLSTSGMC | IDWDDHK | CARVRLSSSGWYVGFADYW | QSVSSY | DAS | CQQRSNWPLTF | 1 |
| MS9_p16w109 | GGSIISINYY | IYYSGTT | CARATPQFQQLWQRYEGWDNW | QTINNY | DVS | CQQRGTWPPYTF | 1 |
| | | | FDPW | | | | |
| MS9_p16w112 | GFSLSTTGMC | IDWDDAK | CARIRGSSCLLTGAFGIW | QSVSSNY | GAS | CQQFGGSPG | 1 |
| MS9_p16w115 | GGSISSSSYY | IYYGGTT | CARDISSTYNWLDPW | QDISSW | AAS | CQQANSFPRTF | 1 |
| MS9_p16w127 | GGSISSHY | FYSSGNT | CARHLGVMYAFNIW | QDIGNY | DAS | CQQYDKYPPTF | 1 |
| MS9_p16w133 | GGSISSYY | IYTSGTT | CARDHGFWSGYYFDYW | QSVLYSSNNKNY | WAS | CQQYYSSPLTF | 1 |
| MS9_p16w15 | GYAL | | CARVRISLFRGAKW*GLSTAT | QSVLYSSNSKNY | WAS | CQQYYTTPPTF | 1 |
| | | | VWTS | | | | |
| MS9_p16w159 | GLTVSRNY | IDSAGST | CARGMTVPGTGYYYYYYMDVW | QSLLHKNGYNY | LGS | CMQGLQKFTF | 1 |
| MS9_p16w162 | GFSVTTTY | INRLGST | CARDQTNTWPGDAFDVW | QGITNS | AAS | CQQYYLLPRTF | 1 |
| MS9_p16w168 | GGSISTSSYY | IYYTGST | CARDLTTGTENGFDIW | ESVSSY | DAF | CQQRISWPRTF | 1 |
| MS9_p16w172 | GFTFSSYG | ISYDGSNK | CAKDRQNYGDYGTSPDYW | QDISNY | DAS | CQQYDNLPLTF | 1 |
| MS9_p16w18 | GYSFTSYW | IDPAGSDT | CARHGRYYDSGTYTYW | TGAVTSGHY | DTS | CLLSYSGARPVF | 1 |
| MS9_p16w2 | GASISSYH | IYSNGNT | CATEARCPGDCYAGSFDYW | QSVGSN | GAS | CQQYNNWPQTF | 1 |
| MS9_p16w24 | EFSLSTSGVG | IYWNDDE | CARTTERCTGGRCYNFNDVFD | QSLLYRSNNKNY | WAS | CQQYYTTVTF | 1 |
| | | | IW | | | | |
| MS9_p16w25 | GYSFTGYW | IDPSDSYT | CATRWRGNLRSGDYYYMDVW | SSNIGSNT | VNN | CAAWDDSLNGVVF | 1 |
| MS9_p16w47 | GGSISSYS | IYHSGST | CARQSSMYAGGPEWFDPW | QSVSSF | DAS | CQQRSSWLITF | 1 |
| MS9_p16w48 | GFSLSNPRMA | IFSNDEK | CARIRVGYNYSPDAFDFW | RGISNY | AAY | CQQLNSYPLTF | 1 |
| MS9_p16w59 | GDSVSSNRAA | TYYNSKWYS | CARGSGYWDFDYW | SSDVGGYNY | DVT | CCSYAGGYTFGVF | 1 |
| MS9_p16w6 | STAA | VGPSGTST | CAKEGDFWSGYYSGYFDLW | QSISSQ | DAS | CQQRFNWPLFTF | 1 |
| MS9_p16w8 | GFTFSSYW | INQDGIEK | CARDGAPRWDYYFYMDVW | QSVTSNF | GAS | CHQYGSTPRTF | 1 |
| MS9_p16w83 | GGSISSTFYY | IYYSGNT | CGRDPWATYSDYVTGWFDPW | QSVSSNY | GAS | CQQYGSSPRTF | 1 |
| MS9_p16w93 | | IWYDGGNK | CASQALGERSVVRGVIGRLVM | QSVSSN | GAS | CQQYSNWPPRITF | 1 |
| | | | DVW | | | | |
| MS9_p16w94 | GGSISSSSYY | IHYSGST | CVRPYCSRTSCYGGHAFDIW | QDISNY | DAS | CQQCDNLPHTF | 1 |

1. A method for determining the presence of EBV-driven pathogenic B cells in an individual, the method comprising:
detecting in a biological sample EBV-infected B cells, and if EBV-infected B cells are detected treating said patient with an EBV-infected B cell depleting or inhibiting therapeutic.

2. The method of claim 1, wherein the individual suffers from an autoimmune disease, optionally multiple sclerosis, or a multiple sclerosis spectrum disorder; systemic lupus erythematosus; type I diabetes; rheumatoid arthritis; Sjogren's syndrome; or dermatomyositis.

3-4. (canceled)

5. The method of claim 1, wherein glialcam epitope is cross-reactive with an EBNA-1 epitope present in the EBV infected B cells.

6. The method of claim 5, wherein the epitope comprises one or both of EBNA-1 residues 386-405, and hepacam/glialcam residues 337-385.

7. The method of claim 1, wherein EBV-driven pathogenic B cells are detected by determining the presence of markers associated with active EBV infection in the B cells, wherein the markers are protein markers or mRNA markers.

8-9. (canceled)

10. The method of claim 7, wherein the markers comprise one or more of BILF-1, LMP1 and LMP2.

11. The method of claim 7, wherein detection of the markers associated with active EBV infection is used to monitor treatment response and guide additional treatment.

12. (canceled)

13. The method of claim 1, wherein the treatment comprises depletion of pathogenic B cells.

14. The method of claim 13, wherein the treatment comprises use of monoclonal antibodies targeting EBV LMP1, LMP2, BILF1 or a combination of these EBV proteins.

15. The method of claim 13, wherein the individual is treated with a depletion agent targeted to markers present on B cells actively infected with EBV.

16. The method of claim 15, wherein the targeted markers comprise EBV proteins: BILF-1, LMP1 and/or LMP2.

17. The method of claim 15, wherein the depletion agent is an antibody.

18. The method of claim 17, wherein the antibody is complexed with a cytotoxic agent.

19. The method of claim 13, wherein the treatment comprises inhibition of pathogenic B cells.

20. The method of claim 19, wherein the B cells are treated with a BTK inhibitor.

21. The method of claim 13, wherein the treatment comprises administration of an agent to tolerize immune cells to a cross-reactive EBNA-1/glialcam epitope.

22. The method of claim 21, wherein the agent is an altered peptide ligand.

23. The method of claim 21, wherein the agent is a DNA construct encoding the cross-reactive epitope.

24. The method of claim 22, wherein administration is oral, nasal, intradermal, transdermal or intramuscular.

25. An antibody specific for a glialcam epitope cross-reactive with an EBNA-1 epitope comprising a set of CDR sequences from the sequences provided in Table 3.
